(19) United States
(12) Patent Application Publication
(10) Pub. No.: US 2017/0275371 A1
(43) Pub. Date: Sep. 28, 2017

(54) Methods Of Treatment

(71) Applicant: GlaxoSmithKline Intellectual Property Development Limited, Brentford (GB)

(72) Inventors: Christopher John FRANCIS, Watertown, MA (US); Hua-Xin GAO, Collegeville, PA (US); Yufeng LI, Collegeville, PA (US); Niranjan YANAMANDRA, Collegeville, PA (US)

(21) Appl. No.: 15/615,032

(22) Filed: Jun. 6, 2017

Related U.S. Application Data
(63) Continuation of application No. PCT/IB2016/053285, filed on Jun. 3, 2016.
(60) Provisional application No. 62/322,906, filed on Apr. 15, 2016.
(60) Provisional application No. 62/300,400, filed on Feb. 26, 2016.
(60) Provisional application No. 62/247,488, filed on Oct. 28, 2015.
(60) Provisional application No. 62/201,828, filed on Aug. 6, 2015.

(51) Int. Cl. C07K 16/28; A61K 31/7028; C07K 16/30; A61K 39/395

(52) U.S. Cl. CPC C07K 16/2878; A61K 39/39558; A61K 31/7028; C07K 16/3046; C07K 2317/24; C07K 2317/565; C07K 2317/75; C07K 2317/56; A61K 2039/505

(57) ABSTRACT

Disclosed herein are combinations of an OX40 modulator and a TLR4 modulator, pharmaceutical compositions thereof, uses thereof, and methods of treatment comprising administering said combination, including uses in cancer.

This application is a continuation application of PCT/IB2016/053285, filed on Jun. 3, 2016, which claims the benefit of U.S. Provisional 62/322,906, filed on Apr. 15, 2016, U.S. Provisional 62/300,400, filed on Feb. 26, 2016, U.S. Provisional 62/247,488, filed on Oct. 28, 2015, and U.S. Provisional 62/201,828, filed on Aug. 6, 2015, all of which are incorporated herein by reference.

### FIELD OF THE INVENTION

The present invention relates to a method of treating cancer in a mammal and to combinations useful in such treatment. In particular, the present invention relates to combinations of anti-OX40 antigen binding proteins (ABPs) and one or more TLR4 modulators.

### BACKGROUND OF THE INVENTION

Effective treatment of hyperproliferative disorders, including cancer, is a continuing goal in the oncology field. Generally, cancer results from the deregulation of the normal processes that control cell division, differentiation and apoptotic cell death and is characterized by the proliferation of malignant cells which have the potential for unlimited growth, local expansion and systemic metastasis. Deregulation of normal processes includes abnormalities in signal transduction pathways and response to factors that differ from those found in normal cells.

Immunotherapies are one approach to treat hyperproliferative disorders. A major hurdle that scientists and clinicians have encountered in the development of various types of cancer immunotherapies has been to break tolerance to self antigen (cancer) in order to mount a robust anti-tumor response leading to tumor regression. Unlike traditional development of small and large molecule agents that target the tumor, cancer immunotherapies target cells of the immune system that have the potential to generate a memory pool of effector cells to induce more durable effects and minimize recurrences.

OX40 is a co-stimulatory molecule involved in multiple processes of the immune system. Antigen binding proteins and antibodies that bind OX40 receptor and modulate OX40 signaling are known in the art and are disclosed as immunotherapy, for example, for cancer.

Aminoalkyl glucosaminide phosphates (AGPs) are synthetic ligands of Toll-like Receptor 4 (TLR4). AGPs are known to be useful as vaccine adjuvants and for stimulating cytokine production, activating macrophages, promoting innate immune response, and augmenting antibody production in immunized animals.

Though there have been many recent advances in the treatment of cancer, there remains a need for more effective and/or enhanced treatment of an individual suffering the effects of cancer. The combinations and methods herein that relate to combining therapeutic approaches for enhancing anti-tumor immunity address this need.

### SUMMARY OF THE INVENTION

Provided herein are combinations of anti-OX40 antigen binding proteins (ABPs) and one or more TLR4 modulators. Also provided are methods of treating cancer in a human with the compositions of the invention, and uses of the combinations for therapy, such as therapy for cancer. Further provided are methods for modulating the immune response of a subject in need of cancer treatment, such as a human, comprising administering to said subject an effective amount of the combinations, e.g., in one or more pharmaceutical compositions.

In one embodiment, the OX40 antigen binding protein is one disclosed in WO2012/027328 (PCT/US2011/048752), international filing date 23 Aug. 2011. In another embodiment, the antigen binding protein comprises the CDRs of an antibody disclosed in WO2012/027328 (PCT/US2011/048752), international filing date 23 Aug. 2011, or CDRs with 90% identity to the disclosed CDR sequences. In a further embodiment the antigen binding protein comprises a VH, a VL, or both of an antibody disclosed in WO2012/027328 (PCT/US2011/048752), international filing date 23 Aug. 2011, or a VH or a VL with 90% identity to the disclosed VH or VL sequences.

In another embodiment, the OX40 antigen binding protein is disclosed in WO2013/028231 (PCT/US2012/024570), international filing date 9 Feb. 2012. In another embodiment, the antigen binding protein comprises the CDRs of an antibody disclosed in WO2013/028231 (PCT/US2012/024570), international filing date 9 Feb. 2012, or CDRs with 90% identity to the disclosed CDR sequences. In a further embodiment, the antigen binding protein comprises a VH, a VL, or both of an antibody disclosed in WO2013/028231 (PCT/US2012/024570), international filing date 9 Feb. 2012, or a VH or a VL with 90% identity to the disclosed VH or VL sequences.

In another embodiment, the anti-OX40 ABP or antibody of the invention comprises one or more of the CDRs or VH or VL sequences, or sequences with 90% identity thereto, shown in the Figures herein.

In one embodiment, the ABP or antibody of the invention comprises the CDRs of the 106-222 antibody, e.g., of FIGS. 6-7 herein, e.g., CDRH1, CDRH2, and CDRH3 having the amino acid sequence as set forth in SEQ ID NOs 1, 2, and 3, as disclosed in FIG. 6, and e.g., CDRL1, CDRL2, and CDRL3 having the sequences as set forth in SEQ ID NOs 7, 8, and 9 respectively. In one embodiment, the ABP or antibody of the invention comprises the CDRs of the 106-222, Hu106 or Hu106-222 antibody as disclosed in WO2012/027328 (PCT/US2011/048752), international filing date 23 Aug. 2011. In a further embodiment, the anti-OX40 ABP or antibody of the invention comprises the VH and VL regions of the 106-222 antibody as shown in FIGS. 6-7 herein, e.g., a VH having an amino acid sequence as set forth in SEQ ID NO:4 and a VL as in FIG. 7 having an amino acid sequence as set forth in SEQ ID NO: 10. In another embodiment, the ABP or antibody of the invention comprises a VH having an amino acid sequence as set forth in SEQ ID NO: 5 in FIG. 6 herein, and a VL having an amino acid sequence as set forth in SEQ ID NO: 11 in FIG. 7 herein. In a further embodiment, the anti-OX40 ABP or antibody of the invention comprises the VH and VL regions of the Hu106-222 antibody or the 106-222 antibody or the Hu106 antibody as disclosed in WO2012/027328 (PCT/US2011/048752), international filing date 23 Aug. 2011. In a further embodiment, the anti-OX40 ABP or antibody of the invention is 106-222, Hu106-222 or Hu106, e.g., as disclosed in WO2012/027328 (PCT/US2011/048752), international filing date 23 Aug. 2011. In a further embodiment, the ABP or antibody of the invention comprises CDRs or VH or VL or antibody sequences with 90% identity to the sequences in this paragraph.

In another embodiment, the anti-OX40 ABP or antibody of the invention comprises the CDRs of the 119-122 antibody, e.g., of FIGS. 10-11 herein, e.g., CDRH1, CDRH2, and CDRH3 having the amino acid sequence as set forth in SEQ ID NOs 13, 14, and 15 respectively. In another embodiment, the anti-OX40 ABP or antibody of the invention comprises the CDRs of the 119-122 or Hull 19 or Hull 19-222 antibody as disclosed in WO2012/027328 (PCT/US2011/048752), international filing date 23 Aug. 2011. In a further embodiment, the anti-OX40 ABP or antibody of the invention comprises a VH having an amino acid sequence as set forth in SEQ ID NO: 16 in FIG. 10 herein, and a VL having the amino acid sequence as set forth in SEQ ID NO: 22 as shown in FIG. 11 herein. In another embodiment, the anti-OX40 ABP or antibody of the invention comprises a VH having an amino acid sequence as set forth in SEQ ID NO: 17 and a VL having the amino acid sequence as set forth in SEQ ID NO: 23. In a further embodiment, the anti-OX40 ABP or antibody of the invention comprises the VH and VL regions of the 119-122 or Hu119 or Hu119-222 antibody as disclosed in WO2012/027328 (PCT/US2011/048752), international filing date 23 Aug. 2011. In a further embodiment, the ABP or antibody of the invention is 119-222 or Hu119 or Hu119-222 antibody, e.g., as disclosed in WO2012/027328 (PCT/US2011/048752), international filing date 23 Aug. 2011. In a further embodiment, the ABP or antibody of the invention comprises CDRs or VH or VL or antibody sequences with 90% identity to the sequences in this paragraph.

In another embodiment, the anti-OX40 ABP or antibody of the invention comprises the CDRs of the 119-43-1 antibody, e.g., as shown in FIGS. 14-15 herein. In another embodiment, the anti-OX40 ABP or antibody of the invention comprises the CDRs of the 119-43-1 antibody as disclosed in WO2013/028231 (PCT/US2012/024570), international filing date 9 Feb. 2012. In a further embodiment, the anti-OX40 ABP or antibody of the invention comprises one of the VH and one of the VL regions of the 119-43-1 antibody as shown in FIGS. 14-17. In a further embodiment, the anti-OX40 ABP or antibody of the invention comprises the VH and VL regions of the 119-43-1 antibody as disclosed in WO2013/028231 (PCT/US2012/024570), international filing date 9 Feb. 2012. In a further embodiment, the ABP or antibody of the invention is 119-43-1 or 119-43-1 chimeric as disclosed in FIGS. 14-17 herein. In a further embodiment, the ABP or antibody of the invention as disclosed in WO2013/028231 (PCT/US2012/024570), international filing date 9 Feb. 2012. In further embodiments, any one of the ABPs or antibodies described in this paragraph are humanized. In further embodiments, any one of the any one of the ABPs or antibodies described in this paragraph are engineered to make a humanized antibody. In a further embodiment, the ABP or antibody of the invention comprises CDRs or VH or VL or antibody sequences with 90% identity to the sequences in this paragraph.

In another embodiment, any mouse or chimeric sequences of any anti-OX40 ABP or antibody of the invention are engineered to make a humanized antibody.

In one embodiment, the anti-OX40 ABP or antibody of the invention comprises: (a) a heavy chain variable region CDR1 comprising the amino acid sequence of SEQ ID NO: 1; (b) a heavy chain variable region CDR2 comprising the amino acid sequence of SEQ ID NO: 2; (c) a heavy chain variable region CDR3 comprising the amino acid sequence of SEQ ID NO. 3; (d) a light chain variable region CDR1 comprising the amino acid sequence of SEQ ID NO. 7; (e) a light chain variable region CDR2 comprising the amino acid sequence of SEQ ID NO. 8; and (f) a light chain variable region CDR3 comprising the amino acid sequence of SEQ ID NO. 9.

In another embodiment, the anti-OX40 ABP or antibody of the invention comprises: (a) a heavy chain variable region CDR1 comprising the amino acid sequence of SEQ ID NO: 13; (b) a heavy chain variable region CDR2 comprising the amino acid sequence of SEQ ID NO: 14; (c) a heavy chain variable region CDR3 comprising the amino acid sequence of SEQ ID NO. 15; (d) a light chain variable region CDR1 comprising the amino acid sequence of SEQ ID NO. 19; (e) a light chain variable region CDR2 comprising the amino acid sequence of SEQ ID NO. 20; and (f) a light chain variable region CDR3 comprising the amino acid sequence of SEQ ID NO. 21.

In another embodiment, the anti-OX40 ABP or antibody of the invention comprises: a heavy chain variable region CDR1 comprising the amino acid sequence of SEQ ID NO: 1 or 13; a heavy chain variable region CDR2 comprising the amino acid sequence of SEQ ID NO: 2 or 14; and/or a heavy chain variable region CDR3 comprising the amino acid sequence of SEQ ID NO: 3 or 15, or a heavy chain variable region CDR having 90% identity thereto.

In yet another embodiment, the anti-OX40 ABP or antibody of the invention comprises: a light chain variable region CDR1 comprising the amino acid sequence of SEQ ID NO: 7 or 19; a light chain variable region CDR2 comprising the amino acid sequence of SEQ ID NO: 8 or 20 and/or a light chain variable region CDR3 comprising the amino acid sequence of SEQ ID NO: 9 or 21, or a heavy chain variable region having 90 percent identity thereto.

In a further embodiment, the anti-OX40 ABP or antibody of the invention comprises: a light chain variable region (“VL”) comprising the amino acid sequence of SEQ ID NO: 10, 11, 22 or 23, or an amino acid sequence with at least 90 percent identity to the amino acid sequences of SEQ ID NO: 10, 11, 22 or 23. In another embodiment, the anti-OX40 ABP or antibody of the invention comprises a heavy chain variable region (“VH”) comprising the amino acid sequence of SEQ ID NO: 4, 5, 16 and 17, or an amino acid sequence with at least 90 percent identity to the amino acid sequences of SEQ ID NO: 4, 5, 16 and 17. In another embodiment, the anti-OX40 ABP or antibody of the invention comprises a variable heavy chain sequence of SEQ ID NO:5 and a variable light chain sequence of SEQ ID NO: 11, or a sequence having 90 percent identity thereto. In another embodiment, the anti-OX40 ABP or antibody of the invention comprises a variable heavy chain sequence of SEQ ID NO: 17 and a variable light chain sequence of SEQ ID NO: 23 or a sequence having 90 percent identity thereto.

In another embodiment, the anti-OX40 ABP or antibody of the invention comprises a variable light chain encoded by the nucleic acid sequence of SEQ ID NO: 12, or 24, or a nucleic acid sequence with at least 90 percent identity to the nucleotide sequences of SEQ ID NO: 12 or 24. In another embodiment, the anti-OX40 ABP or antibody of the invention comprises a variable heavy chain encoded by a nucleic acid sequence of SEQ ID NO: 6 or 18, or a nucleic acid sequence with at least 90 percent identity to nucleotide sequences of SEQ ID NO: 6 or 18.

Also provided herein are monoclonal antibodies. In one embodiment, the monoclonal antibodies comprise a variable light chain comprising the amino acid sequence of SEQ ID NO: 10 or 22, or an amino acid sequence with at least 90 percent identity to the amino acid sequences of SEQ ID NO: 10 or 22. Further provided are monoclonal antibodies comprising a variable heavy chain comprising the amino acid sequence of SEQ ID NO: 4 or 16, or an amino acid sequence with at least 90 percent identity to the amino acid sequences of SEQ ID NO: 4 or 16.

Another embodiment of the invention includes CDRs, VH regions, and VL regions, and antibodies and nucleic acids encoding the same as disclosed in the below Sequence Listing.

### BRIEF DESCRIPTION OF THE DRAWINGS

FIG. 1A is a graph showing dose-dependent anti-tumor activity (as measured by tumor growth inhibition over time) of TLR4 agonist (CRX-527) in a CT-26 syngeneic mouse model of colon cancer. Results are the mean of 10 animals.

FIG. 1B is a graph showing dose-dependent anti-tumor activity (as measured by tumor growth inhibition over time) of a rat anti-mouse OX40 receptor antibody (clone OX-86) in a CT-26 syngeneic mouse model of colon cancer. Results are the mean of 10 animals; control treatments in FIG. 1A are the same as those in FIG. 1B.

FIG. 2 is a graph showing anti-tumor activity (as measured by tumor growth inhibition over time) of a rat anti-mouse OX40 receptor antibody (clone OX-86), 5 ug of TLR4 agonist (CRX-527), and the combination of both in a CT-26 syngeneic mouse model of colon cancer. Results are the mean of 10 animals.

FIG. 3 is a graph showing dose-dependent anti-tumor activity (as measured by tumor growth inhibition over time) of a rat anti-mouse OX40 receptor antibody (clone OX-86), 25 ug of TLR4 agonist (CRX-527), and the combination of both in a CT-26 syngeneic mouse model of colon cancer measured over 38 days. Results are the mean of 10 animals; control treatments in FIG. 2 represent identical animals as those in FIG. 3.

FIGS. 4A-4F are graphs showing dose-dependent anti-tumor activity (as measured by tumor growth inhibition over time) in individual mice of a control antibody (IgG), rat anti-mouse OX40 receptor antibody (clone OX-86), 5 or 25 ug of TLR4 agonist (CRX-527), and the combination of OX86 and CRX-527 in a group of mice in a CT-26 syngeneic mouse model of colon cancer measured over 42 days. The average group tumor volume for mice remaining on study in FIGS. 4A-4F were used to generate the plots in FIGS. 2-3.

FIG. 5 is a graph showing dose-dependent anti-tumor activity (as measured by tumor growth inhibition over time) of 4, 20, or 100 ug of TLR4 agonist (CRX-601) in a CT-26 syngeneic mouse model of colon cancer.

FIGS. 6-12 show sequences of the ABPs and antibodies of the invention, e.g., CDRs and VH and VL sequences.

FIGS. 13-17 show sequences of ABPs and antibodies of the invention, e.g., CDRs and VH and VL sequences.

FIG. 18 is a graph showing dose-dependent anti-tumor activity (as measured by tumor growth inhibition over time) of the TLR4 agonist CRX-601 dosed intratumoral in a CT-26 syngeneic mouse tumor model.

FIG. 19 is a graph showing survival curves of mice treated with the TLR4 agonist CRX-601 intratumoral dosed intratumoral in a CT-26 syngeneic mouse tumor model. (*p-values≦0.05).

FIG. 20 is a graph showing dose-dependent anti-tumor activity (as measured by tumor growth inhibition over time) of the TLR4 agonist CRX-601 in a CT-26 syngeneic mouse tumor model. (*p-values≦0.05)

FIG. 21 is a graph showing survival curves of mice treated with the TLR4 agonist CRX-601 dosed intravenous in a CT-26 syngeneic mouse tumor model (*p-values≦0.05).

FIG. 22 is a graph showing anti-tumor activity (as measured by tumor growth inhibition over time) of 25 ug/mouse of a rat anti-mouse OX40 antibody clone OX-86, dosed Anti-tumor activity (as measured by tumor growth inhibition over time) of 25 ug/mouse of a rat anti-mouse OX40 antibody clone OX-86, dosed via intraperitoneal injection twice per week for 6 doses total, 10 ug or 25 ug/mouse of TLR4 agonist CRX-601 dosed intravenous 1×/week for 3 doses total, and the combination of both in a CT-26 syngeneic mouse model. (*p-values≦0.05)

FIG. 23 is a graph showing survival curves of mice treated with 25 ug/mouse of a rat anti-mouse OX40 receptor antibody (clone OX-86), dosed via intraperitoneal injection twice per week for 6 doses total, 10 ug or 25 ug of TLR4 agonist CRX-601 dosed intravenous 1×/week for 3 doses total, and the combination of both in a CT-26 syngeneic mouse model. (*p-values≦0.05)

FIG. 24 is a graph showing anti-tumor activity (as measured by tumor growth inhibition over time) of 25 ug/mouse of a rat anti-mouse OX40 receptor antibody (clone OX-86), dosed via intraperitoneal injection twice per week for 6 doses total, or 25 ug/mouse of TLR4 agonist CRX-601 dosed intravenous 1×/week for 3 doses total, and the combination of both in a CT-26 syngeneic mouse model. (*p-values≦0.05)

FIG. 25 shows survival curves of mice treated with 25 ug/mouse of a rat anti-mouse OX40 receptor antibody (clone OX-86), dosed via intraperitoneal injection twice per week for 6 doses total, or 25 ug/mouse of TLR4 agonist CRX-601 dosed intravenous 1×/week for 3 doses total, and the combination of both in a CT-26 syngeneic mouse model. (*p-values≦0.05)

FIGS. 26 A-C are graphs showing increase of leukocytes and immune-activation in mice treated with 10 ug of TLR4 agonist CRX-601, 25 ug of a rat anti-mouse OX40 receptor antibody (clone OX-86), and the combination of both in a CT-26 syngeneic mouse model of colon cancer measured at 8 days post-dosing.

FIGS. 27 A-B are graphs showing increases of immune-activating cytokines TNF alpha (A) and IL-12p70 (B) in mice treated with 10 ug of TLR4 agonist CRX-601, a rat anti-mouse OX40R receptor antibody (clone OX-86), and the combination of both in a CT-26 syngeneic mouse model of colon cancer measured at 1 and 8 days post dosing.

FIG. 28 is a graph showing anti-tumor activity (as measured by tumor growth inhibition over time) of 25 ug/mouse of a rat anti-mouse OX40 receptor antibody (clone OX-86), dosed via intraperitoneal injection twice per week for 6 doses total, or 25 ug/mouse of TLR4 agonist CRX-601 dosed intravenous 1×/week for 3 doses total, and the combination of both in a CT-26 syngeneic mouse model. (0.5% Glycerol/4% Dextrose vehicle used for CRX-601). (*p-values<0.05)

FIG. 29 is a graph showing anti-tumor activity (as measured by tumor growth inhibition over time) of 25 ug/mouse of a rat anti-mouse OX40 receptor antibody (clone OX-86), dosed via intraperitoneal injection twice per week for 6 doses total, or 25 ug/mouse of TLR4 agonist CRX-601 dosed intratumoral 1×/week for 3 doses total, and the combination of both in a CT-26 syngeneic mouse model. (0.5% Glycerol/4% Dextrose vehicle used for CRX-601). (*p-values<0.05)

FIG. 30 is a graph showing survival curves of mice treated with 25 ug/mouse of a rat anti-mouse OX40 antibody (clone OX-86), dosed via intraperitoneal injection twice per week for 6 doses total, or 25 ug/mouse of TLR4 agonist CRX-601 dosed intravenous 1×/week for 3 doses total, and the combination of both in a CT-26 syngeneic mouse model. (0.5% Glycerol/4% Dextrose vehicle used for CRX-601) (*p-values<0.05)

FIG. 31 is a graph showing survival curves of mice treated with 25 ug/mouse of a rat anti-mouse OX40 receptor antibody (clone OX-86), dosed via intraperitoneal injection twice per week for 6 doses total, or 25 ug/mouse of TLR4 agonist CRX-601 dosed intratumoral 1×/week for 3 doses total, and the combination of both in a CT-26 syngeneic mouse model. (0.5% Glycerol/4% Dextrose vehicle used for CRX-601) (*p-values<0.05)

FIG. 32 is a graph showing CT-26 tumor re-challenge of tumor-free mice in study 6. 68 days post first dose, tumor-free mice were re-challenged with CT-26 tumor cells. Naïve control mice were also included. While tumors grew as expected in the control naïve mice, tumors were rejected and no tumors grew in the treatment groups.

FIG. 33 is a graph showing anti-tumor activity (as measured by tumor growth inhibition over time) of 25 ug/mouse of a rat anti-mouse OX40 receptor antibody (clone OX-86), dosed via intraperitoneal injection twice per week for 6 doses total, or 25 ug/mouse of TLR4 agonist CRX-601 dosed intravenous 1×/week for 3 doses total, and the combination of both in a CT-26 syngeneic mouse model. (0.5% Glycerol/4% Dextrose vehicle used for CRX-601 intravenous dosing.) (*p-values<0.05)

FIG. 34 is a graph showing anti-tumor activity (as measured by tumor growth inhibition over time) of 25 ug/mouse of a rat anti-mouse OX40 receptor antibody (clone OX-86), dosed via intraperitoneal injection twice per week for 6 doses total, or 25 ug/mouse of TLR4 agonist CRX-601 dosed intratumoral 1×/week for 3 doses total, and the combination of both in a CT-26 syngeneic mouse model. (DOPC/CHOL Liposome formulation used for CRX-601 intratumoral dosing) (*p-values<0.05).

FIG. 35 is a graph showing survival curves of mice treated with 25 ug/mouse of a rat anti-mouse OX40 receptor antibody (clone OX-86), dosed via intraperitoneal injection twice per week for 6 doses total, or 25 ug/mouse of TLR4 agonist CRX-601 dosed intravenous 1×/week for 3 doses total, and the combination of both in a CT-26 syngeneic mouse model. (0.5% Glycerol/4% Dextrose vehicle used for CRX-601 intravenous dosing) (*p-values<0.05).

FIG. 36 is a graph showing survival curves of mice treated with 25 ug/mouse of a rat anti-mouse OX40 receptor antibody (clone OX-86), dosed via intraperitoneal injection twice per week for 6 doses total, or 25 ug/mouse of TLR4 agonist CRX-601 dosed intravenous 1×/week for 3 doses total, and the combination of both in a CT-26 syngeneic mouse model. (0.5% Glycerol/4% Dextrose vehicle used for CRX-601 intravenous dosing) (*p-values<0.05).

FIG. 37 is a graph showing CT-26 tumor re-challenge of tumor-free mice in study 7. 80 days post-first dose, tumor-free mice were re-challenged with CT-26 tumor cells in the number of mice noted. Naïve control mice were also included. While tumors grew as expected in the control naïve mice, tumors were rejected and no tumors grew in the treatment groups.

FIG. 38 is a graph showing tumor growth of individual mice of Group 7: CRX-601 25 ug/mouse (in 0.5% glycerol/4% dextrose) dosed intravenous once per week for 3 doses total+OX86 25 ug/mouse dosed intraperontoneal twice per week for 6 doses total.

FIG. 39 is a graph showing tumor growth of individual mice of Group 8: CRX-601 25 ug/mouse (in 0.5% glycerol/4% dextrose) dosed intratumoral once per week in the left flank tumor for 3 doses total+OX86 25 ug/mouse dosed intraperontoneal twice per week for 6 doses total.

FIG. 40 is a graph showing tumor growth of individual mice of Group 12: CRX-601 25 ug/mouse (in DOPC/CHOL Liposome) dosed intratumoral once per week in the left flank tumor for 3 doses total+OX86 25 ug/mouse dosed intraperontoneal twice per week for 6 doses total

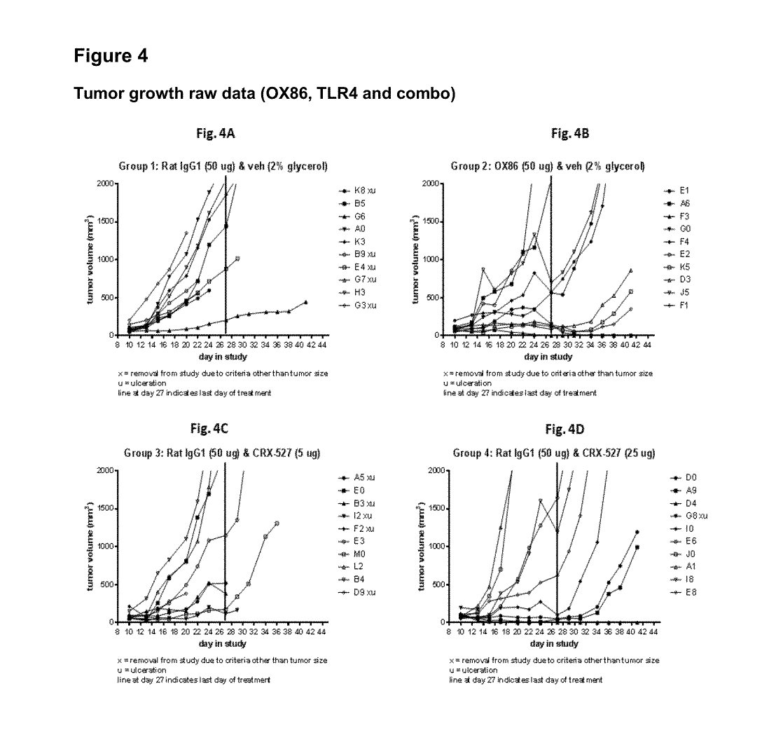

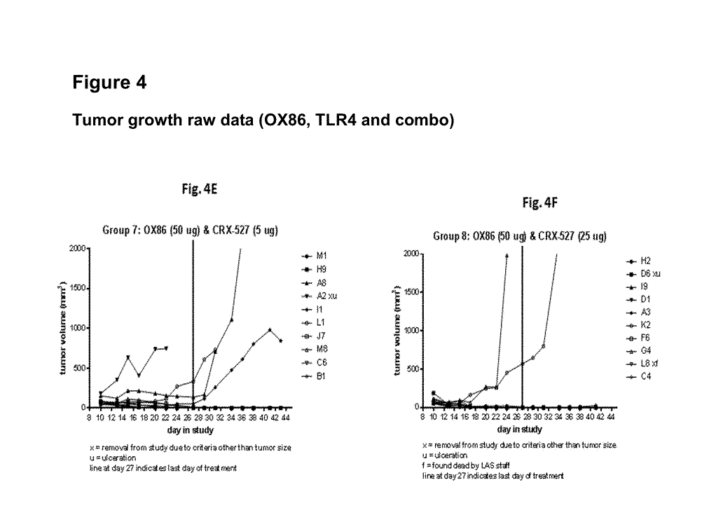

FIG. 4A-4D are graphs showing survival curves for all treatment groups in Study 8. Mice remaining on study by day 60 were completely tumor-free.

FIG. 42A-C are graphs showing upregulation of OX40 expression induced by CRX601 treatment with a range of concentrations (0.01-1000 ng/ml) on human CD4+ T cells (A), dendritic cells (B), and monocytes (C) at 24 hours in in vitro cell culture.

### DETAILED DESCRIPTION OF THE INVENTION

### Compositions and Combinations

Improved function of the immune system is a goal of immunotherapy for cancer. While not being bound by theory, it is thought that for the immune system to be activated and effectively cause regression or eliminate tumors, there must be efficient cross-talk among the various compartments of the immune system as well as at the tumor bed. The tumoricidal effect is dependent on one or more steps, e.g., the uptake of antigen by immature dendritic cells and presentation of processed antigen via MHC I and II by mature dendritic cells to naïve CD8 (cytotoxic) and CD4 (helper) lymphocytes, respectively, in the draining lymph nodes. Naive T cells express molecules, such as CTLA-4 and CD28, that engage with co-stimulatory molecules of the B7 family on antigen presenting cells (APCs) such as dendritic cells. In order to keep T cells in check during immune surveillance, B7 on APCs preferentially binds to CTLA-4, an inhibitory molecule on T lymphocytes. However, upon engagement of the T cell receptor (TCR) with MHC Class I or II receptors via cognate peptide presentation on APCs, the co-stimulatory molecule disengages from CTLA-4 and instead binds to the lower affinity stimulatory molecule CD28, causing T cell activation and proliferation. This expanded population of primed T lymphocytes retains memory of the antigen that was presented to them as they traffic to distant tumor sites.

Upon encountering a tumor cell bearing the cognate antigen, they eliminate the tumor via cytolytic mediators such as granzyme B and perforins. This apparently simplistic sequence of events is highly dependent on several cytokines, co-stimulatory molecules and check point modulators to activate and differentiate these primed T lymphocytes to a memory pool of cells that can eliminate the tumor.

Thus, an emerging immunotherapeutic strategy is to target T cell co-stimulatory molecules, e.g., OX40. OX40 (e.g., hOX40 or hOX40R) is a tumor necrosis factor receptor family member that is expressed, among other cells, on activated CD4 and CD8 T cells. One of its functions is in the differentiation and long-term survival of these cells. The ligand for OX40 (OX40L) is expressed by activated antigen-presenting cells. In one embodiment, the ABPs and antibodies of the invention modulate OX40 and promote growth and/or differentiation of T cells and increase long-term memory T-cell populations, e.g., in overlapping mechanisms as those of OX40L, by “engaging” OX40. Thus, in another embodiment, the ABPs and antibodies of the invention bind and engage OX40. In yet another embodiment, the ABPs and antibodies of the invention modulate OX40. In a further embodiment, the ABPs and antibodies of the invention modulate OX40 by mimicking OX40L. In another embodiment, the ABPs and antibodies of the invention are agonist antibodies. In another embodiment, the ABPs and antibodies of the invention modulate OX40 and cause proliferation of T cells. In a further embodiment, the ABPs and antibodies of the invention modulate OX40 and improve, augment, enhance, or increase proliferation of CD4 T cells. In another embodiment, the ABPs and antibodies of the invention improve, augment, enhance, or increase proliferation of CD8 T cells. In a further embodiment, the ABPs and antibodies of the invention improve, augment, enhance, or increase proliferation of both CD4 and CD8 T cells. In another embodiment, the ABPs and antibodies of the invention enhance T cell function, e.g., of CD4 or CD8 T cells, or both CD4 and CD8 T cells. In a further embodiment, the ABPs and antibodies of the invention enhance effector T cell function. In another embodiment, the ABPs and antibodies of the invention improve, augment, enhance, or increase long-term survival of CD8 T cells. In further embodiments, any of the preceding effects occur in a tumor microenvironment.

Of equal importance is the blockade of a potentially robust immunosuppressive response at the tumor site by mediators produced both by T regulatory cells (Tregs) as well as the tumor itself (e.g., Transforming Growth Factor (TGF-β) and interleukin-10 (IL-10)). An important immune pathogenesis of cancer can be the involvement of Tregs that are found in tumor beds and sites of inflammation. In general, Treg cells occur naturally in circulation and help the immune system to return to a quiet, although vigilant state, after encountering and eliminating external pathogens. Treg cells help to maintain tolerance to self antigens and are naturally suppressive in function, and they phenotypically characterized as CD4+, CD25+, FOXP3+ cells. In order to break tolerance to effectively treat certain cancers, one mode of therapy is to eliminate Tregs preferentially at tumor sites.

Targeting and eliminating Tregs leading to an anti-tumor response has been more successful in tumors that are immunogenic compared to those that are poorly immunogenic. Many tumors secrete cytokines, e.g., TGF-β that may hamper the immune response by causing precursor CD4+25+ cells to acquire the FOXP3+ phenotype and function as Tregs.

“Modulate” as used herein, for example, with regard to a receptor or other target means to change any natural or existing function of the receptor, for example it means affecting binding of natural or artificial ligands to the receptor or target; it includes initiating any partial or full conformational changes or signaling through the receptor or target, and also includes preventing partial or full binding of the receptor or target with its natural or artificial ligands. Also included in the case of membrane bound receptors or targets are any changes in the way the receptor or target interacts with other proteins or molecules in the membrane or change in any localization (or co-localization with other molecules) within membrane compartments as compared to its natural or unchanged state. Modulators are, therefore, compounds or ligands or molecules that modulate a target or receptor.

“Modulate” includes agonizing, e.g., signaling, as well as antagonizing, or blocking signaling or interactions with a ligand or compound or molecule that happen in the unchanged or unmodulated state. Thus, modulators may be agonists or antagonists.

Further, one of skill in the art will recognize that not all modulators will have absolute selectivity for one target or receptor, but are still considered a modulator for that target or receptor; for example, a TLR4 modulator may also engage another TLR, but still be considered a TLR4 modulator. Other modulators are known to have multiple specificities, such as TLR7/8 modulators that modulate both TLR7 and TLR8. Molecules with such known double or multiple specificities are considered a modulator of each of its target; that is, a TLR7/8 modulator is a TLR7 modulator as used herein and likewise a TLR7/8 modulator is a TLR8 modulator as used herein.

“Agonists” of a target or receptor are molecules or compounds or ligands that mimic one or more functions of a natural ligand or molecule that interacts with the target or receptor and includes initiating one or more signaling events through the receptor, mimicking one or more functions of a natural ligand, initiating one or more partial or full conformational changes that are seen in known functioning or signaling through the receptor.

Thus, in one embodiment, the OX40 ABP or antibody inhibits the suppressive effect of Treg cells on other T cells, e.g., within the tumor environment.

Accumulating evidence suggests that the ratio of Tregs to T effector cells in the tumor correlates with anti tumor response. Therefore, in one embodiment, the OX40 ABPs or antibodies of the invention modulate OX40 to augment T effector number and function and inhibit Treg function.

Enhancing, augmenting, improving, increasing, and otherwise changing the anti-tumor effect of OX40 is an object of the invention. Described herein are combinations of an anti-OX40 ABP or antibody of the invention and another compound, such as a TLR modulator described herein.

Thus, as used herein the term “combination of the invention” refers to a combination comprising an anti-OX40 ABP or antibody and a TLR4 modulator, such as an AGP, each of which may be administered separately or simultaneously as described herein.

As used herein, the terms “cancer,” “neoplasm,” and “tumor,” are used interchangeably and in either the singular or plural form, refer to cells that have undergone a malignant transformation or undergone cellular changes that result in aberrant or unregulated growth or hyperproliferation. Such changes or malignant transformations usually make such cells pathological to the host organism, thus precancers or pre-cancerous cells that are or could become pathological and require or could benefit from intervention are also intended to be included. Primary cancer cells (that is, cells obtained from near the site of malignant transformation) can be readily distinguished from non-cancerous cells by well-established techniques, such as histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient. In other words, the terms herein include cells, neoplasms, cancers, and tumors of any stage, including what a clinician refers to as precancer, tumors, in situ growths, as well as late stage metastatic growths, Tumors may be hematopoietic tumor, for example, tumors of blood cells or the like, meaning liquid tumors. Specific examples of clinical conditions based on such a tumor include leukemia such as chronic myelocytic leukemia or acute myelocytic leukemia; myeloma such as multiple myeloma; lymphoma and the like.

As used herein, the term, “agent”, means a substance that produces a desired effect in a tissue, system, animal, mammal, human, or other subject. Accordingly, the term, “anti-neoplastic agent”, means a substance producing an anti-neoplastic effect in a tissue, system, animal, mammal, human, or other subject. The term, “agent”, may be a single compound or a combination or composition of two or more compounds.

By the term “treating” and derivatives thereof as used herein, is meant therapeutic therapy. In reference to a particular condition, treating means: (1) to ameliorate the condition or one or more of the biological manifestations of the condition (2) to interfere with (a) one or more points in the biological cascade that leads to or is responsible for the condition or (b) one or more of the biological manifestations of the condition; (3) to alleviate one or more of the symptoms, effects or side effects associated with the condition or one or more of the symptoms, effects or side effects associated with the condition or treatment thereof; or (4) to slow the progression of the condition or one or more of the biological manifestations of the condition.

As used herein, “prevention” means the prophylactic administration of a drug to substantially diminish the likelihood or severity of a condition or biological manifestation thereof, or to delay the onset of such condition or biological manifestation thereof. The skilled artisan will appreciate that “prevention” is not an absolute term. Prophylactic therapy is appropriate, for example, when a subject is considered at high risk for developing cancer, such as when a subject has a strong family history of cancer or when a subject has been exposed to a carcinogen.

As used herein, the term, “effective amount”, means that amount of a drug or pharmaceutical agent that will elicit the biological or medical response of a tissue, system, animal or human that is being sought, for instance, by a researcher or clinician.

Furthermore, the term, “therapeutically effective amount”, means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount that, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

By the term “combination” and grammatical variations thereof, as used herein, means either simultaneous administration or any manner of separate sequential administration of a therapeutically effective amount of Compound A (an OX-40 ABP) and Compound B (a TLR4 agonist) or a pharmaceutically acceptable salt thereof. Furthermore, it does not matter if the compounds are administered in the same dosage form, e.g., one compound may be administered intravenously and the other compound may be administered intratumorally.

The term “combination kit”, as used herein, means the pharmaceutical composition or compositions that are used to administer Compound A, or a pharmaceutically acceptable salt thereof, and Compound B, or a pharmaceutically acceptable salt thereof, according to the invention. When both compounds are administered simultaneously, the combination kit can contain Compound A, or a pharmaceutically acceptable salt thereof, and Compound B, or a pharmaceutically acceptable salt thereof, in a single pharmaceutical composition, such as a tablet, or in separate pharmaceutical compositions. When the compounds are not administered simultaneously, the combination kit will contain Compound A, or a pharmaceutically acceptable salt thereof, and Compound B, or a pharmaceutically acceptable salt thereof, in separate pharmaceutical compositions. The combination kit can comprise Compound A, or a pharmaceutically acceptable salt thereof, and Compound B, or a pharmaceutically acceptable salt thereof, in separate pharmaceutical compositions in a single package or in separate pharmaceutical compositions in separate packages.

In one embodiment, the invention provides a combination kit comprising the components:
Compound A, or a pharmaceutically acceptable salt thereof, in association with a pharmaceutically acceptable carrier; and
Compound B, or a pharmaceutically acceptable salt thereof, in association with a pharmaceutically acceptable carrier.
In another embodiment, the combination kit comprises the following components:
Compound A, or a pharmaceutically acceptable salt thereof, in association with a pharmaceutically acceptable carrier; and
Compound B, or a pharmaceutically acceptable salt thereof, in association with a pharmaceutically acceptable carrier,
   wherein the components are provided in a form which is suitable for sequential, separate and/or simultaneous administration.

In yet another embodiment, the combination kit comprises:
a first container comprising Compound A, or a pharmaceutically acceptable salt thereof, in association with a pharmaceutically acceptable carrier; and
a second container comprising Compound B, or a pharmaceutically acceptable salt thereof, in association with a pharmaceutically acceptable carrier, and a container means for containing said first and second containers.

The “combination kit” can also be provided by instruction, such as dosage and administration instructions. Such dosage and administration instructions can be of the kind that is provided to a doctor, for example by a drug product label, or they can be of the kind that is provided by a doctor, such as instructions to a patient.

As used herein, the term “Compound A²” means a monoclonal antibody to human OX-40 or the antigen binding portion thereof. Suitably Compound A² means a humanized monoclonal antibody having a heavy chain variable region as set forth in SEQ ID NO: 5 and a light chain variable region as set forth in SEQ ID NO: 11.

As used herein, the term “Compound B²” means a TLR4 agonist of Formula I or Formula Ia. Suitably Compound B² means the TLR4 agonist CRX-601.

Suitably, the combinations of this invention are administered within a “specified period”.

The term “specified period” and grammatical variations thereof, as used herein, means the interval of time between the administration of one of Compound A² and Compound B² and the other of Compound A² and Compound B². Unless otherwise defined, the specified period can include simultaneous administration. Unless otherwise defined, the specified period refers to administration of Compound A² and Compound B² during a single day.

Suitably, if the compounds are administered within a “specified period” and not administered simultaneously, they are both administered within about 24 hours of each other—in this case, the specified period will be about 24 hours; suitably they will both be administered within about 12 hours of each other—in this case, the specified period will be about 12 hours; suitably they will both be administered within about 11 hours of each other—in this case, the specified period will be about 11 hours; suitably they will both be administered within about 10 hours of each other—in this case, the specified period will be about 10 hours; suitably they will both be administered within about 9 hours of each other —in this case, the specified period will be about 9 hours; suitably they will both be administered within about 8 hours of each other—in this case, the specified period will be about 8 hours; suitably they will both be administered within about 7 hours of each other—in this case, the specified period will be about 7 hours; suitably they will both be administered within about 6 hours of each other—in this case, the specified period will be about 6 hours; suitably they will both be administered within about 5 hours of each other—in this case, the specified period will be about 5 hours; suitably they will both be administered within about 4 hours of each other—in this case, the specified period will be about 4 hours; suitably they will both be administered within about 3 hours of each other—in this case, the specified period will be about 3 hours; suitably they will be administered within about 2 hours of each other—in this case, the specified period will be about 2 hours; suitably they will both be administered within about 1 hour of each other—in this case, the specified period will be about 1 hour. As used herein, the administration of Compound A² and Compound B² in less than about 45 minutes apart is considered simultaneous administration.

Suitably, when the combination of the invention is administered for a “specified period”, the compounds will be co-administered for a “duration of time”.

The term “duration of time” and grammatical variations thereof, as used herein means that both compounds of the invention are administered for an indicated number of consecutive days. Unless otherwise defined, the number of consecutive days does not have to commence with the start of treatment or terminate with the end of treatment, it is only required that the number of consecutive days occur at some point during the course of treatment.

Regarding “specified period” administration: suitably, both compounds will be administered within a specified period for at least one day—in this case, the duration of time will be at least one day; suitably, during the course to treatment, both compounds will be administered within a specified period for at least 3 consecutive days—in this case, the duration of time will be at least 3 days; suitably, during the course to treatment, both compounds will be administered within a specified period for at least 5 consecutive days—in this case, the duration of time will be at least 5 days; suitably, during the course to treatment, both compounds will be administered within a specified period for at least 7 consecutive days—in this case, the duration of time will be at least 7 days; suitably, during the course to treatment, both compounds will be administered within a specified period for at least 14 consecutive days—in this case, the duration of time will be at least 14 days; suitably, during the course to treatment, both compounds will be administered within a specified period for at least 30 consecutive days—in this case, the duration of time will be at least 30 days.

Suitably, if the compounds are not administered during a “specified period”, they are administered sequentially. By the term “sequential administration”, and grammatical derivates thereof, as used herein is meant that one of Compound A² and Compound B² is administered once a day for two or more consecutive days and the other of Compound A² and Compound B² is subsequently administered once a day for two or more consecutive days. Also, contemplated herein is a drug holiday utilized between the sequential administration of one of Compound A² and Compound B² and the other of Compound A² and Compound B². As used herein, a drug holiday is a period of days after the sequential administration of one of Compound A² and Compound B² and before the administration of the other of Compound A² and Compound B² where neither Compound A² nor Compound B² is administered. Suitably the drug holiday will be a period of days selected from: 1 day, 2 days, 3 days, 4 days, 5 days, 6 days, 7 days, 8 days, 9 days, 10 days, 11 days, 12 days, 13 days and 14 days.

Regarding sequential administration: suitably, one of Compound A² and Compound B² is administered for from 1 to 30 consecutive days, followed by an optional drug holiday, followed by administration of the other of Compound A² and Compound B² for from 1 to 30 consecutive days. Suitably, one of Compound A² and Compound B² is administered for from 1 to 21 consecutive days, followed by an optional drug holiday, followed by administration of the other of Compound A² and Compound B² for from 1 to 21 consecutive days. Suitably, one of Compound A² and Compound B² is administered for from 1 to 14 consecutive days, followed by a drug holiday of from 1 to 14 days, followed by administration of the other of Compound A² and Compound B² for from 1 to 14 consecutive days. Suitably, one of Compound A² and Compound B² is administered for from 1 to 7 consecutive days, followed by a drug holiday of from 1 to 10 days, followed by administration of the other of Compound A² and Compound B² for from 1 to 7 consecutive days.

Suitably, Compound B² will be administered first in the sequence, followed by an optional drug holiday, followed by administration of Compound A². Suitably, Compound B² is administered for from 3 to 21 consecutive days, followed by an optional drug holiday, followed by administration of Compound A² for from 3 to 21 consecutive days. Suitably, Compound B² is administered for from 3 to 21 consecutive days, followed by a drug holiday of from 1 to 14 days, followed by administration of Compound A² for from 3 to 21 consecutive days. Suitably, Compound B² is administered for from 3 to 21 consecutive days, followed by a drug holiday of from 3 to 14 days, followed by administration of Compound A² for from 3 to 21 consecutive days. Suitably, Compound B² is administered for 21 consecutive days, followed by an optional drug holiday, followed by administration of Compound A² for 14 consecutive days. Suitably, Compound B² is administered for 14 consecutive days, followed by a drug holiday of from 1 to 14 days, followed by administration of Compound A² for 14 consecutive days. Suitably, Compound B² is administered for 7 consecutive days, followed by a drug holiday of from 3 to 10 days, followed by administration of Compound A² for 7 consecutive days. Suitably, Compound B² is administered for 3 consecutive days, followed by a drug holiday of from 3 to 14 days, followed by administration of Compound A² for 7 consecutive days. Suitably, Compound B² is administered for 3 consecutive days, followed by a drug holiday of from 3 to 10 days, followed by administration of Compound A² for 3 consecutive days.

It is understood that a “specified period” administration and a “sequential” administration can be followed by repeat dosing or can be followed by an alternate dosing protocol, and a drug holiday may precede the repeat dosing or alternate dosing protocol.

The methods of the present invention may also be employed with other therapeutic methods of cancer treatment.

While it is possible that, for use in therapy, therapeutically effective amounts of the combinations of the present invention may be administered as the raw chemical, it is preferable to present the combinations as a pharmaceutical composition or compositions. Accordingly, the invention further provides pharmaceutical compositions, which include Compound A² and/or Compound B², and one or more pharmaceutically acceptable carriers. The combinations of the present invention are as described above. The carrier(s) must be acceptable in the sense of being compatible with the other ingredients of the formulation, capable of pharmaceutical formulation, and not deleterious to the recipient thereof. In accordance with another aspect of the invention there is also provided a process for the preparation of a pharmaceutical formulation including admixing Compound A² and/or Compound B² with one or more pharmaceutically acceptable carriers. As indicated above, such elements of the pharmaceutical combination utilized may be presented in separate pharmaceutical compositions or formulated together in one pharmaceutical formulation.

Pharmaceutical formulations may be presented in unit dose forms containing a predetermined amount of active ingredient per unit dose. As is known to those skilled in the art, the amount of active ingredient per dose will depend on the condition being treated, the route of administration and the age, weight and condition of the patient. Preferred unit dosage formulations are those containing a daily dose or sub-dose, or an appropriate fraction thereof, of an active ingredient. Furthermore, such pharmaceutical formulations may be prepared by any of the methods well known in the pharmacy art.

Compound A² and Compound B² may be administered by any appropriate route. Suitable routes include oral, rectal, nasal, topical (including buccal and sublingual), intratumorally, vaginal, and parenteral (including subcutaneous, intramuscular, intravenous, intradermal, intrathecal, and epidural). It will be appreciated that the preferred route may vary with, for example, the condition of the recipient of the combination and the cancer to be treated. It will also be appreciated that each of the agents administered may be administered by the same or different routes and that Compound A² and Compound B² may be compounded together in a pharmaceutical composition/formulation.

The administration of a therapeutically effective amount of the combinations of the invention (or therapeutically effective amounts of each of the components of the combination) are advantageous over the individual component compounds in that the combinations provide one or more of the following improved properties when compared to the individual administration of a therapeutically effective amount of a component compound: i) a greater anti-cancer effect than the most active single agent; ii) synergistic or highly synergistic anti-cancer activity; iii) a dosing protocol that provides enhanced anti-cancer activity with reduced side effect profile; iv) a reduction in the toxic effect profile, v) an increase in the therapeutic window; or vi) an increase in the bioavailability of one or both of the component compounds.

The invention further provides pharmaceutical compositions, which include one or more of the components herein, and one or more pharmaceutically acceptable carriers, diluents, or excipients. The combination of the invention may comprise two pharmaceutical compositions, one comprising an ABP or antibody of the invention, and the other comprising a TLR4 modulator, each of which may have the same or different carriers, diluents or excipients. The carrier(s), diluent(s) or excipient(s) must be acceptable in the sense of being compatible with the other ingredients of the formulation, capable of pharmaceutical formulation, and not deleterious to the recipient thereof. In one embodiment of the invention, the formulation may be aqueous or liposomal. In one embodiment, the liposomal formulation may be a DOPC/CHOL Liposome formulation

The components of the combination of the invention, and pharmaceutical compositions comprising such components may be administered in any order, and in different routes; the components and pharmaceutical compositions comprising the same may be administered simultaneously.

In accordance with another aspect of the invention there is also provided a process for the preparation of a pharmaceutical composition including admixing a component of the combination of the invention and one or more pharmaceutically acceptable carriers, diluents or excipients.

The components of the invention may be administered by any appropriate route. For some components, suitable routes include oral, rectal, nasal, topical (including buccal and sublingual), vaginal, and parenteral (including subcutaneous, intramuscular, intraveneous, intradermal, intrathecal, and epidural). The preferred route may vary with, for example, the condition of the recipient of the combination and the cancer to be treated. Each of the agents administered may be administered by the same or different routes, and the components may be compounded together or in separate pharmaceutical compositions.

In one embodiment, one or more components of a combination of the invention are administered intravenously. In another embodiment, one or more components of a combination of the invention are administered intratumorally. In another embodiment, one or more components of a combination of the invention are administered systemically, e.g., intravenously, and one or more other components of a combination of the invention are administered intratumorally. In another embodiment, all of the components of a combination of the invention are administered systemically, e.g., intravenously. In an alternative embodiment, all of the components of the combination of the invention are administered intratumorally. In any of the embodiments, e.g., in this paragraph, the components of the invention are administered as one or more pharmaceutical compositions.

Antigen Binding Proteins and Antibodies that Bind OX40

“Antigen Binding Protein (ABP)” means a protein that binds an antigen, including antibodies or engineered molecules that function in similar ways to antibodies. Such alternative antibody formats include triabody, tetrabody, miniantibody, and a minibody, Also included are alternative scaffolds in which the one or more CDRs of any molecules in accordance with the disclosure can be arranged onto a suitable non-immunoglobulin protein scaffold or skeleton, such as an affibody, a SpA scaffold, an LDL receptor class A domain, an avimer (see, e.g., U.S. Patent Application Publication Nos. 2005/0053973, 2005/0089932, 2005/0164301) or an EGF domain. An ABP also includes antigen binding fragments of such antibodies or other molecules. Further, an ABP may comprise the VH regions of the invention formatted into a full length antibody, a (Fab′)2 fragment, a Fab fragment, a bi-specific or biparatopic molecule or equivalent thereof (such as scFV, bi- tri- or tetra-bodies, Tandabs, etc.), when paired with an appropriate light chain. The ABP may comprise an antibody that is an IgG1, IgG2, IgG3, or IgG4; or IgM; IgA, IgE or IgD or a modified variant thereof. The constant domain of the antibody heavy chain may be selected accordingly. The light chain constant domain may be a kappa or lambda constant domain. The ABP may also be a chimeric antibody of the type described in WO86/01533, which comprises an antigen binding region and a non-immunoglobulin region.

Thus, herein an ABP of the invention or an anti-OX40 antigen binding protein is one that binds OX40, and in some embodiments, does one or more of the following: modulate signaling through OX40, modulates the function of OX40, agonize OX40 signaling, stimulate OX40 function, or co-stimulate OX40 signaling. Example 1 of U.S. Pat. No. 9,006,399 discloses an OX40 binding assay. One of skill in the art would readily recognize a variety of other well known assays to establish such functions.

The term “antibody” as used herein refers to molecules with an antigen binding domain, and optionally an immunoglobulin-like domain or fragment thereof and includes monoclonal (for example IgG, IgM, IgA, IgD or IgE and modified variants thereof), recombinant, polyclonal, chimeric, humanized, biparatopic, bispecific and heteroconjugate antibodies, or a closed conformation multispecific antibody. An “antibody” included xenogeneic, allogeneic, syngeneic, or other modified forms thereof. An antibody may be isolated or purified. An antibody may also be recombinant, i.e., produced by recombinant means; for example, an antibody that is 90% identical to a reference antibody may be generated by mutagenesis of certain residues using recombinant molecular biology techniques known in the art. Thus, the antibodies of the present invention may comprise heavy chain variable regions and light chain variable regions of the invention which may be formatted into the structure of a natural antibody or formatted into a full length recombinant antibody, a (Fab′)2 fragment, a Fab fragment, a bi-specific or biparatopic molecule or equivalent thereof (such as scFV, bi- tri- or tetra-bodies, Tandabs etc.), when paired with an appropriate light chain. The antibody may be an IgG1, IgG2, IgG3, or IgG4 or a modified variant thereof. The constant domain of the antibody heavy chain may be selected accordingly. The light chain constant domain may be a kappa or lambda constant domain. The antibody may also be a chimeric antibody of the type described in WO86/01533 which comprises an antigen binding region and a non-immunoglobulin region.

One of skill in the art will recognize that the ABPs and antibodies of the invention bind an epitope of OX40. The epitope of an ABP is the region of its antigen to which the ABP binds. Two ABPs bind to the same or overlapping epitope if each competitively inhibits (blocks) binding of the other to the antigen. That is, a 1×, 5×, 10×, 20× or 100× excess of one antibody inhibits binding of the other by at least 50%, 75%, 90% or even 99% as measured in a competitive binding assay compared to a control lacking the competing antibody (see, e.g., Junghans, et al., Cancer Res. 50:1495, 1990. Alternatively, two antibodies have the same epitope if essentially all amino acid mutations in the antigen that reduce or eliminate binding of one antibody reduce or eliminate binding of the other. In addition, the same epitope may include “overlapping epitopes”, e.g., if some amino acid mutations that reduce or eliminate binding of one antibody reduce or eliminate binding of the other.

The strength of binding may be important in dosing and administration of an ABP or antibody of the invention. In one embodiment, the ABP or antibody of the invention binds to OX40, preferably human OX40, with high affinity. For example, when measured by Biacore®, the antibody binds to OX40, preferably human OX40, with an affinity of 1-1000 nM or 500 nM or less or an affinity of 200 nM or less or an affinity of 100 nM or less or an affinity of 50 nM or less or an affinity of 500 pM or less or an affinity of 400 pM or less, or 300 pM or less. In a further aspect the antibody binds to OX40, preferably human OX40, when measured by BIACORE® of between about 50 nM and about 200 nM or between about 50 nM and about 150 nM. In one aspect of the present invention the antibody binds OX40, preferably human OX40, with an affinity of less than 100 nM.

In a further embodiment, binding is measured by BIACORE®. Affinity is the strength of binding of one molecule, e.g., an antibody of the invention, to another, e.g., its target antigen, at a single binding site. The binding affinity of an antibody to its target may be determined by equilibrium methods (e.g., enzyme-linked immunoabsorbent assay (ELISA) or radioimmunoassay (RIA)), or kinetics (e.g., BIACORE® analysis). For example, the BIACORE® methods known in the art may be used to measure binding affinity.

Avidity is the sum total of the strength of binding of two molecules to one another at multiple sites, e.g., taking into account the valency of the interaction.

In an aspect, the equilibrium dissociation constant (KD) of the ABP or antibody of the invention and OX40, preferably human OX40, interaction is 100 nM or less, 10 nM or less, 2 nM or less or 1 nM or less. Alternatively the KD may be between 5 and 10 nM; or between 1 and 2 nM. The KD may be between 1 pM and 500 pM; or between 500 pM and 1 nM. A skilled person will appreciate that the smaller the KD numerical value, the stronger the binding. The reciprocal of KD (i.e., 1/KD) is the equilibrium association constant (KA) having units M-1. A skilled person will appreciate that the larger the KA numerical value, the stronger the binding.

The dissociation rate constant (kd) or “off-rate” describes the stability of the complex of ABP or antibody on one hand and OX40, preferably human OX40 on the other hand, i.e., the fraction of complexes that decay per second. For example, a kd of 0.01 s−1 equates to 1% of the complexes decaying per second. In one embodiment, the dissociation rate constant (kd) is 1×10−3 s−1 or less, 1×10−4 s−1 or less, 1×10−5 s−1 or less, or 1×10−6 s−1 or less. The kd may be between 1×10−5 s−1 and 1×10−4 s−1; or between 1×10−4 s−1 and 1×10−3 s−1.

Competition between an anti-OX40 ABP or antibody of the invention, and a reference antibody, e.g., for binding OX40, an epitope of OX40, or a fragment of the OX40, may be determined by competition ELISA, FMAT or BIAcore®. In one aspect, the competition assay is carried out by BIAcore®. There are several possible reasons for this competition: the two proteins may bind to the same or overlapping epitopes, there may be steric inhibition of binding, or binding of the first protein may induce a conformational change in the antigen that prevents or reduces binding of the second protein.

“Binding fragments” as used herein means a portion or fragment of the ABPs or antibodies of the invention that include the antigen-binding site and are capable of binding OX40 as defined herein, e.g., but not limited to capable of binding to the same epitope of the parent or full length antibody.

Functional fragments of the ABPs and antibodies of the invention are contemplated herein.

Thus, “binding fragments” and “functional fragments” may be Fab and F(ab′)2 fragments that lack the Fc fragment of an intact antibody, clear more rapidly from the circulation, and may have less non-specific tissue binding than an intact antibody (Wahl, et al., J. Nuc. Med. 24:316-325 (1983)). Also included are Fv fragments (Hochman, et al., Biochemistry 12:1130-1135 (1973); Sharon, et al, Biochemistry 15:1591-1594 (1976)). These various fragments are produced using conventional techniques such as protease cleavage or chemical cleavage (see, e.g., Rousseaux, et al., Meth. Enzymol., 121:663-69 (1986)).

“Functional fragments”, as used herein, means a portion or fragment of the ABPs or antibodies of the invention that include the antigen-binding site and are capable of binding the same target as the parent ABP or antibody, e.g., but not limited to, binding the same epitope, and that also retain one or more modulating or other functions described herein or known in the art.

As the ABPs and antibodies of the present invention may comprise heavy chain variable regions and light chain variable regions of the invention which may be formatted into the structure of a natural antibody, a functional fragment is one that retains binding or one or more functions of the full length ABP or antibody as described herein. A binding fragment of an ABP or antibody of the invention may therefore comprise the VL or VH regions, a (Fab′)2 fragment, a Fab fragment, a fragment of a bi-specific or biparatopic molecule or equivalent thereof (such as scFV, bi- tri- or tetra-bodies, Tandabs etc.), when paired with an appropriate light chain.

The term, “CDR”, as used herein, refers to the complementarity determining region amino acid sequences of an antigen binding protein. These are the hypervariable regions of immunoglobulin heavy and light chains. There are three heavy chain and three light chain CDRs (or CDR regions) in the variable portion of an immunoglobulin.

It will be apparent to those skilled in the art that there are various numbering conventions for CDR sequences; Chothia (Chothia et al. (1989) Nature 342: 877-883), Kabat (Kabat et al., Sequences of Proteins of Immunological Interest, 4th Ed., U.S. Department of Health and Human Services, National Institutes of Health (1987)), AbM (University of Bath) and Contact (University College London). The minimum overlapping region using at least two of the Kabat, Chothia, AbM and contact methods can be determined to provide the “minimum binding unit”. The minimum binding unit may be a subportion of a CDR. The structure and protein folding of the antibody may mean that other residues are considered part of the CDR sequence and would be understood to be so by a skilled person. It is noted that some of the CDR definitions may vary depending on the individual publication used.

Unless otherwise stated and/or in absence of a specifically identified sequence, references herein to “CDR”, “CDRL1”, “CDRL2”, “CDRL3”, “CDRH1”, “CDRH2”, “CDRH3” refer to amino acid sequences numbered according to any of the known conventions; alternatively, the CDRs are referred to as “CDR1,” “CDR2,” “CDR3” of the variable light chain and “CDR1,” “CDR2,” and “CDR3” of the variable heavy chain. In some embodiments, the numbering convention is the Kabat convention.

The term, “CDR variant”, as used herein, refers to a CDR that has been modified by at least one, for example 1, 2 or 3, amino acid substitution(s), deletion(s) or addition(s), wherein the modified antigen binding protein comprising the CDR variant substantially retains the biological characteristics of the antigen binding protein pre-modification. It will be appreciated that each CDR that can be modified may be modified alone or in combination with another CDR. In one aspect, the modification is a substitution, particularly a conservative substitution, for example as shown in Table 1.

TABLE 1

| Side chain | Members |
|---|---|
| Hydrophobic | Met, Ala, Val, Leu, Ile |
| Neutral hydrophilic | Cys, Ser, Thr |
| Acidic | Asp, Glu |
| Basic | Asn, Gln, His, Lys, Arg |
| Residues that influence chain orientation | Gly, Pro |
| Aromatic | Trp, Tyr, Phe |

For example, in a variant CDR, the amino acid residues of the minimum binding unit may remain the same, but the flanking residues that comprise the CDR as part of the Kabat or Chothia definition(s) may be substituted with a conservative amino acid residue.

Such antigen binding proteins comprising modified CDRs or minimum binding units as described above may be referred to herein as “functional CDR variants” or “functional binding unit variants”.

The antibody may be of any species, or modified to be suitable to administer to a cross species. For example the CDRs from a mouse antibody may be humanized for administration to humans. In any embodiment, the antigen binding protein is optionally a humanized antibody.

A “humanized antibody” refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity (see, e.g., Queen, et al., Proc. Natl Acad Sci USA, 86:10029-10032 (1989), Hodgson, et al., Bio/Technology, 9:421 (1991)). A suitable human acceptor antibody may be one selected from a conventional database, e.g., the KABAT® database, Los Alamos database, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody. The prior art describes several ways of producing such humanised antibodies—see for example EP-A-0239400 and EP-A-054951.

In yet a further embodiment, the humanized antibody has a human antibody constant region that is an IgG. In another embodiment, the IgG is a sequence as disclosed in any of the above references or patent publications.

For nucleotide and amino acid sequences, the term “identical” or “identity” indicates the degree of identity between two nucleic acid or two amino acid sequences when optimally aligned and compared with appropriate insertions or deletions.

The percent identity between two sequences is a function of the number of identical positions shared by the sequences (i.e., % identity=number of identical positions/total number of positions multiplied by 100), taking into account the number of gaps, and the length of each gap, which need to be introduced for optimal alignment of the two sequences. The comparison of sequences and determination of percent identity between two sequences can be accomplished using a mathematical algorithm, as described below.

Percent identity between a query nucleic acid sequence and a subject nucleic acid sequence is the “Identities” value, expressed as a percentage, which is calculated by the BLASTN algorithm when a subject nucleic acid sequence has 100% query coverage with a query nucleic acid sequence after a pair-wise BLASTN alignment is performed. Such pair-wise BLASTN alignments between a query nucleic acid sequence and a subject nucleic acid sequence are performed by using the default settings of the BLASTN algorithm available on the National Center for Biotechnology Institute's website with the filter for low complexity regions turned off. Importantly, a query nucleic acid sequence may be described by a nucleic acid sequence identified in one or more claims herein.

Percent identity between a query amino acid sequence and a subject amino acid sequence is the “Identities” value, expressed as a percentage, which is calculated by the BLASTP algorithm when a subject amino acid sequence has 100% query coverage with a query amino acid sequence after a pair-wise BLASTP alignment is performed. Such pair-wise BLASTP alignments between a query amino acid sequence and a subject amino acid sequence are performed by using the default settings of the BLASTP algorithm available on the National Center for Biotechnology Institute's website with the filter for low complexity regions turned off. Importantly, a query amino acid sequence may be described by an amino acid sequence identified in one or more claims herein.

In any embodiment of the invention herein, the ABP or antibody may have any one or all CDRs, VH, VL, with 100, 99, 98, 97, 96, 95, 94, 93, 92, 91, or 90 percent identity to the sequence shown or referenced, e.g., as defined by a SEQ ID NO disclosed herein.

ABPs and antibodies that bind human OX40 receptor are provided herein (i.e., an anti-OX40 ABP and an anti-human OX40 receptor (hOX40R) antibody, sometimes referred to herein as an “anti-OX40 ABP or an anti-OX40 antibody” and/or other variations of the same). These antibodies are useful in the treatment or prevention of acute or chronic diseases or conditions whose pathology involves OX40 signaling. In one aspect, an antigen binding protein, or isolated human antibody or functional fragment of such protein or antibody, that binds to human OX40R and is effective as a cancer treatment or treatment against disease is described, for example in combination with another compound such as a TLR4 modulator or TLR4 agonist. Any of the antigen binding proteins or anti-OX40 antibodies disclosed herein may be used as a medicament. Any one or more of the antigen binding proteins or anti-OX40 antibodies may be used in the methods or compositions to treat cancer, e.g., those disclosed herein.

The isolated antibodies as described herein bind to OX40, and may bind to OX40 encoded from the following genes: NCBI Accession Number NP_003317, Genpept Accession Number P23510, or genes having 90 percent homology or 90 percent identity thereto. The isolated antibody provided herein may further bind to the OX40 receptor having one of the following GenBank Accession Numbers: AAB39944, CAE11757, or AAI05071.

Antigen binding proteins and antibodies that bind and/or modulate OX40 receptor are known in the art. Exemplary ABPs and antibodies of the invention are disclosed, for example in International Publication No. WO2013/028231 (PCT/US2012/024570), international filing date 9 Feb. 2012, and WO2012/027328 (PCT/US2011/048752), international filing date 23 Aug. 2011. (To the extent any definitions conflict, this instant application controls). In one embodiment, OX40 antibodies of the present invention are disclosed in U.S. Pat. No. 9,163,085.

### TLR4 Modulators

The combinations of the invention comprise TLR4 “modulators”, that is, molecules that modulate TLR4, for example, by binding and initiating conformational changes or signaling by engaging TLR4, molecules that block binding with a TLR4 ligand.

In one embodiment, TLR4 modulators are aminoalkyl glucosaminide phosphate compounds (AGPs). TLR4 recognizes bacterial LPS (lipopolysaccharide) and when activated initiates an innate immune response. AGPs are a monosaccharide mimetic of the lipid A protein of bacterial LPS and have been developed with ether and ester linkages on the “acyl chains” of the compound. Processes for making these compounds are known and disclosed, for example, in WO 2006/016997, U.S. Pat. Nos. 7,288,640 and 6,113,918, and WO 01/90129. Other AGPs and related processes are disclosed in U.S. Pat. No. 7,129,219, U.S. Pat. No. 6,525,028 and U.S. Pat. No. 6,911,434. AGPs with ether linkages on the acyl chains employed in the composition of the invention are known and disclosed in WO 2006/016997. The AGP compounds set forth and described according to Formula (III) at paragraphs [0019] through [0021] in WO 2006/016997 may be employed in the presently claimed methods and combinations.

AGP compounds employed in the present invention have the structure set forth in Formula 1 as follows:

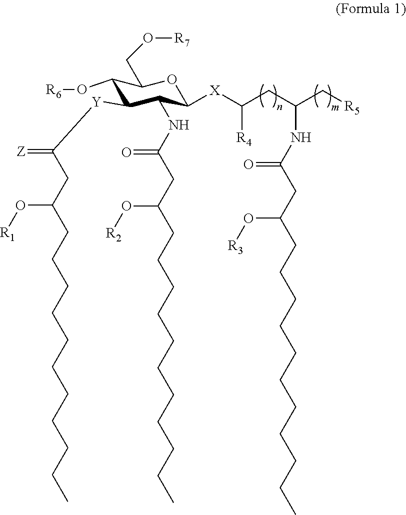

wherein
m is 0 to 6
n is 0 to 4;
X is O or S, preferably O;
Y is O or NH;
Z is O or H;
each R1, R2, R3 is selected independently from the group consisting of a C1-20 acyl and a C1-20 alkyl;
R4 is H or Me;
R5 is selected independently from the group consisting of —H, —OH, —(C1-C4) alkoxy, —PO3R8R9, —OPO3R8R9, —SO3R8, —OSO3R8, —NR8R9, —SR8, —CN, —NO2, —CHO, —CO2R8, and —CONR8R9, wherein R8 and R9 are each independently selected from H and (C1-C4) alkyl; and
each R6 and R7 is independently H or PO3H2.

In Formula 1 the configuration of the 3′ stereogenic centers to which the normal fatty acyl residues (that is, the secondary acyloxy or alkoxy residues, e.g., R10, R20, and R30) are attached is R or S, preferably R (as designated by Cahn-Ingold-Prelog priority rules). Configuration of aglycon stereogenic centers to which R4 and R5 are attached can be R or S. All stereoisomers, both enantiomers and diastereomers, and mixtures thereof, are considered to fall within the scope of the present invention.

The number of carbon atoms between heteroatom X and the aglycon nitrogen atom is determined by the variable “n”, which can be an integer from 0 to 4, or an integer from 0 to 2.

The chain length of normal fatty acids R1, R2, and R3 can be from about 6 to about 16 carbons, or from about 9 to about 14 carbons. The chain lengths can be the same or different. Some embodiments include chain lengths where R1, R2 and R3 are 6 or 10 or 12 or 14.

Formula 1 encompasses L/D-seryl, -threonyl, -cysteinyl ether and ester lipid AGPs, both agonists and antagonists and their homologs (n=1-4), as well as various carboxylic acid bioisosteres (i.e. R5 is an acidic group capable of salt formation; the phosphate can be either on 4- or 6-position of the glucosamine unit, preferably, is in the 4-position).

In a one embodiment of the invention employing an AGP compound of Formula 1, n is 0, R5 is CO2H, R6 is PO3H2, and R7 is H. This AGP compound is set forth as the structure in Formula 1a as follows:

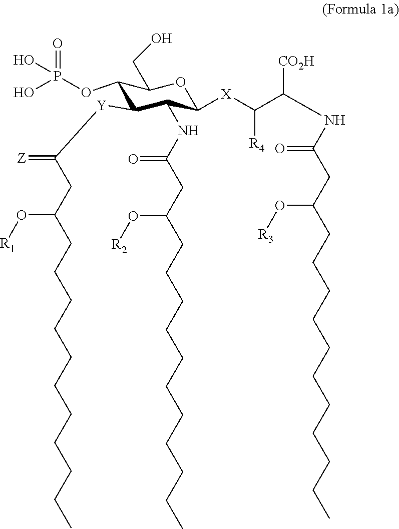

wherein X is O or S; Y is O or NH; Z is O or H; each R1, R2, R3 is selected independently from the group consisting of a C1-20 acyl and a C1-20 alkyl; and R4 is H or methyl.

In Formula 1a the configuration of the 3′ stereogenic centers to which the normal fatty acyl residues (that is, the secondary acyloxy or alkoxy residues, e.g., RO1, R20, and R30) are attached as R or S, preferably R (as designated by Cahn-Ingold-Prelog priority rules).

Configuration of aglycon stereogenic centers to which R4 and CO2H are attached can be R or S. All stereoisomers, both enantiomers and diastereomers, and mixtures thereof, are considered to fall within the scope of the present invention.

Formula 1a encompasses L/D-seryl, -threonyl, -cysteinyl ether or ester lipid AGPs, both agonists and antagonists.

In both Formula 1 and Formula 1a, Z is O attached by a double bond or two hydrogen atoms which are each attached by a single bond. That is, the compound is ester-linked when Z═Y═O; amide-linked when Z═O and Y═NH; and ether-linked when Z═H/H and Y═O.

Compounds of Formula 1 are referred to as CRX-601 and CRX-527. Their structures are set forth as follows:

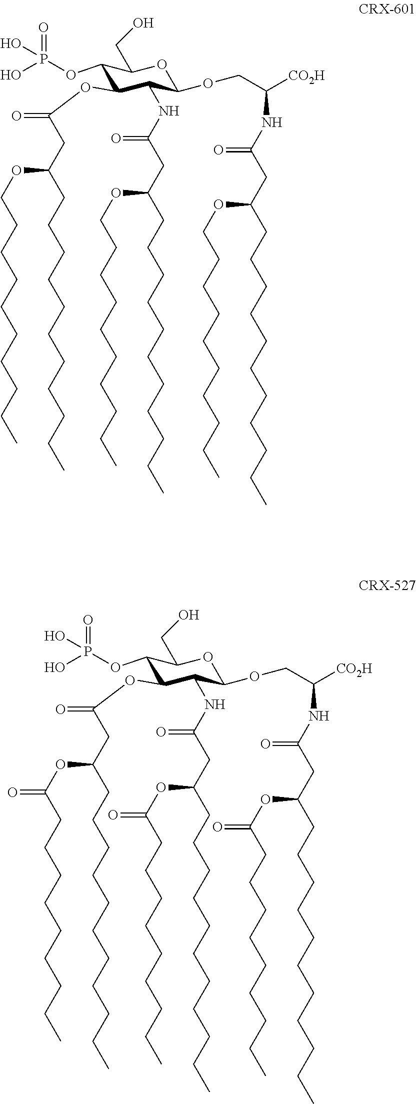

Additionally, another preferred embodiment employs CRX-547 having the structure shown. CRX-547

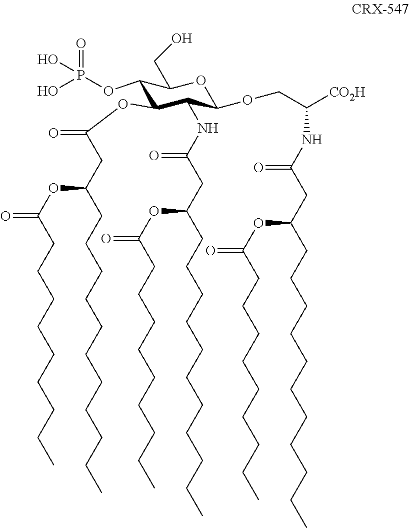

Still other embodiments include AGPs, such as CRX-602 or CRX-526 providing increased stability to AGPs having shorter secondary acyl or alkyl chains.

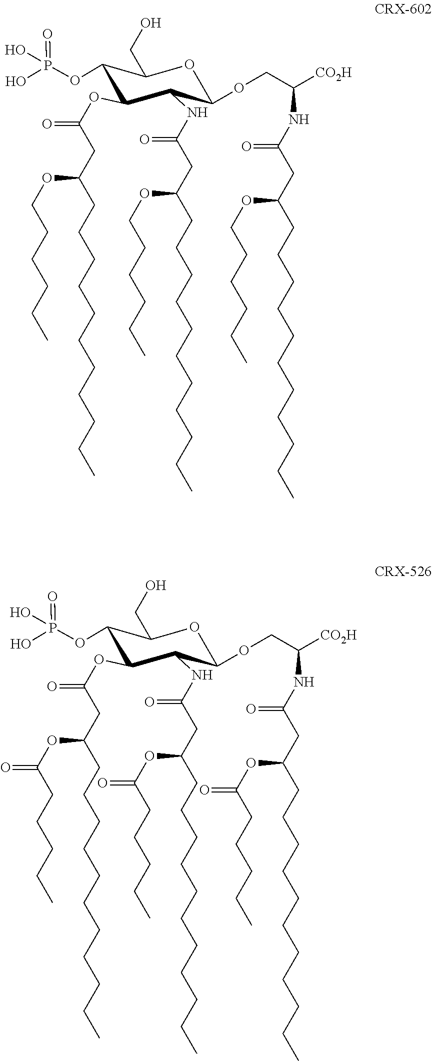

In a further embodiment of the invention, the TLR4 modulator is an agonist. In a further embodiment, the TLR4 modulator that is an agonist is selected from the group consisting of: CRX-601, CRX-547, and CRX-527.

### AGP Buffers

In one embodiment of the present invention, the composition comprising a TLR4 modulator, such as an AGP, is buffered using a zwitterionoic buffer. In one embodiment of the invention, the zwitterionic buffer is an aminoalkanesulfonic acid or suitable salt. Examples of amninoalkanesulfonic buffers include, but are not limited, to HEPES, HEPPS/EPPS, MOPS, MOBS and PIPES. In one embodiment of the invention, the buffer is a pharmaceutically acceptable buffer, suitable for use in humans, such as in for use in a commercial injection product. In one embodiment of the invention, the buffer is HEPES.

### Methods of Treatment

The combinations of the invention are believed to have utility in disorders wherein the engagement of OX40 and/or TLR4, is beneficial.

The present invention thus also provides a combination of the invention, for use in therapy, particularly, in the treatment of disorders wherein the engagement of OX40 and/or TLR4, is beneficial, particularly cancer.

In one embodiment, the present invention provides methods of treating cancer in a patient with the combination of a TLR4 agonist, such as CRX-601, with a humanized monoclonal OX40 antibody, wherein the humanized OX40 antibody is administered intravenously, and the TLR4 agonist is administered intratumorally, resulting in an abscopal effect in the tumor(s) in the patient.

As used herein, the term “abscopal effect”, means a phenomenon in which local treatment causes tumor regression at not only the treated site, but also at distant tumor sites. Postow, et al., N Engl J Med 366 (10): 925-31 (2012).

A further aspect of the invention provides a method of treatment of a disorder wherein engagement of OX40 and/or TLR4 is beneficial, comprising administering a combination of the invention.

A further aspect of the present invention provides the use of a combination of the invention in the manufacture of a medicament for the treatment of a disorder engagement of OX40 and/or TLR4 is beneficial. In some embodiments, the disorder is cancer. Suitably, the present invention provides the use of the combinations of the present invention for the treatment of cancer.

Examples of cancers that are suitable for treatment with combination of the invention include, but are limited to, both primary and metastatic forms of head and neck, breast, lung, colon, ovary, and prostate cancers. Suitably the cancer is selected from: brain (gliomas), glioblastomas, astrocytomas, glioblastoma multiforme, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, colon, head and neck, kidney, lung, liver, melanoma, ovarian, pancreatic, prostate, sarcoma, osteosarcoma, giant cell tumor of bone, thyroid, lymphoblastic T cell leukemia, Chronic myelogenous leukemia, Chronic lymphocytic leukemia, Hairy-cell leukemia, acute lymphoblastic leukemia, acute myelogenous leukemia, AML, Chronic neutrophilic leukemia, Acute lymphoblastic T cell leukemia, plasmacytoma, Immunoblastic large cell leukemia, Mantle cell leukemia, Multiple myeloma Megakaryoblastic leukemia, multiple myeloma, acute megakaryocytic leukemia, promyelocytic leukemia, Erythroleukemia, malignant lymphoma, hodgkins lymphoma, non-hodgkins lymphoma, lymphoblastic T cell lymphoma, Burkitt's lymphoma, follicular lymphoma, neuroblastoma, bladder cancer, urothelial cancer, lung cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor); and testicular cancer.

Additionally, examples of a cancer to be treated include Barret's adenocarcinoma; billiary tract carcinomas; breast cancer; cervical cancer; cholangiocarcinoma; central nervous system tumors including primary CNS tumors such as glioblastomas, astrocytomas (e.g., glioblastoma multiforme) and ependymomas, and secondary CNS tumors (i.e., metastases to the central nervous system of tumors originating outside of the central nervous system); colorectal cancer including large intestinal colon carcinoma; gastric cancer; carcinoma of the head and neck including squamous cell carcinoma of the head and neck; hematologic cancers including leukemias and lymphomas such as acute lymphoblastic leukemia, acute myelogenous leukemia (AML), myelodysplastic syndromes, chronic myelogenous leukemia, Hodgkin's lymphoma, non-Hodgkin's lymphoma, megakaryoblastic leukemia, multiple myeloma and erythroleukemia; hepatocellular carcinoma; lung cancer including small cell lung cancer and non-small cell lung cancer; ovarian cancer; endometrial cancer; pancreatic cancer; pituitary adenoma; prostate cancer; renal cancer; sarcoma; skin cancers including melanomas; and thyroid cancers.

Suitably, the present invention relates to a method for treating or lessening the severity of a cancer selected from: brain (gliomas), glioblastomas, astrocytomas, glioblastoma multiforme, Bannayan-Zonana syndrome, Cowden disease, Lhermitte-Duclos disease, breast, colon, head and neck, kidney, lung, liver, melanoma, ovarian, pancreatic, prostate, sarcoma and thyroid.

In one embodiment, the present invention relates to a method for treating or lessening the severity of a cancer selected from ovarian, breast, pancreatic and prostate.

In another embodiment, the present invention relates to a method for treating or lessening the severity of pre-cancerous syndromes in a mammal, including a human, wherein the pre-cancerous syndrome is selected from: cervical intraepithelial neoplasia, monoclonal gammapathy of unknown significance (MGUS), myelodysplastic syndrome, aplastic anemia, cervical lesions, skin nevi (pre-melanoma), prostatic intraepithleial (intraductal) neoplasia (PIN), Ductal Carcinoma in situ (DCIS), colon polyps and severe hepatitis or cirrhosis.

The combination of the invention may be used alone, or in combination with, one or more other therapeutic agents. The invention thus provides in a further aspect a further combination comprising a combination of the invention with a further therapeutic agent or agents, compositions and medicaments comprising the combination and use of the further combination, compositions and medicaments in therapy, in particular, in the treatment of diseases susceptible engagement of OX40 and/or TLR4.

In the embodiment, the combination of the invention may be employed with other therapeutic methods of cancer treatment. In particular, in anti-neoplastic therapy, combination therapy with other chemotherapeutic, hormonal, antibody agents as well as surgical and/or radiation treatments other than those mentioned above are envisaged.

Combination therapies according to the present invention thus include the administration of an anti-OX40 ABP or antibody of the invention and/or a TLR4 modulator as well as optional use of other therapeutic agents including other anti-neoplastic agents. Such combination of agents may be administered together or separately and, when administered separately this may occur simultaneously or sequentially in any order, both close and remote in time. In one embodiment, the pharmaceutical combination includes an anti-OX40 ABP or antibody of the invention and a TLR4 modulator, and optionally at least one additional anti-neoplastic agent.

In one embodiment, the further anti-cancer therapy is surgical and/or radiotherapy.

In one embodiment, the further anti-cancer therapy is at least one additional anti-neoplastic agent.

Any anti-neoplastic agent that has activity versus a susceptible tumor being treated may be utilized in the combination. Typical anti-neoplastic agents useful include, but are not limited to, anti-microtubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; non-receptor tyrosine angiogenesis inhibitors; immunotherapeutic agents; proapoptotic agents; and cell cycle signaling inhibitors.

Anti-microtubule or anti-mitotic agents: Anti-microtubule or anti-mitotic agents are phase specific agents active against the microtubules of tumor cells during M or the mitosis phase of the cell cycle. Examples of anti-microtubule agents include, but are not limited to, diterpenoids and vinca alkaloids.

Diterpenoids, which are derived from natural sources, are phase specific anti-cancer agents that operate at the G₂/M phases of the cell cycle. It is believed that the diterpenoids stabilize the β-tubulin subunit of the microtubules, by binding with this protein.

Disassembly of the protein appears then to be inhibited with mitosis being arrested and cell death following. Examples of diterpenoids include, but are not limited to, paclitaxel and its analog, docetaxel.

Paclitaxel, 5β,20-epoxy-1,2α,4,7β,10β,13α-hexa-hydroxytax-11-en-9-one 4,10-diacetate 2-benzoate 13-ester with (2R,3S)—N-benzoyl-3-phenylisoserine; is a natural diterpene product isolated from the Pacific yew tree Taxus brevifolia and is commercially available as an injectable solution TAXOL®. It is a member of the taxane family of terpenes. Paclitaxel has been approved for clinical use in the treatment of refractory ovarian cancer in the United States (Markman, et al., Yale Journal of Biology and Medicine, 64:583 (1991); McGuire, et al., Ann. Intem, Med., 111:273 (989), and for the treatment of breast cancer (Holmes, et al., J. Nat. Cancer Inst., 83:1797 (1991)). Paclitaxel is a potential candidate for treatment of neoplasms in the skin (Einzig, et. al., Proc. Am. Soc. Clin. Oncol., 20:46 (2001) and head and neck carcinomas (Forastire, et. al., Sem. Oncol., 20:56, (1990)). The compound also shows potential for the treatment of polycystic kidney disease (Woo, et. al., Nature, 368:750 (1994)), lung cancer and malaria. Treatment of patients with paclitaxel results in bone marrow suppression (multiple cell lineages, Ignoff, et. al, Cancer Chemotherapy Pocket Guide, 1998) related to the duration of dosing above a threshold concentration (50 nM) (Keams, et. al., Seminars in Oncology, 3(6) p. 16-23 (1995)).

Docetaxel, (2R,3S)—N-carboxy-3-phenylisoserine,N-tert-butyl ester, 13-ester with 5β-20-epoxy-1,2α,4,7β,10β,13α-hexahydroxytax-11-en-9-one 4-acetate 2-benzoate, trihydrate; is commercially available as an injectable solution as TAXOTERE®. Docetaxel is indicated for the treatment of breast cancer. Docetaxel is a semisynthetic derivative of paclitaxel q.v., prepared using a natural precursor, 10-deacetyl-baccatin III, extracted from the needle of the European Yew tree.

Vinca alkaloids are phase specific anti-neoplastic agents derived from the periwinkle plant. Vinca alkaloids act at the M phase (mitosis) of the cell cycle by binding specifically to tubulin. Consequently, the bound tubulin molecule is unable to polymerize into microtubules. Mitosis is believed to be arrested in metaphase with cell death following. Examples of vinca alkaloids include, but are not limited to, vinblastine, vincristine, and vinorelbine.

Vinblastine, vincaleukoblastine sulfate, is commercially available as VELBAN® as an injectable solution. Although, it has possible indication as a second line therapy of various solid tumors, it is primarily indicated in the treatment of testicular cancer and various lymphomas including Hodgkin's Disease; and lymphocytic and histiocytic lymphomas. Myelosuppression is the dose-limiting side effect of vinblastine.

Vincristine, vincaleukoblastine, 22-oxo-, sulfate, is commercially available as ONCOVIN® as an injectable solution. Vincristine is indicated for the treatment of acute leukemias and has also found use in treatment regimens for Hodgkin's and non-Hodgkin's malignant lymphomas. Alopecia and neurologic effects are the most common side effect of vincristine and to a lesser extent myelosupression and gastrointestinal mucositis effects occur.

Vinorelbine, 3′,4′-didehydro-4‘-deoxy-C’-norvincaleukoblastine [R—(R*,R*)-2,3-dihydroxybutanedioate (1:2)(salt)], commercially available as an injectable solution of vinorelbine tartrate (NAVELBINE®), is a semi-synthetic vinca alkaloid. Vinorelbine is indicated as a single agent or in combination with other chemotherapeutic agents, such as cisplatin, in the treatment of various solid tumors, such as non-small cell lung, advanced breast, and hormone refractory prostate cancers. Myelosuppression is the most common dose-limiting side effect of vinorelbine.

Platinum coordination complexes: Platinum coordination complexes are non-phase specific anti-cancer agents, which are interactive with DNA. The platinum complexes enter tumor cells, undergo, aquation and form intra- and interstrand cross-links with DNA causing adverse biological effects to the tumor. Examples of platinum coordination complexes include, but are not limited to, oxaliplatin, cisplatin and carboplatin.

Cisplatin, cis-diamminedichloroplatinum, is commercially available as PLATINOL® as an injectable solution. Cisplatin is primarily indicated in the treatment of metastatic testicular and ovarian cancer and advanced bladder cancer.

Carboplatin, platinum, diammine [1,1-cyclobutane-dicarboxylate(2-)-O,O′], is commercially available as PARAPLATIN® as an injectable solution. Carboplatin is primarily indicated in the first and second line treatment of advanced ovarian carcinoma.

Alkylating agents: Alkylating agents are non-phase anti-cancer specific agents and strong electrophiles. Typically, alkylating agents form covalent linkages, by alkylation, to DNA through nucleophilic moieties of the DNA molecule such as phosphate, amino, sulfhydryl, hydroxyl, carboxyl, and imidazole groups. Such alkylation disrupts nucleic acid function leading to cell death. Examples of alkylating agents include, but are not limited to, nitrogen mustards such as cyclophosphamide, melphalan, and chlorambucil; alkyl sulfonates such as busulfan; nitrosoureas such as carmustine; and triazenes such as dacarbazine.

Cyclophosphamide, 2-[bis(2-chloroethyl)amino]tetrahydro-2H-1,3,2-oxazaphosphorine 2-oxide monohydrate, is commercially available as an injectable solution or tablets as CYTOXAN®. Cyclophosphamide is indicated as a single agent, or in combination with other chemotherapeutic agents, in the treatment of malignant lymphomas, multiple myeloma, and leukemias.

Melphalan, 4-[bis(2-chloroethyl)amino]-L-phenylalanine, is commercially available as an injectable solution or tablets as ALKERAN®. Melphalan is indicated for the palliative treatment of multiple myeloma and non-resectable epithelial carcinoma of the ovary. Bone marrow suppression is the most common dose-limiting side effect of melphalan.

Chlorambucil, 4-[bis(2-chloroethyl)amino]benzenebutanoic acid, is commercially available as LEUKERAN® tablets. Chlorambucil is indicated for the palliative treatment of chronic lymphatic leukemia, and malignant lymphomas such as lymphosarcoma, giant follicular lymphoma, and Hodgkin's disease.

Busulfan, 1,4-butanediol dimethanesulfonate, is commercially available as MYLERAN® TABLETS. Busulfan is indicated for the palliative treatment of chronic myelogenous leukemia.

Carmustine, 1,3-[bis(2-chloroethyl)-1-nitrosourea, is commercially available as single vials of lyophilized material as BiCNU®. Carmustine is indicated for the palliative treatment as a single agent or in combination with other agents for brain tumors, multiple myeloma, Hodgkin's disease, and non-Hodgkin's lymphomas.

Dacarbazine, 5-(3,3-dimethyl-1-triazeno)-imidazole-4-carboxamide, is commercially available as single vials of material as DTIC-Dome®. Dacarbazine is indicated for the treatment of metastatic malignant melanoma and in combination with other agents for the second line treatment of Hodgkin's Disease.

Antibiotic anti-neoplastics: Antibiotic anti-neoplastics are non-phase specific agents, which bind or intercalate with DNA. Typically, such action results in stable DNA complexes or strand breakage, which disrupts ordinary function of the nucleic acids leading to cell death.

Examples of antibiotic anti-neoplastic agents include, but are not limited to, actinomycins such as dactinomycin, anthrocyclins such as daunorubicin and doxorubicin; and bleomycins.

Dactinomycin, also known as Actinomycin D, is commercially available in injectable form as COSMEGEN®. Dactinomycin is indicated for the treatment of Wilm's tumor and rhabdomyosarcoma.

Daunorubicin, (8S-cis-)-8-acetyl-10-[(3-amino-2,3,6-trideoxy-α-L-lyxo-hexopyranosyl)oxy]-7,8,9,10-tetrahydro-6,8,11-trihydroxy-1-methoxy-5,12 naphthacenedione hydrochloride, is commercially available as a liposomal injectable form as DAUNOXOME® or as an injectable as CERUBIDINE®. Daunorubicin is indicated for remission induction in the treatment of acute nonlymphocytic leukemia and advanced HIV associated Kaposi's sarcoma.

Doxorubicin, (8S, 10S)-10-[(3-amino-2,3,6-trideoxy-α-L-lyxo-hexopyranosyl)oxy]-8-glycoloyl, 7,8,9,10-tetrahydro-6,8,11-trihydroxy-1-methoxy-5,12 naphthacenedione hydrochloride, is commercially available as an injectable form as RUBEX® or ADRIAMYCIN RDF®. Doxorubicin is primarily indicated for the treatment of acute lymphoblastic leukemia and acute myeloblastic leukemia, but is also a useful component in the treatment of some solid tumors and lymphomas.

Bleomycin, a mixture of cytotoxic glycopeptide antibiotics isolated from a strain of Streptomyces verticillus, is commercially available as BLENOXANE®. Bleomycin is indicated as a palliative treatment, as a single agent or in combination with other agents, of squamous cell carcinoma, lymphomas, and testicular carcinomas.

Topoisomerase II inhibitors: Topoisomerase II inhibitors include, but are not limited to, epipodophyllotoxins.

Epipodophyllotoxins are phase specific anti-neoplastic agents derived from the mandrake plant. Epipodophyllotoxins typically affect cells in the S and G₂ phases of the cell cycle by forming a ternary complex with topoisomerase II and DNA causing DNA strand breaks. The strand breaks accumulate and cell death follows. Examples of epipodophyllotoxins include, but are not limited to, etoposide and teniposide.

Etoposide, 4′-demethyl-epipodophyllotoxin 9[4,6-0-(R)-ethylidene-β-D-glucopyranoside], is commercially available as an injectable solution or capsules as VePESID® and is commonly known as VP-16. Etoposide is indicated as a single agent, or in combination with, other chemotherapy agents in the treatment of testicular and non-small cell lung cancers.

Teniposide, 4′-demethyl-epipodophyllotoxin 9[4,6-0-(R)-thenylidene-β-D-glucopyranoside], is commercially available as an injectable solution as VUMON® and is commonly known as VM-26. Teniposide is indicated as a single agent or in combination with other chemotherapy agents in the treatment of acute leukemia in children.

Antimetabolite neoplastic agents: Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Examples of antimetabolite anti-neoplastic agents include, but are not limited to, fluorouracil, methotrexate, cytarabine, mecaptopurine, thioguanine, and gemcitabine.

5-fluorouracil, 5-fluoro-2,4-(1H,3H) pyrimidinedione, is commercially available as fluorouracil. Administration of 5-fluorouracil leads to inhibition of thymidylate synthesis and is also incorporated into both RNA and DNA. The result typically is cell death. 5-fluorouracil is indicated as a single agent or in combination with other chemotherapy agents in the treatment of carcinomas of the breast, colon, rectum, stomach and pancreas.

Other fluoropyrimidine analogs include 5-fluoro deoxyuridine (floxuridine) and 5-fluorodeoxyuridine monophosphate.

Cytarabine, 4-amino-1-β-D-arabinofuranosyl-2 (1H)-pyrimidinone, is commercially available as CYTOSAR-U® and is commonly known as Ara-C. It is believed that cytarabine exhibits cell phase specificity at S-phase by inhibiting DNA chain elongation by terminal incorporation of cytarabine into the growing DNA chain. Cytarabine is indicated as a single agent or in combination with other chemotherapy agents in the treatment of acute leukemia. Other cytidine analogs include 5-azacytidine and 2′,2′-difluorodeoxycytidine (gemcitabine).

Mercaptopurine, 1,7-dihydro-6H-purine-6-thione monohydrate, is commercially available as PURINETHOL®. Mercaptopurine exhibits cell phase specificity at S-phase by inhibiting DNA synthesis by an as of yet unspecified mechanism. Mercaptopurine is indicated as a single agent or in combination with other chemotherapy agents in the treatment of acute leukemia. A useful mercaptopurine analog is azathioprine.

Thioguanine, 2-amino-1,7-dihydro-6H-purine-6-thione, is commercially available as TABLOID®. Thioguanine exhibits cell phase specificity at S-phase by inhibiting DNA synthesis by an as of yet unspecified mechanism. Thioguanine is indicated as a single agent or in combination with other chemotherapy agents in the treatment of acute leukemia. Other purine analogs include pentostatin, erythrohydroxynonyladenine, fludarabine phosphate, and cladribine.

Gemcitabine, 2′-deoxy-2′,2′-difluorocytidine monohydrochloride (β-isomer), is commercially available as GEMZAR®. Gemcitabine exhibits cell phase specificity at S-phase and by blocking progression of cells through the G1/S boundary. Gemcitabine is indicated in combination with cisplatin in the treatment of locally advanced non-small cell lung cancer and alone in the treatment of locally advanced pancreatic cancer.

Methotrexate, N-[4[[(2,4-diamino-6-pteridinyl) methyl]methylamino] benzoyl]-L-glutamic acid, is commercially available as methotrexate sodium. Methotrexate exhibits cell phase effects specifically at S-phase by inhibiting DNA synthesis, repair and/or replication through the inhibition of dyhydrofolic acid reductase which is required for synthesis of purine nucleotides and thymidylate. Methotrexate is indicated as a single agent or in combination with other chemotherapy agents in the treatment of choriocarcinoma, meningeal leukemia, non-Hodgkin's lymphoma, and carcinomas of the breast, head, neck, ovary and bladder.

Topoisomerase I inhibitors: Camptothecins, including, camptothecin and camptothecin derivatives are available or under development as Topoisomerase I inhibitors. Camptothecins cytotoxic activity is believed to be related to its Topoisomerase I inhibitory activity. Examples of camptothecins include, but are not limited to, irinotecan, topotecan, and the various optical forms of 7-(4-methylpiperazino-methylene)-10,11-ethylenedioxy-20-camptothecin described below.

Irinotecan HCl, (4S)-4,11-diethyl-4-hydroxy-9-[(4-piperidinopiperidino) carbonyloxy]-1H-pyrano[3′,4′,6,7]indolizino[1,2-b]quinoline-3,14(4H,12H)-dione hydrochloride, is commercially available as the injectable solution CAMPTOSAR®. Irinotecan is a derivative of camptothecin which binds, along with its active metabolite SN-38, to the topoisomerase I-DNA complex. It is believed that cytotoxicity occurs as a result of irreparable double strand breaks caused by interaction of the topoisomerase I: DNA: irintecan or SN-38 ternary complex with replication enzymes. Irinotecan is indicated for treatment of metastatic cancer of the colon or rectum.

Topotecan HCl, (S)-10-[(dimethylamino)methyl]-4-ethyl-4,9-dihydroxy-1H-pyrano[3′,4′,6,7]indolizino[1,2-b]quinoline-3,14-(4H,12H)-dione monohydrochloride, is commercially available as the injectable solution HYCAMTIN®. Topotecan is a derivative of camptothecin which binds to the topoisomerase I-DNA complex and prevents religation of singles strand breaks caused by Topoisomerase I in response to torsional strain of the DNA molecule. Topotecan is indicated for second line treatment of metastatic carcinoma of the ovary and small cell lung cancer.

Hormones and hormonal analogues: Hormones and hormonal analogues are useful compounds for treating cancers in which there is a relationship between the hormone(s) and growth and/or lack of growth of the cancer. Examples of hormones and hormonal analogues useful in cancer treatment include, but are not limited to, adrenocorticosteroids such as prednisone and prednisolone which are useful in the treatment of malignant lymphoma and acute leukemia in children; aminoglutethimide and other aromatase inhibitors such as anastrozole, letrazole, vorazole, and exemestane useful in the treatment of adrenocortical carcinoma and hormone dependent breast carcinoma containing estrogen receptors; progestrins such as megestrol acetate useful in the treatment of hormone dependent breast cancer and endometrial carcinoma; estrogens, androgens, and anti-androgens such as flutamide, nilutamide, bicalutamide, cyproterone acetate and 5α-reductases such as finasteride and dutasteride, useful in the treatment of prostatic carcinoma and benign prostatic hypertrophy; anti-estrogens such as tamoxifen, toremifene, raloxifene, droloxifene, iodoxyfene, as well as selective estrogen receptor modulators (SERMS) such those described in U.S. Pat. Nos. 5,681,835, 5,877,219, and 6,207,716, useful in the treatment of hormone dependent breast carcinoma and other susceptible cancers; and gonadotropin-releasing hormone (GnRH) and analogues thereof which stimulate the release of leutinizing hormone (LH) and/or follicle stimulating hormone (FSH) for the treatment prostatic carcinoma, for instance, LHRH agonists and antagagonists such as goserelin acetate and luprolide.

Signal transduction pathway inhibitors: Signal transduction pathway inhibitors are those inhibitors, which block or inhibit a chemical process which evokes an intracellular change. As used herein this change is cell proliferation or differentiation. Signal tranduction inhibitors useful in the present invention include, but are not limited to, inhibitors of receptor tyrosine kinases, non-receptor tyrosine kinases, SH2/SH3 domain blockers, serine/threonine kinases, phosphotidyl inositol-3 kinases, myo-inositol signaling, and Ras oncogenes.

Several protein tyrosine kinases catalyse the phosphorylation of specific tyrosyl residues in various proteins involved in the regulation of cell growth. Such protein tyrosine kinases can be broadly classified as receptor or non-receptor kinases.

Receptor tyrosine kinases are transmembrane proteins having an extracellular ligand binding domain, a transmembrane domain, and a tyrosine kinase domain. Receptor tyrosine kinases are involved in the regulation of cell growth and are generally termed growth factor receptors. Inappropriate or uncontrolled activation of many of these kinases, i.e., aberrant kinase growth factor receptor activity, for example by over-expression or mutation, has been shown to result in uncontrolled cell growth. Accordingly, the aberrant activity of such kinases has been linked to malignant tissue growth. Consequently, inhibitors of such kinases could provide cancer treatment methods. Growth factor receptors include, for example, epidermal growth factor receptor (EGFr), platelet derived growth factor receptor (PDGFr), erbB2, erbB4, ret, vascular endothelial growth factor receptor (VEGFr), tyrosine kinase with immunoglobulin-like and epidermal growth factor identity domains (TIE-2), insulin growth factor-I (IGFI) receptor, macrophage colony stimulating factor (cfms), BTK, ckit, cmet, fibroblast growth factor (FGF) receptors, Trk receptors (TrkA, TrkB, and TrkC), ephrin (eph) receptors, and the RET protooncogene. Several inhibitors of growth receptors are under development and include ligand antagonists, antibodies, tyrosine kinase inhibitors and anti-sense oligonucleotides. Growth factor receptors and agents that inhibit growth factor receptor function are described, for instance, in Kath, John C., Exp. Opin. Ther. Patents (2000) 10(6):803-818; Shawver, et al DDT, Vol 2, No. 2 (February 1997); and Lofts, F. J., et al, GROWTH FACTOR RECEPTORS AS TARGETS”, NEW MOLECULAR TARGETS FOR CANCER CHEMOTHERAPY (Workman, Paul and Kerr, David, CRC press 1994, London).

Tyrosine kinases, which are not growth factor receptor kinases are termed non-receptor tyrosine kinases. Non-receptor tyrosine kinases useful in the present invention, which are targets or potential targets of anti-cancer drugs, include cSrc, Lck, Fyn, Yes, Jak, cAbl, FAK (Focal adhesion kinase), Brutons tyrosine kinase, and Bcr-Abl. Such non-receptor kinases and agents which inhibit non-receptor tyrosine kinase function are described in Sinh, et al., Journal of Hematotherapy and Stem Cell Research, 8 (5): 465-80 (1999); and Bolen, et al., Annual review of Immunology, 15: 371-404 (1997).

SH2/SH3 domain blockers are agents that disrupt SH2 or SH3 domain binding in a variety of enzymes or adaptor proteins including, PI3-K p85 subunit, Src family kinases, adaptor molecules (Shc, Crk, Nck, Grb2) and Ras-GAP. SH2/SH3 domains as targets for anti-cancer drugs are discussed in Smithgall, T. E., Journal of Pharmacological and Toxicological Methods, 34(3) 125-32 (1995).

Inhibitors of Serine/Threonine Kinases including MAP kinase cascade blockers which include blockers of Raf kinases (rafk), Mitogen or Extracellular Regulated Kinase (MEKs), and Extracellular Regulated Kinases (ERKs); and Protein kinase C family member blockers including blockers of PKCs (alpha, beta, gamma, epsilon, mu, lambda, iota, zeta). IkB kinase family (IKKa, IKKb), PKB family kinases, akt kinase family members, and TGF beta receptor kinases. Such Serine/Threonine kinases and inhibitors thereof are described in Yamamoto, et al., Journal of Biochemistry, 126 (5) 799-803 (1999); Brodt, et al., Biochemical Pharmacology, 60. 1101-1107 (2000); Massague, et al., Cancer Surveys, 27:41-64 (1996); Philip, et al., Cancer Treatment and Research, 78: 3-27 (1995), Lackey, et al., Bioorganic and Medicinal Chemistry Letters, (10) 223-226 (2000); U.S. Pat. No. 6,268,391; and Martinez-Iacaci, et al, Int. J. Cancer, 88(1), 44-52 (2000).

Inhibitors of Phosphotidyl inositol-3 Kinase family members including blockers of PI3-kinase, ATM, DNA-PK, and Ku are also useful in the present invention. Such kinases are discussed in Abraham, R. T. (1996), Current Opinion in Immunology. 8 (3) 412-8; Canman, C. E., Lim, D. S. (1998), Oncogene 17 (25) 3301-3308; Jackson, S. P. (1997), International Journal of Biochemistry and Cell Biology. 29 (7):935-8; and Zhong, H., et al, Cancer Res., (2000) 60(6), 1541-1545.

Also useful in the present invention are myo-inositol signaling inhibitors, such as phospholipase C blockers and Myoinositol analogues. Such signal inhibitors are described in Powis, G., and Kozikowski A., (1994) NEW MOLECULAR TARGETS FOR CANCER CHEMOTHERAPY ED. (Paul Workman and David Kerr, CRC press 1994, London).

Another group of signal transduction pathway inhibitors are inhibitors of Ras Oncogene. Such inhibitors include inhibitors of famesyltransferase, geranyl-geranyl transferase, and CAAX proteases as well as anti-sense oligonucleotides, ribozymes and immunotherapy. Such inhibitors have been shown to block ras activation in cells containing wild-type mutant ras, thereby acting as antiproliferation agents. Ras oncogene inhibition is discussed in Scharovsky, et al. (2000), Journal of Biomedical Science. 7(4) 292-8; Ashby, M. N. (1998), Current Opinion in Lipidology. 9 (2) 99-102; and Bio Chim. Biophys. Acta, (1989) 1423(3):19-30.

As mentioned above, antibody antagonists to receptor kinase ligand binding may also serve as signal transduction inhibitors. This group of signal transduction pathway inhibitors includes the use of humanized antibodies to the extracellular ligand binding domain of receptor tyrosine kinases. For example, Imclone C225 EGFR specific antibody (see Green, et al, Monoclonal Antibody Therapy for Solid Tumors, Cancer Treat. Rev., (2000), 26(4), 269-286); Herceptin® erbB2 antibody (see “Tyrosine Kinase Signalling in Breast cancer:erbB Family Receptor Tyrosine Kinases”, Breast Cancer Res., 2000, 2(3), 176-183); and 2CB VEGFR2 specific antibody (see Brekken, et al., “Selective Inhibition of VEGFR2 Activity by a monoclonal Anti-VEGF antibody blocks tumor growth in mice”, Cancer Res. (2000) 60, 5117-5124).

Anti-angiogenic agents: Anti-angiogenic agents including non-receptor MEKngiogenesis inhibitors may also be useful. Anti-angiogenic agents such as those which inhibit the effects of vascular endothelial growth factor, (for example the anti-vascular endothelial cell growth factor antibody bevacizumab [Avastin™], and compounds that work by other mechanisms (for example linomide, inhibitors of integrin αvβ3 function, endostatin and angiostatin); Immunotherapeutic agents: Agents used in immunotherapeutic regimens may also be useful in combination with the compounds of formula (I). Immunotherapy approaches, including for example ex-vivo and in-vivo approaches to increase the immunogenicity of patient tumor cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumor cell lines and approaches using anti-idiotypic antibodies

Proapoptotoc agents: Agents used in proapoptotic regimens (e.g., bcl-2 antisense oligonucleotides) may also be used in the combination of the present invention.

Cell cycle signaling inhibitors: Cell cycle signaling inhibitors inhibit molecules involved in the control of the cell cycle. A family of protein kinases called cyclin dependent kinases (CDKs) and their interaction with a family of proteins termed cyclins controls progression through the eukaryotic cell cycle. The coordinate activation and inactivation of different cyclin/CDK complexes is necessary for normal progression through the cell cycle. Several inhibitors of cell cycle signaling are under development. For instance, examples of cyclin dependent kinases, including CDK2, CDK4, and CDK6 and inhibitors for the same are described in, for instance, Rosania, et al., Exp. Opin. Ther. Patents (2000) 10(2):215-230.

In one embodiment, the combination of the present invention comprises an anti-OX40 ABP or antibody and a TLR4 modulator and at least one anti-neoplastic agent selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine MEKngiogenesis inhibitors, immunotherapeutic agents, proapoptotic agents, and cell cycle signaling inhibitors.

In one embodiment, the combination of the present invention comprises an anti-OX40 ABP or antibody and a TLR4 modulator and at least one anti-neoplastic agent which is an anti-microtubule agent selected from diterpenoids and vinca alkaloids.

In a further embodiment, the anti-neoplastic agent is a diterpenoid.

In a further embodiment, the anti-neoplastic agent is a vinca alkaloid.

In one embodiment, the combination of the present invention comprises an anti-OX40 ABP or antibody and a TLR4 modulator and at least one anti-neoplastic agent, which is a platinum coordination complex.

In a further embodiment, the anti-neoplastic agent is paclitaxel, carboplatin, or vinorelbine.

In one embodiment, the combination of the present invention comprises an anti-OX40 ABP or antibody and a TLR4 modulator and at least one anti-neoplastic agent which is a signal transduction pathway inhibitor.

In a further embodiment, the signal transduction pathway inhibitor is an inhibitor of a growth factor receptor kinase, VEGFR2, TIE2, PDGFR, BTK, erbB2, EGFr, IGFR-1, TrkA, TrkB, TrkC, or c-fms.

In a further embodiment, the signal transduction pathway inhibitor is an inhibitor of a serine/threonine kinase rafk, akt, or PKC-zeta.

In a further embodiment, the signal transduction pathway inhibitor is an inhibitor of a non-receptor tyrosine kinase selected from the src family of kinases.

In a further embodiment, the signal transduction pathway inhibitor is an inhibitor of c-src.

In a further embodiment, the signal transduction pathway inhibitor is an inhibitor of Ras oncogene selected from inhibitors of famesyl transferase and geranylgeranyl transferase.

In a further embodiment, the signal transduction pathway inhibitor is an inhibitor of a serine/threonine kinase selected from the group consisting of PI3K.

In a further embodiment, the signal transduction pathway inhibitor is a dual EGFr/erbB2 inhibitor, for example N-{3-Chloro-4-[(3-fluorobenzyl) oxy]phenyl}-6-[5-({[2-(methanesulphonyl) ethyl]amino}methyl)-2-furyl]-4-quinazolinamine (structure below):

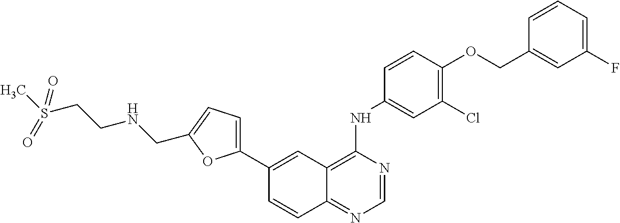 [SMILES: CS(=O)(=O)CCNCc1ccc(-c2ccc3ncnc(Nc4ccc(OCc5cccc(F)c5)c(Cl)c4)c3c2)o1]

In one embodiment, the combination of the present invention comprises a compound of formula I or a salt or solvate thereof and at least one anti-neoplastic agent which is a cell cycle signaling inhibitor.

In further embodiment, cell cycle signaling inhibitor is an inhibitor of CDK2, CDK4, or CDK6.

In one embodiment the mammal in the methods and uses of the present invention is a human.

As indicated, therapeutically effective amounts of the combinations of the invention (an anti-OX40 ABP or antibody and a TLR4 modulator) are administered to a human. Typically, the therapeutically effective amount of the administered agents of the present invention will depend upon a number of factors including, for example, the age and weight of the subject, the precise condition requiring treatment, the severity of the condition, the nature of the formulation, and the route of administration. Ultimately, the therapeutically effective amount will be at the discretion of the attendant physician.

The following examples are intended for illustration only, and are not intended to limit the scope of the invention in any way.

### Examples

### Example 1: Treatment of OX86 Monotherapy in a CT-26 Syngeneic Mouse Model for Colon Cancer

The CT26 mouse colon carcinoma (CT26.WT; ATCC #CRL-2638) cell line was obtained from ATCC. It is an N-nitroso-N-methylurethane-(NNMU) induced, undifferentiated colon carcinoma cell line known in the art. For example, it is described in: Wang M, et al. Active immunotherapy of cancer with a nonreplicating recombinant fowlpox virus encoding a model tumor-associated antigen. J. Immunol. 154: 4685-4692, 1995 (PubMed: 7722321). Rat IgG1 was obtained from Bioxcell. OX86 (Hybridoma 134) cells were obtained from the European Cell Culture collection and manufactured by Harlan; OX86 is the name for a tool anti-OX40 monoclonal antibody used in rodents; it is a rodent antibody that binds rodent OX40, e.g., mouse OX40 (receptor).

OX86 and rat IgG1 were diluted in diluted DPBS.

For preparation of tumor cells, a frozen (−140° C.) vial of CT-26 (mouse colon carcinoma cells), from ATCC (cat# CRL-2638, lot#59227052) were thawed and cultured in basic RPMI (with 10% FBS) media over the following week.

CT-26 cells (passage 12) were harvested from the flask in complete medium. Cells were centrifuged and resuspended in RPMI (without FBS), this step is repeated 3 times. Cell density and viability were checked via trypan blue exclusion. Cells were then diluted to desired density (5×10⁵ cells per mL) and kept on ice.

Escalating doses of OX40 monoclonal antibody (mAb) OX86 were evaluated for their efficacy in reducing tumor growth. Animals were weighed and innoculated on the right hind quarter with 0.5×10⁵ CT26 tumor cells per mouse on Day 0. A total of 130 mice were inoculated with tumor cells—assuming 30% failure rate (either too big or too small at time of start of study), the goal was to have n=10 for each group. After tumor cell inoculation, tumor growth and total body weight are measured 3 times a week for the duration of the study. Randomization occurred on day 10 or 11 when the average tumor volume was approximately 100 mm³. Beginning on the day of randomization, animals were dosed with OX86 mAb or Rat IgG1 isotype i.p. biweekly, for a total of 6 doses. Mice remained on study until tumors reach >2000 cu mm for two consecutive measurements, they were removed from study for other reasons (i.e., weight loss >20%, ulceration on tumor, etc.) or until the end of the study. After euthanization the tumors were removed and subject to dissociation for flow analysis and/or FFPE for IHC analysis.

| | | | No. of |
|---|---|---|---|
| | Treatment | Dose | mice |
| Group 1: | 0.5 × 10⁵ cells per, Rat | 400 ug per mouse | 10-13 |
| | IgG1 | | |
| Group 2: | 0.5 × 10⁵ cells per, OX86 | 400 ug per mouse | 10-13 |
| Group 3: | 0.5 × 10⁵ cells per, OX86 | 200 ug per mouse | 10-13 |
| Group 4: | 0.5 × 10⁵ cells per, OX86 | 100 ug per mouse | 10-13 |
| Group 5: | 0.5 × 10⁵ cells per, OX86 | 50 ug per mouse | 10-13 |

Day 0: sc innoculation with tumor cells
Days 1, 4, 6, 8: Animals were weighed and checked for tumors and
if present, tumors measured.
Randomization day (approx. day 10): Animals were randomized and
placed into cages representing appropriate groups
Dosing, biweekly through end of study: Animals were dosed ip
with OX86 or
anti Rat IgG1, where the amounts shown above were on a per
mouse basis.
Measurements, triweekly through end of study: Animals were
weighed and tumors were measured

The mean tumor weights from about 10 animals were averaged. Error bars show SEM analysis. P values were calculated based on the following: P value tested the null hypothesis that the survival curves were identical in the overall populations. In other words, the null hypothesis is that the treatment did not change survival. Raw p-values adjusted for multiple comparisons via the Stepdown Bonferroni method

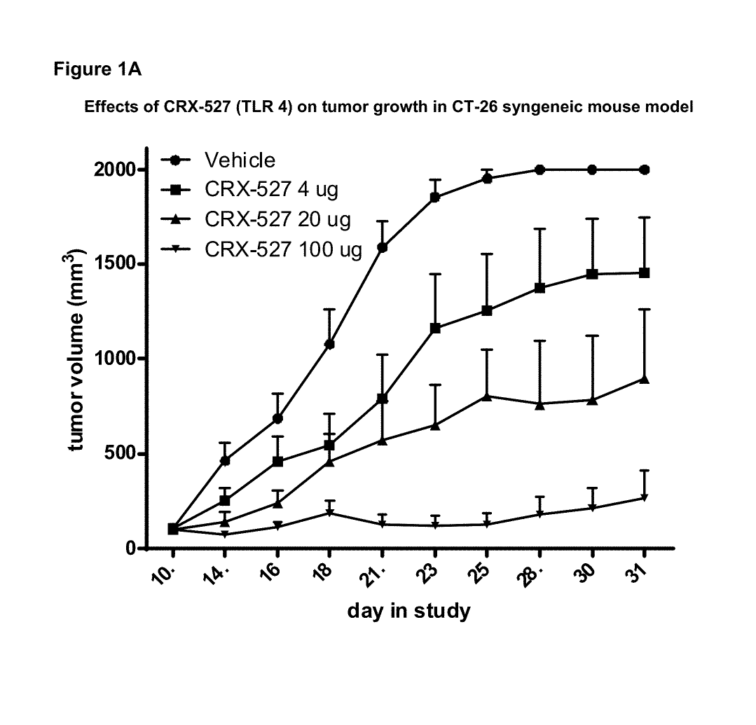

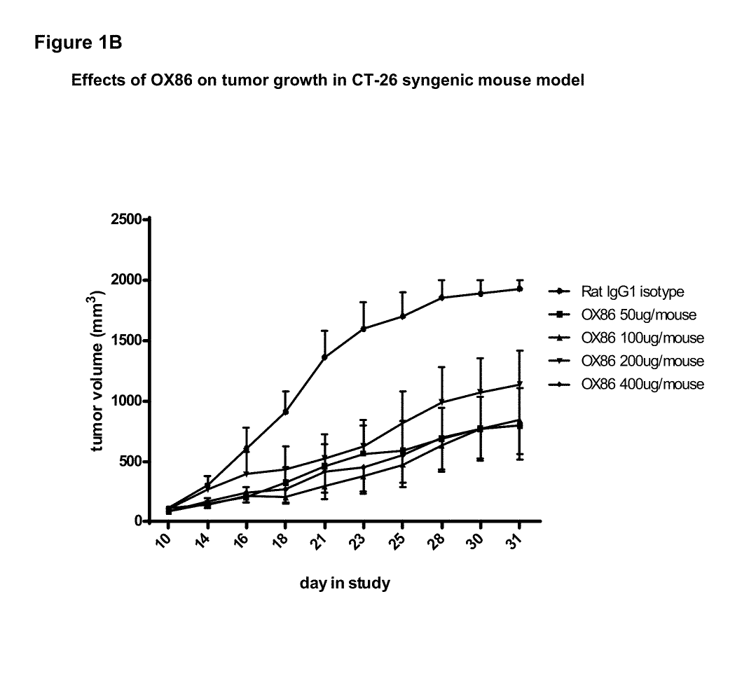

The above protocol was used to generate the results in FIG. 1B, and results of the individual mice can be found in FIG. 4. These figures demonstrate that mice inoculated with CT-26 cells and treated with rat IgG1 developed tumors that grew unabated as expected, whereas dosing with OX40 monoclonal antibody (mAb) OX86 led to clear inhibition of tumor growth and increased survivability when compared to the rat IgG1 control group.

### Example 2: Results of CT-26 Study with Treatment with TLR4 (CRX-527)

The addition of TLR4 modulators such as CRX-527 to the above OX40 monotherapy treatment protocol were used to study TLR4 monotherapy and the combination of anti-mOX40 immunotherapy with TLR4 modulators.

| | Treatment | Dose (per mouse) | No. of mice |
|---|---|---|---|
| Group 0: | 0.5 × 10⁵ cells per, | vehicle | 10-13 |
| Group a: | 0.5 × 10⁵ cells per, | CRX-527; 4 ug | 10-13 |
| Group b: | 0.5 × 10⁵ cells per, | CRX-527; 20 ug | 10-13 |
| Group c: | 0.5 × 10⁵ cells per, | CRX-527; 100 ug | 10-13 |

Day 0: sc innoculation with tumor cells
Days 1, 4, 6, 8: Animals were weighed and checked for tumors and
measured.
Randomization day (approx. day 10): Animals were randomized and
placed into cages representing appropriate groups
Dosing, biweekly through end of study: Animals dosed ip with TLR
compound CRX-527 at amounts shown above (per mouse), or vehicle.
Measurements, triweekly through end of study: Animals weighed and
tumors measured.

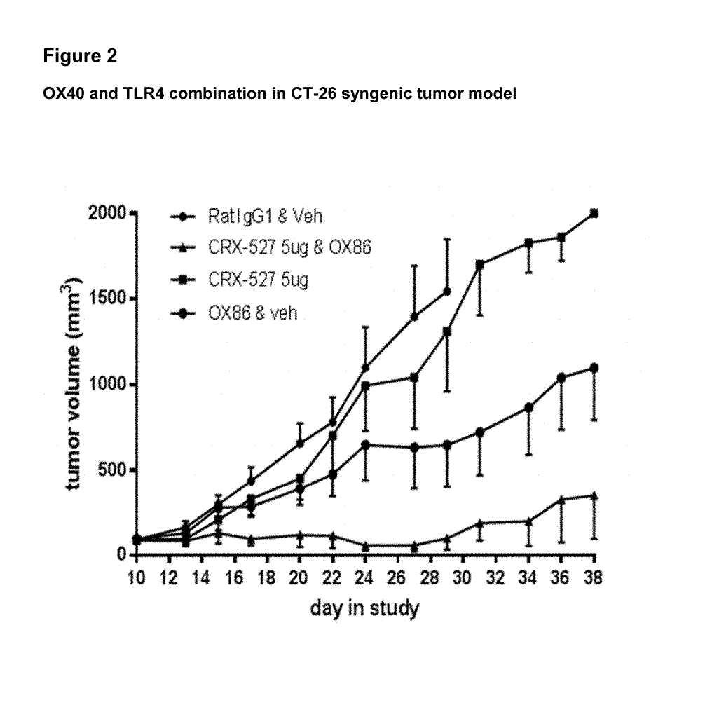

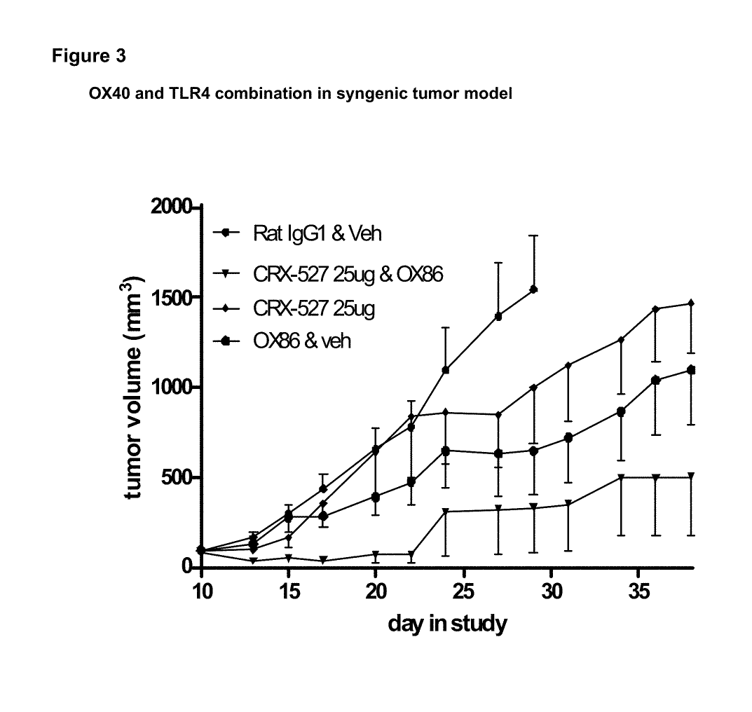

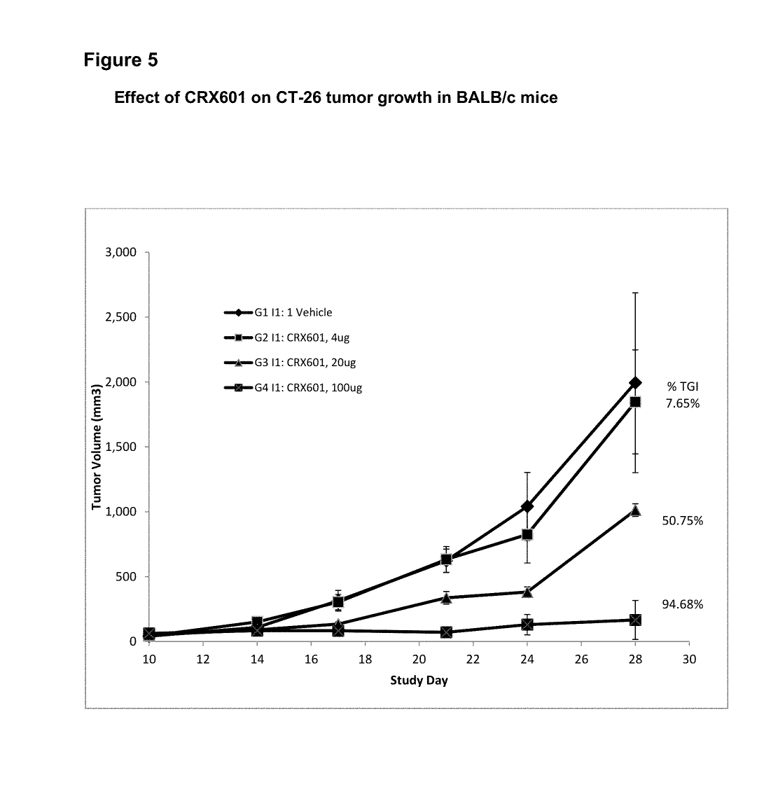

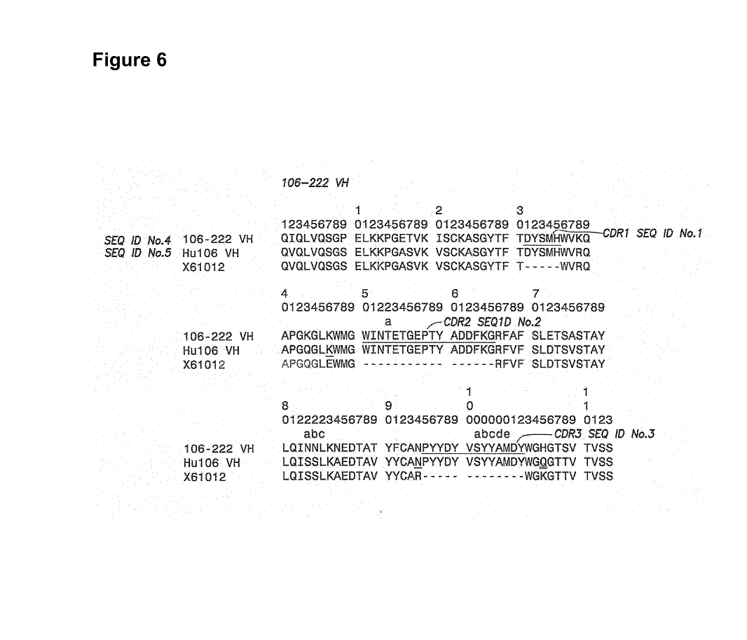

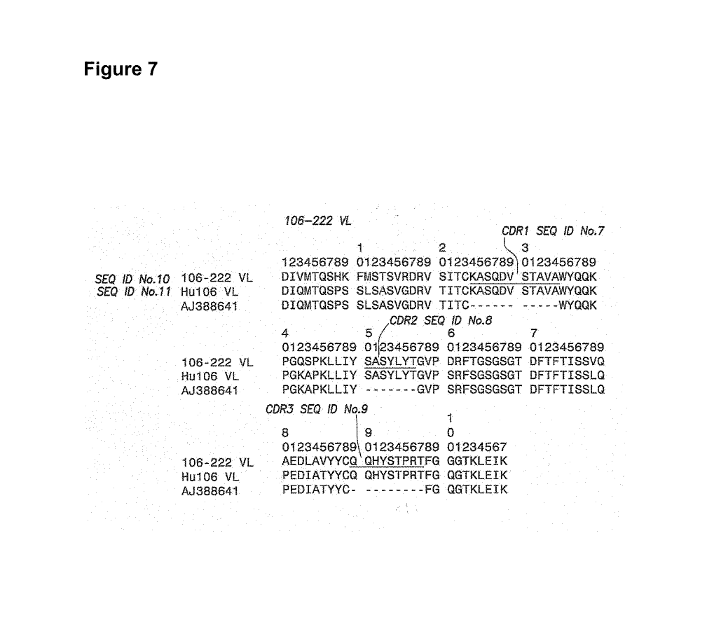

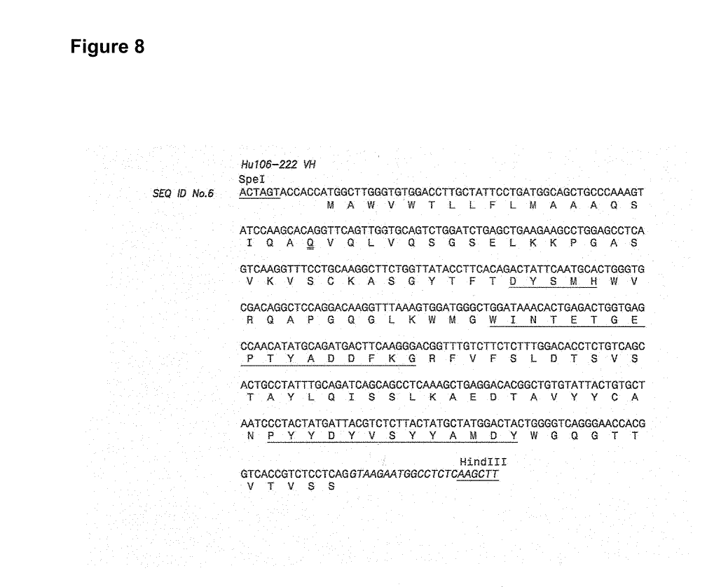

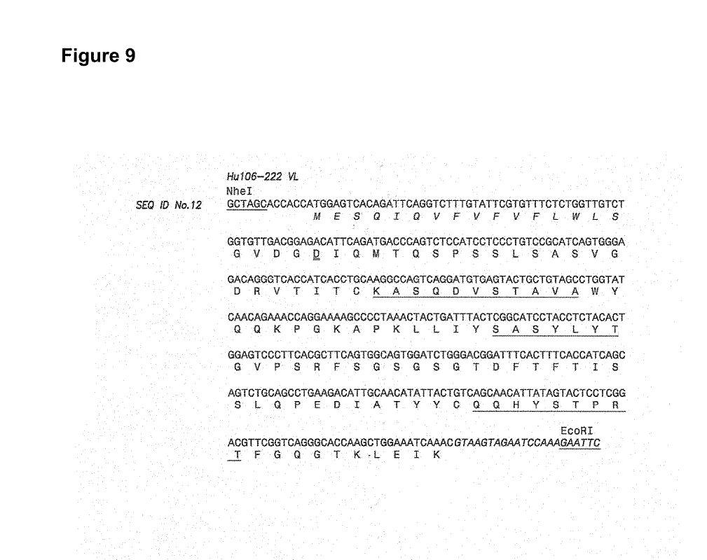

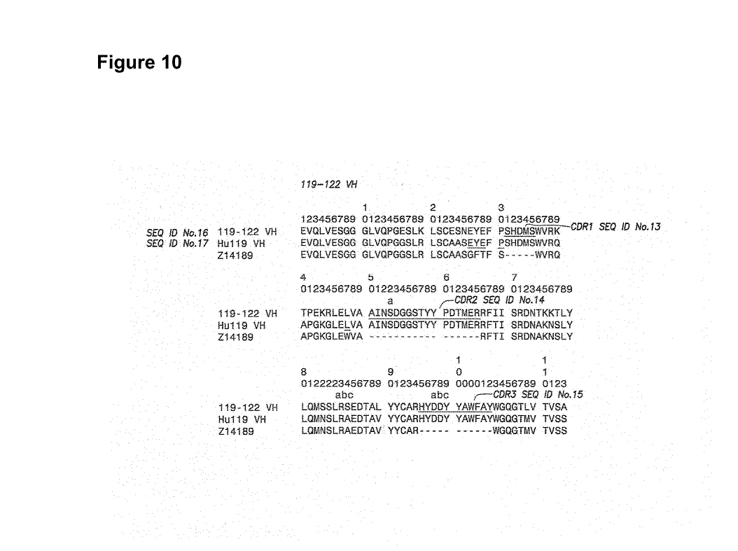

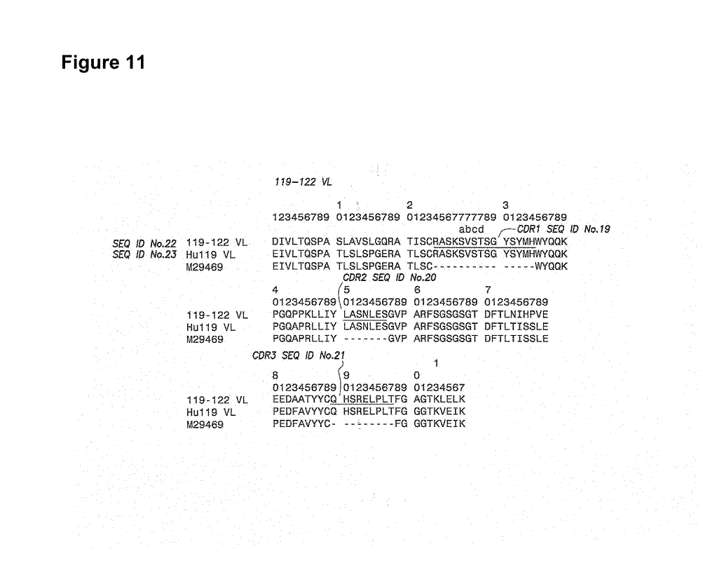

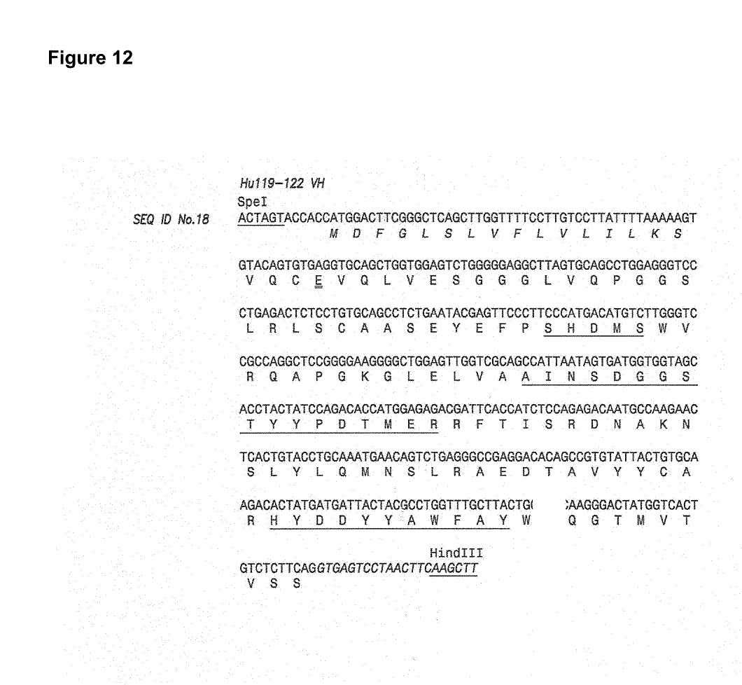

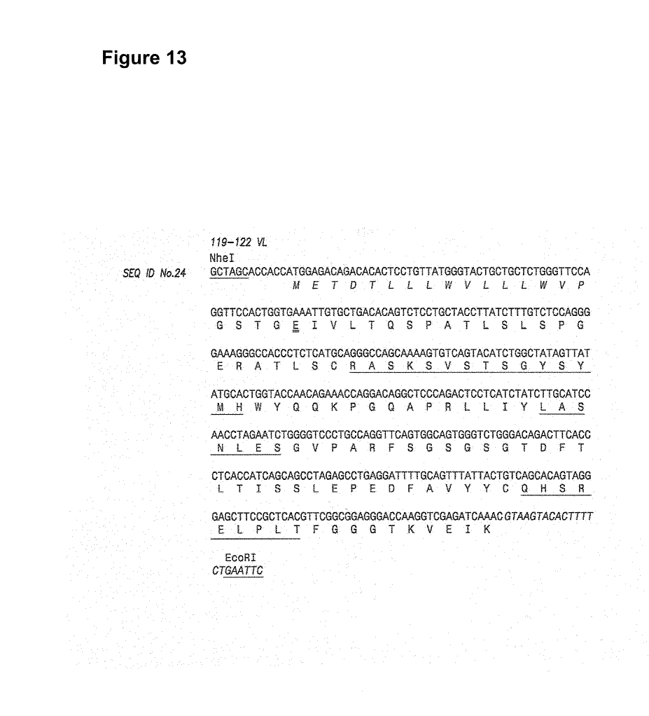

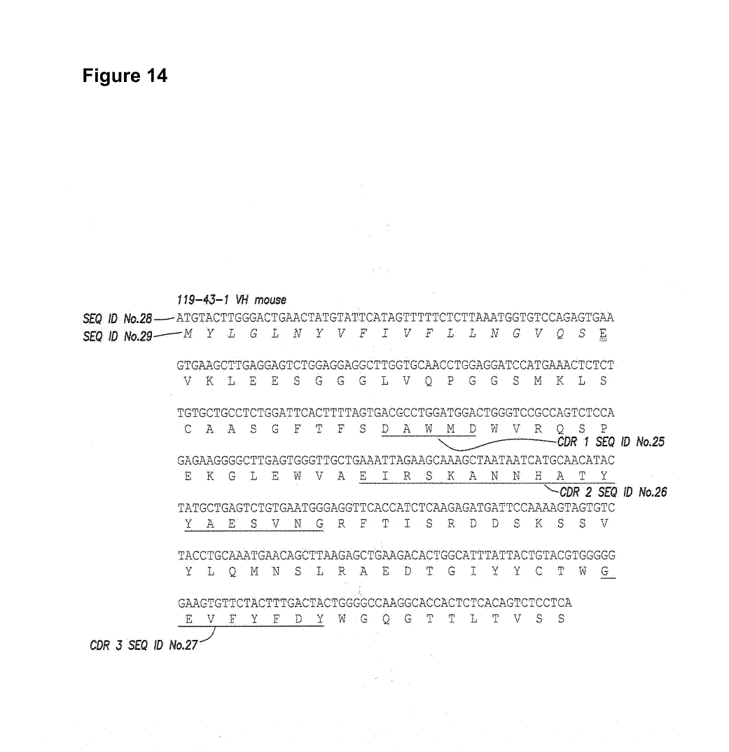

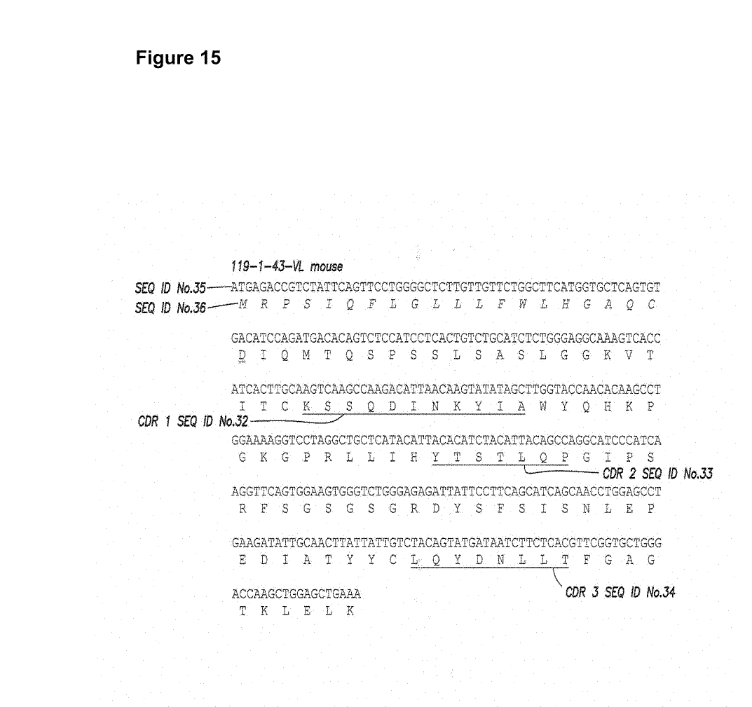

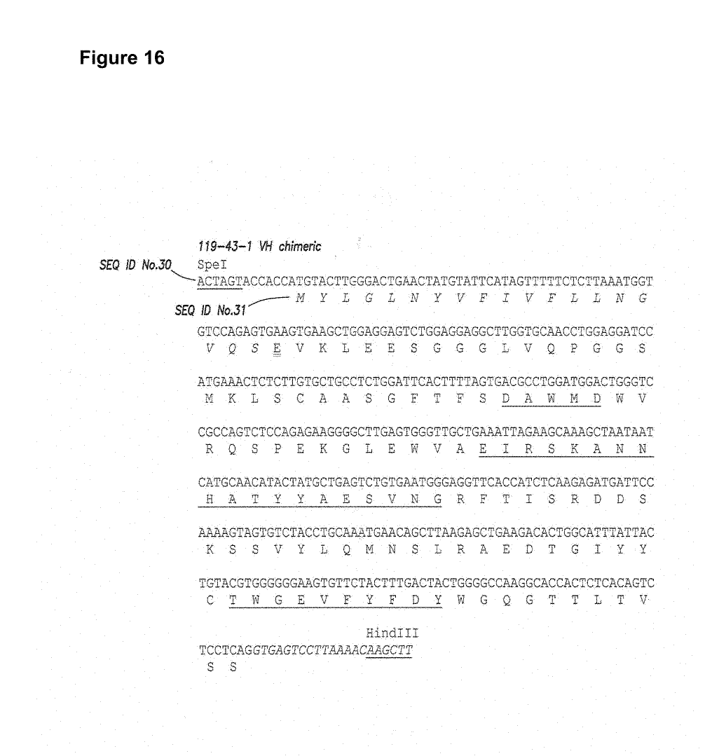

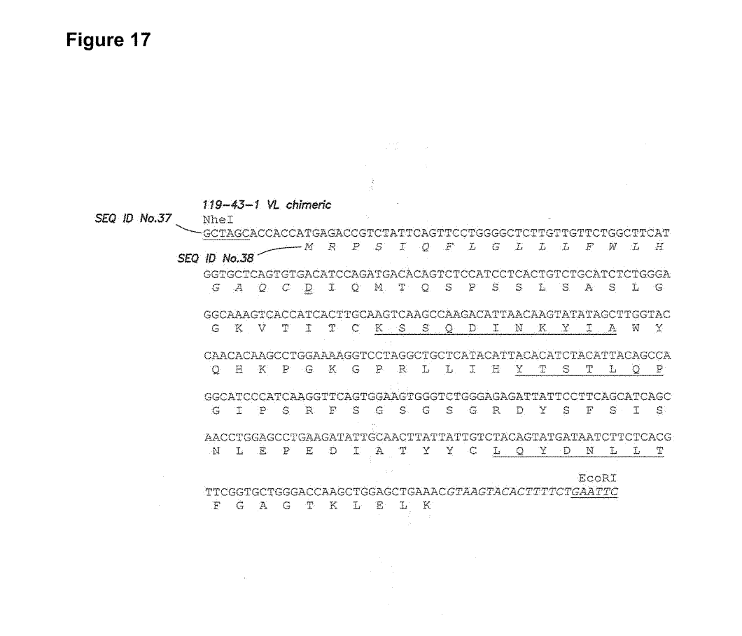

The above protocol was used to generate the results in FIG. 1A and FIGS. 2-6 at the dosages indicated. In almost every case, Balb/c mice that were inoculated with 0.5×10⁵ CT-26 colorectal tumor cells on the right hind quarter developed tumors that, when treated i.p. with vehicle (2% glycerol) only, and progressed as expected. TLR 4 agonists CRX-527 (FIGS. 2-5) and CRX-601 (FIG. 6) inhibited tumor growth in a dose-dependent manner when compared to the vehicle treated animals. Dose dependence was also seen in the survivability of the model.

### Example 3: Combination Treatment with OX40 (i.e., OX-86, an Antibody Raised Against Rodent OX40 Receptor) and CRX-527

The following treatment schedule was performed:

| | | treat- | | number |
|---|---|---|---|---|
| | Dosing | ment 1 | treatment 2 | of mice |
| Group 1: | 0.5 × 105 cells per, | Rat IgG1 | drug vehicle | 10-13 |
| Group 2: | 0.5 × 105 cells per, | OX86 50 | drug vehicle | 10-13 |
| | | ug | | |
| Group 3: | 0.5 × 105 cells per, | RatIgG1 | CRX-527 5 | 10-13 |
| | | | ug | |
| Group 4: | 0.5 × 105 cells per, | RatIgG1 | CRX-527 25 | 10-13 |
| | | | ug | |
| Group 5: | 0.5 × 105 cells per, | OX86 50 | CRX-527 5 | 10-13 |
| | | ug | ug | |
| Group 6: | 0.5 × 105 cells per, | OX86 50 | CRX-527 | 10-13 |
| | | ug | 25 ug | |

Day 0: SC innoculation with tumor cells
Days 1, 4, 6, 8: Animals checked for tumors and if present, tumors
measured.
Study enrollment day (approx. day 10): Animals randomized and
received treatment 1.
Biweekly post enrollment: starting with day of enrollment, mice
received i.p. dose biweekly for a total 6 doses.
Triweekly through end of study: Animals weighed and tumors
measured

When OX86 treatment was combined with TLR4 modulator treatment (CRX-527), mice exhibited a higher reduction in tumor burden and survived longer than either treatment alone.

### Example 4: Monotherapy and Combination Treatment with Anti-mOX40R Antibody and TLR4 Targeting Molecules of Formula I

Mice were administered OX40 antibody; a compound of Formula 1 (including a compound of Formula Ia, CRX-527, CRX-547, and CRX-601 (TLR4 agonists), or a combination of both. Each treatment has significant anti-tumor activity.

There are at least two significant findings. First, in mice, anti-OX40R or combination of anti-OX40 antibody and TLR4 agonist combination each delayed the growth of established CT-26 tumors relative to an untreated control group. Secondly, in mice significant anti-tumor effect was observed in TLR4 agonist and anti-OX40R antibody combinations as compared to monotherapy treatment.

### Example 5: Combination Treatment with an OX40R ABS (i.e., Anti-mOX40 Receptor Antibody Clone OX-86, an Antibody Raised Against Rodent OX40 Receptor) and CRX-601

### Materials and Methods

### In Vivo Anti-Tumor Efficacy Studies

The in vivo anti-tumor efficacy of the TLR4 agonist (CRX601) was assessed in the murine CT-26 colon carcinoma syngeneic solid tumor model as a monotherapy and in combination with a rate anti-mouse OX40 antibody clone OX86. Seven to eight week old female Balb/c mice (BALB/cAnNCrl, Charles River) were used in these studies. Murine CT-26 colon carcinoma cells (ATCC catalog number CRL-2638 lot#59227052) were cultured in RPMI growth medium supplemented with 10% fetal bovine serum (FBS) in a humidified 37° C. incubator with 5% CO₂. CT-26 cells cultured in logarithmic growth were harvested from tissue culture flasks and centrifuged for 5 minutes at 450×g at 4° C. for ten minutes to pellet cells. The supernatant was discarded, and cells were washed in ice cold phosphate buffered saline (PBS) without calcium and magnesium and centrifuged again for 5 minutes at 450×g at 4° C. for ten minutes to pellet cells. The cells were resuspended in sterile RPMI media without FBS and adjusted to a cell concentration of 500,000 cells/ml. 100 μl of the cell stock was implanted via subcutaneous injection into the right flank of each Balb/c mouse. After ten or eleven days when the average tumor size reached approximately 100 mm³, mice were randomized into study cohorts according to tumor size and the first treatment dose was given. The TLR4 agonist (CRX601) or vehicle was dosed via a systemic intravenous or direct intratumoral injection as indicated. The CRX-601 vehicle used for intravenous and intratumoral dosing was 0.5% where indicated. For CRX-601 liposomal intratumoral dosing, a DOPC/CHOL liposome prepared by GSK Lot #1783-157-B was used. The rat anti-mouse OX40 receptor antibody (clone OX86) (expressed and purified in-house from the rat hybridoma Grits ID 50776, BP232 2013) or Rat IgG1 isotype control antibody (BioXCell catalog # BE0088) was dosed via an intraperitoneal injection given twice per week for a total of six doses. Caliper measurements were taken three times per week to assess tumor growth, and mice with tumors <2,000 mm³ were maintained on study from 30 up to approximately 115 days. Mice with tumors >2,000 mm³ for 2 consecutive measurements or mice with tumors which formed open ulcers were removed from the study. Tumor volume was calculated using the formula (0.52)×(Length)×(Width²). In studies 6 and 7, tumor-free mice were re-challenged with CT-26 tumor cells as described above, on the opposite flank from the original inoculation site and tumor growth was monitored, as described above. All studies were conducted in accordance with the GSK Policy on the Care, Welfare and Treatment of Laboratory Animals and were reviewed by the Institutional Animal Care and Use Committee at GSK.

### Immunephenotyping and Cytokine Analysis

Tumors, blood and tissues were harvested from CT-26 mice on day 0, day 1 and day 8 after first CRX-601 dosing. Mouse white blood cells and dissociated tumor single cells were stained freshly with surface or intracellular staining antibodies for multicolor flow cytometry analysis for immunephenotyping. Multiplex cytokine analysis was performed using mouse plasma samples from the same study.

### Statistical Analysis

For studies 1-4, to determine significance of tumor growth inhibition, tumor volumes at 11 (study 1), 15 (studies 2 and 3), or 19 (study 4) days after first dose were compared between the different treatment groups. Prior to the analysis, tumor volumes were natural log transformed due to the inequality of variance in the different treatment groups. ANOVA followed by pair-wise comparison was then carried out on the log transformed data. SAS 9.3 and R 3.0.2 analysis software was used. Kaplan-Meier (KM) method was carried out to estimate the survival probability of different treatment groups at a given time. The event for survival analysis was tumor volume of 2000 mm³ or tumor ulceration, whichever came first. The exact time to cut-off volume was estimated by fitting a linear line between log tumor volume and day of two observations, the first observation that exceed the cut-off volume and the one observation that immediately preceded the cut-off volume. The median time to endpoint and its corresponding 95% confidence interval was calculated. Whether or not KM survival curves were statistically different between any two groups was then tested by log-rank test. The raw p-value, as well as the false discovery rate (FDR) adjusted p-values, from the comparisons of days to events by survival analysis and the comparisons of log transformed tumor volume at indicated days between treatment groups was determined. The ones with FDR adjusted p-values≦0.05 were declared to be statistically significant.

For studies 6 and 7, to determine significance of tumor growth inhibition, tumor volumes at 12 days after first dose were compared between the different treatment groups. Treatments were compared by standard ANOVA methods followed by FDR adjustment for multiplicity. Response is square root of volume, for homoscedasticity (equal variance) reasons. Kaplan-Meier (KM) method was carried out to estimate the survival probability of different treatment groups at a given time. For these survival analyses, “Death” means crossing the tumor volume cutoff (2000 mm3). “Survival” means proportion of mice not “Dead”, and “Survival time” means days until “Death”. If a mouse crossed the volume cutoff between two measurement days, then the day of “death” was estimated by linear interpolation. If a mouse crossed the volume cutoff more than once, the first crossing was used. Treatments were compared by the standard log-rank test for two treatments. The log-rank p-values were adjusted for multiplicity using the FDR (false discovery rate) method. Significance was defined as FDR<=0.05. All calculations and graphs were done using R software, version 3.2.3.

### Results

Six studies (Studies 1 through 4 and Studies 6 through 7) were conducted to assess tumor size and survival time in mice treated with CRX601 and rat anti-mouse OX40 Receptor antibody clone OX86, both alone and in combination with each other. One additional study (Study 5 below) was conducted to assess cytokine release and T cell activation in mice treated with CRX601 and rat anti-mouse OX40 Receptor antibody clone OX86, both alone and in combination with each other.

### Study 1

In order to determine CRX-601 monotherapy activity with intratumoral dosing, mice were inoculated with 5×10⁴ CT-26 cells and randomized into groups of 10 listed below when tumor size reached approximately 100 mm³ as described in Materials and Methods.

Group 1: Vehicle dosed intratumoral twice per week for 6 doses total
Group 2: CRX-601 0.1 ug/mouse dosed intratumoral twice per week for 6 doses total
Group 3: CRX-601 1 ug/mouse dosed intratumoral twice per week for 6 doses total
Group 4: CRX-601 10 ug/mouse dosed intratumoral twice per week for 6 doses total
Group 5: CRX-601 50 ug/mouse single dose

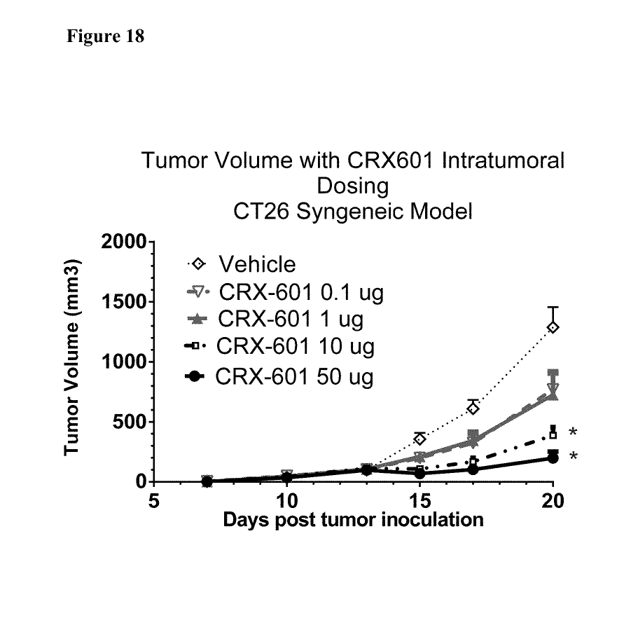

With intratumoral dosing, dose-dependent anti-tumor activity (as measured by tumor growth inhibition over time) was observed for the TLR4 agonist CRX-601 in the CT-26 syngeneic mouse tumor model. The 10 μg and 50 μg dosed mice showed statistically significant (*p-values≦0.05) tumor growth inhibition 11 days after the initial dose compared to vehicle. Results are shown in FIG. 18.

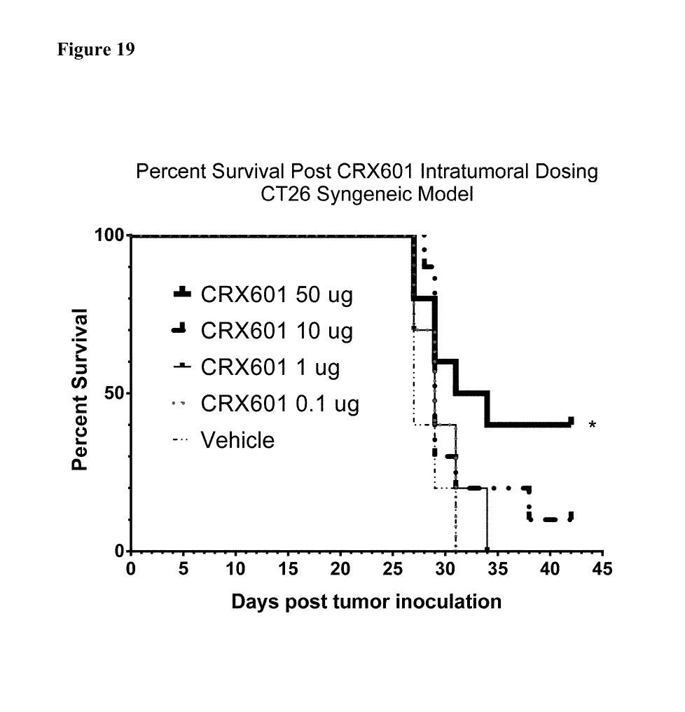

Mice treated with the TLR4 agonist CRX-601 in this study also showed a statistically significant increase in survival time. The 50 μg dosed mice showed a statistically significant (*p-values≦0.05) increase in survival compared to vehicle by day 42 post CT26 tumor cell inoculation when the study was ended. On this day, only mice from the 50 ug and 10 ug CRX-601 groups remained on study. Three of the four mice in the 50 μg group were tumor-free, with the fourth mouse showing a tumor volume of 854.19 mm³. The single mouse remaining in the 10 μg group was tumor-free. (see FIG. 19).

### Study 2

In order to determine CRX-601 monotherapy activity with intravenous dosing, mice were inoculated with 5×10⁴ CT-26 cells and randomized into groups of 10 below when tumor size reached approximately 100 mm³ as described in Materials and Methods.

Group 1: Vehicle dosed intravenous twice per week for 6 doses total
Group 2: CRX-601 1 ug/mouse dosed intravenous twice per week for 6 doses total
Group 3: CRX-601 10 ug/mouse dosed intravenous twice per week for 6 doses total
Group 4: CRX-601 100 ug/mouse single dose

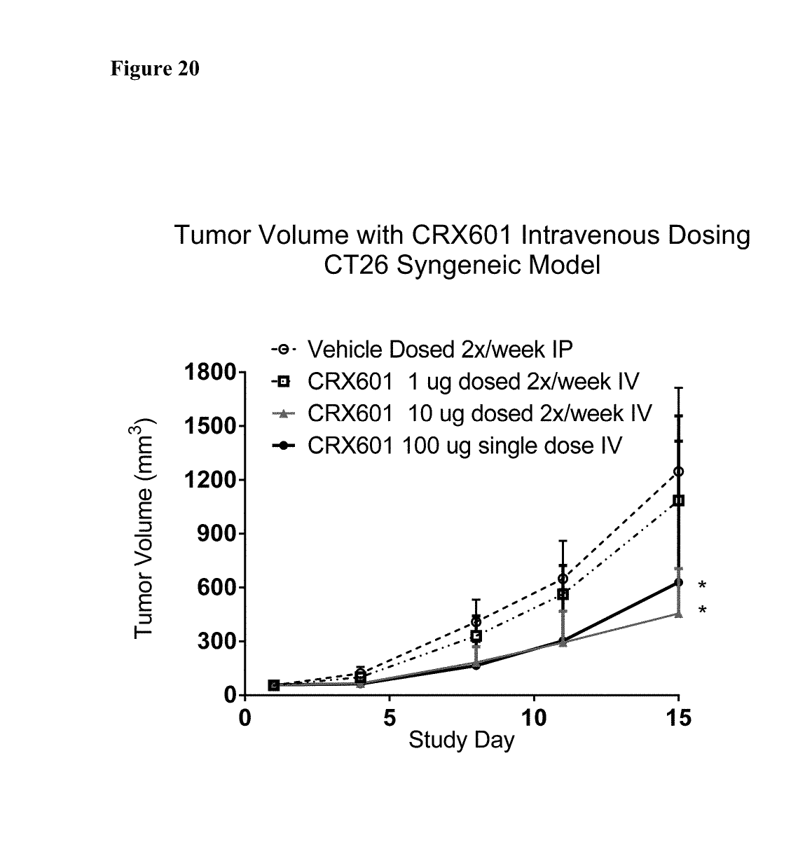

With intravenous dosing, dose-dependent anti-tumor activity (as measured by tumor growth inhibition over time) was observed for the TLR4 agonist CRX-601 in this CT-26 syngeneic mouse tumor model. The 10 μg and 100 μg dosed mice showed statistically significant (*p-values≦0.05) tumor growth inhibition 15 days after the initial dose compared to vehicle (see FIG. 20).

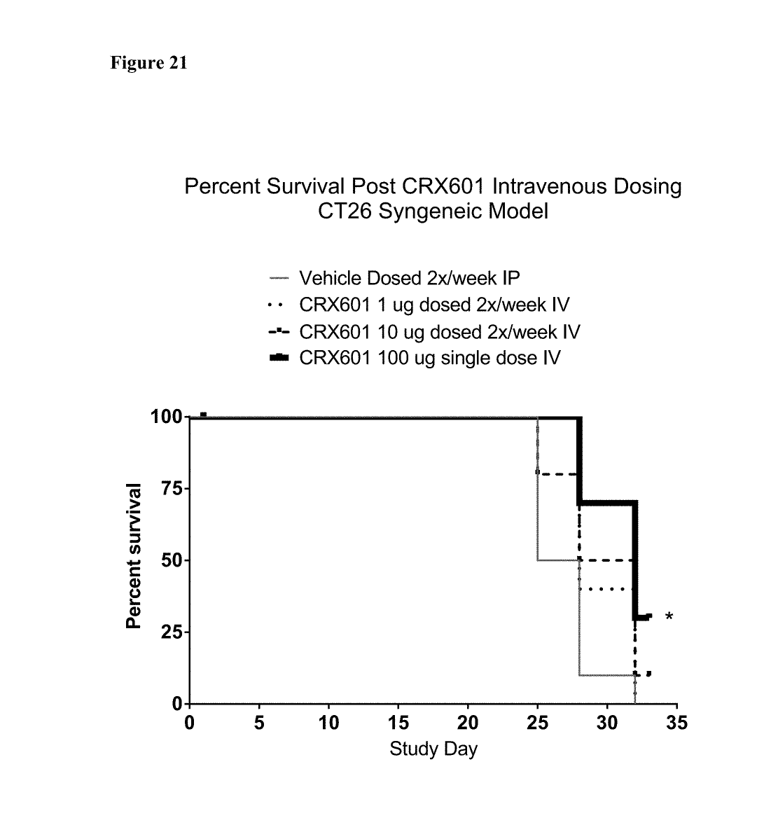

Mice treated with the TLR4 agonist CRX-601 in this CT-26 syngeneic mouse tumor model also showed statistically significant increase in survival compared with vehicle. The 100 μg dosed mice showed a statistically significant increase (*p-values≦0.05) in survival compared to vehicle when the study was ended on day 32 post CT-26 tumor cell inoculation. One of the three mice remaining in this group was tumor-free, while the other mice showed tumor volumes of 1500.49 and 962.61 mm³. The single mouse remaining in the 10 μg dose group had a tumor volume of 188.0 mm³. (See FIG. 21)

### Study 3

In order to determine CRX-601 activity alone and in combination with anti-OX40, mice were inoculated with 5×10⁴ CT-26 cells and randomized into groups of 10 below when tumor size reached approximately 100 mm³ as described in Materials and Methods.

Group 1: Vehicle dosed intravenous once per week for 3 doses total
Group 2: Rat IgG1 10 ug/mouse dosed intraperontoneal twice per week for 6 doses total
Group 3: OX86 25 ug/mouse dosed twice per week for 6 doses total
Group 4: CRX-601 10 ug/mouse dosed intravenous once per week for 3 doses total
Group 5: CRX-601 25 ug/mouse dosed intravenous once per week for 3 doses total
Group 6: CRX-601 10 ug/mouse dosed intravenous once per week for 3 doses total+OX86 25 ug/mouse dosed intraperontoneal twice per week for 6 doses total
Group 7: CRX-601 25 ug/mouse dosed intravenous once per week for 3 doses total+OX86 25 ug/mouse dosed intraperontoneal twice per week for 6 doses total

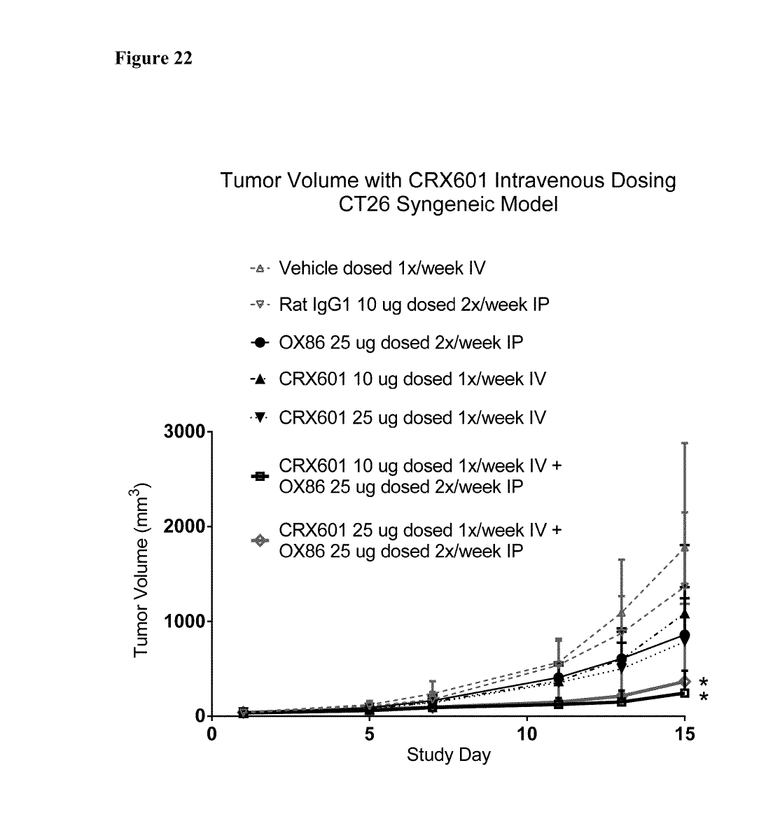

Anti-tumor activity was assessed (as measured by tumor growth inhibition over time) for 25 μg/mouse of a rat anti-mouse OX40 receptor antibody (clone OX-86), dosed intravenous twice per week for 6 doses total, 10 μg or 25 μg/mouse of TLR4 agonist CRX-601 dosed intravenous 1×/week for 3 doses total, and the combination of both in this CT-26 syngeneic mouse model. The sub-optimal monotherapy CRX-601 doses of 10 ug/mouse or 25 ug/mouse dosed once per week did not show statistically significant tumor growth inhibition when dosed alone compared to vehicle, nor did the OX86 25 ug/mouse dose compared to Rat IgG1. However, CRX601 dosed intravenous once per week at 10 μg or 25 μg/mouse for 3 doses total in combination with 25 μg/mouse OX86 dosed twice per week for 6 doses total showed statistically significant (*p-values≦0.05) tumor growth inhibition 15 days after the initial dose compared to vehicle and Rat IgG1 controls, and compared to CRX601 and OX86 monotherapies (see FIG. 22).

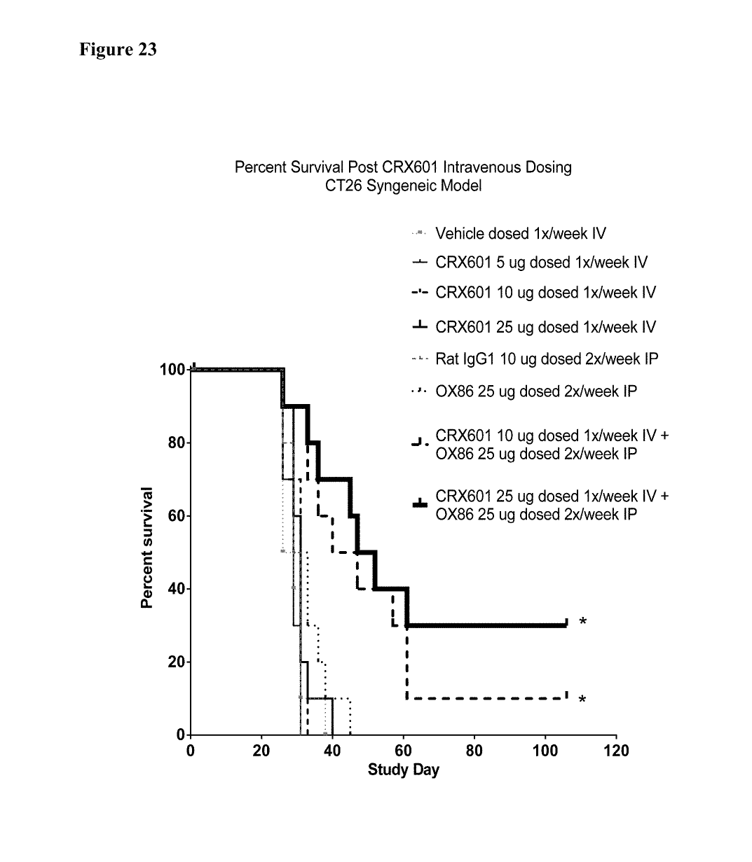

In this CT-26 syngeneic mouse model study, survival advantage was also determined for mice treated with 25 ug/mouse of a rat anti-mouse OX40 receptor antibody (clone OX-86), dosed intravenous twice per week for 6 doses total, 10 μg or 25 μg of TLR4 agonist CRX-601 dosed intravenous 1×/week for 3 doses total, and the combination of both. On day 106 post CT-26 tumor cell inoculation when the study was ended, CRX-601 10 μg and 25 μg/mouse dosed intravenous 1×/week for 3 doses total in combination with 25 μg/mouse OX86 dosed 2×/week for 6 doses total showed a statistically significant (*p-values≦0.05) increase in survival compared to both vehicle and Rat IgG1 controls, and compared to OX86 and CRX-601 monotherapies. The three remaining mice in the CRX-601 25 μg/mouse+OX86 group were tumor-free, and the one mouse in the CRX-601 10 μg/mouse+OX86 group was tumor-free. (see FIG. 23).

### Study 4

Study 3 was repeated with 25 ug/mouse of CRX-601 alone and in combination with anti-OX40. Mice were inoculated with 5×10⁴ CT-26 cells and randomized into groups of 10 below when tumor size reached approximately 100 mm³ as described in Materials and Methods.

Group 1: Vehicle dosed intravenous once per week for 3 doses total+Rat IgG1 25 ug/mouse dosed intraperontoneal twice per week for 6 doses total
Group 2: CRX-601 25 ug/mouse dosed intravenous once per week for 3 doses total
Group 3: Vehicle dosed intravenous once per week for 3 doses total+OX86 25 ug/mouse dosed intraperontoneal twice per week for 6 doses total
Group 4: CRX-601 25 ug/mouse dosed intravenous once per week for 3 doses total+Rat IgG1 25 ug/mouse dosed intraperontoneal twice per week for 6 doses total
Group 5: CRX-601 25 ug/mouse dosed intravenous once per week for 3 doses total+OX86 25 ug/mouse dosed intraperontoneal twice per week for 6 doses total

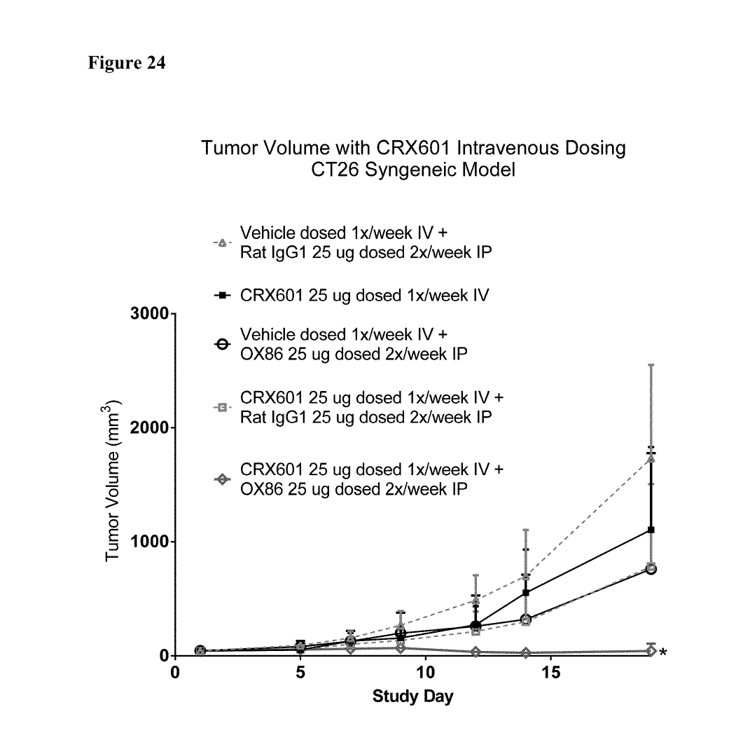

Anti-tumor activity was observed (as measured by tumor volume over time) for 25 μg/mouse of a rat anti-mouse OX40 receptor antibody (clone OX-86), dosed intravenous twice per week for 6 doses total, or 25 μg/mouse of TLR4 agonist CRX-601 dosed intravenous 1×/week for 3 doses total, and the combination of both in a CT-26 syngeneic mouse model. CRX601 dosed intravenous once per week at 25 μg/mouse for 3 doses total in combination with 25 μg/mouse OX86 dosed twice per week for 6 doses total showed statistically significant (*p-values≦0.05) tumor growth inhibition compared to CRX601 and OX86 monotherapies (see FIG. 24).

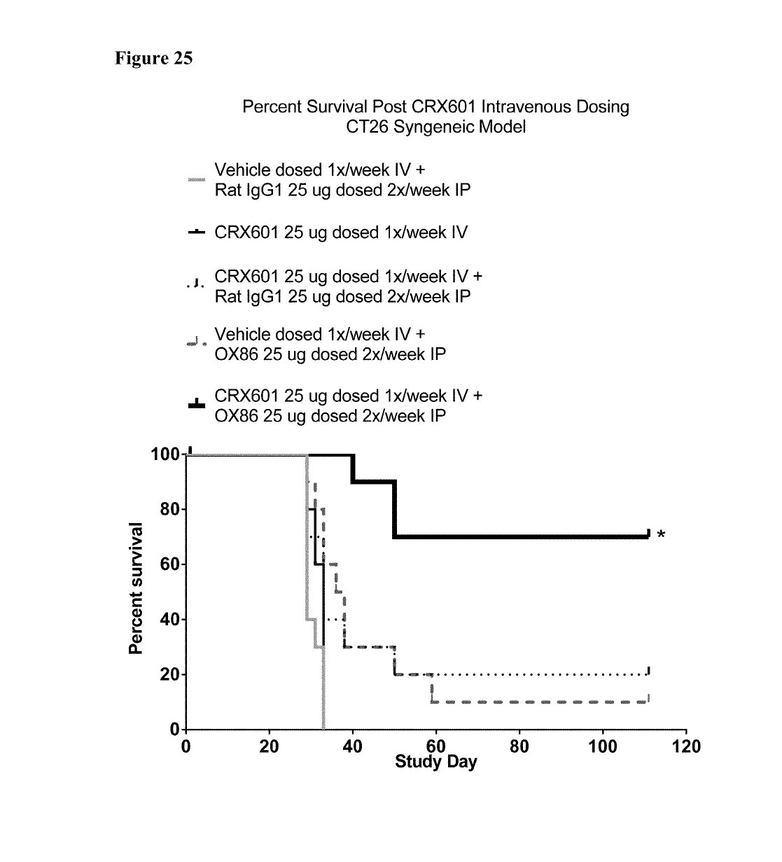

Survival curves were measured for mice treated with 25 μg/mouse of a rat anti-mouse OX40 receptor antibody (clone OX-86), dosed intravenous twice per week for 6 doses total, or 25 μg/mouse of TLR4 agonist CRX-601 dosed intravenous 1×/week for 3 doses total, and the combination of both in a CT-26 syngeneic mouse model. CRX601 25 μg/mouse dosed intravenous 1×/week for 3 doses total in combination with 25 μg/mouse OX86 dosed 2×/week for 6 doses total showed a statistically significant (p-values≦0.05) increase in survival compared to monotherapies. This statistical analysis was conducted on day 64 post tumor cell inoculation when all remaining mice were tumor-free. These mice were monitored until study end on day 111. On this day, seven mice in Group 5 CRX-601 25 ug/mouse+OX86 remained tumor-free, two mice in Group 3 CRX-601 25 ug/mouse+Rat IgG1 remained tumor-free, and one mouse in Group 4 Vehicle+OX86 remained tumor-free. (see FIG. 25).

### Study 5

Results are the mean of five animals per cohort.

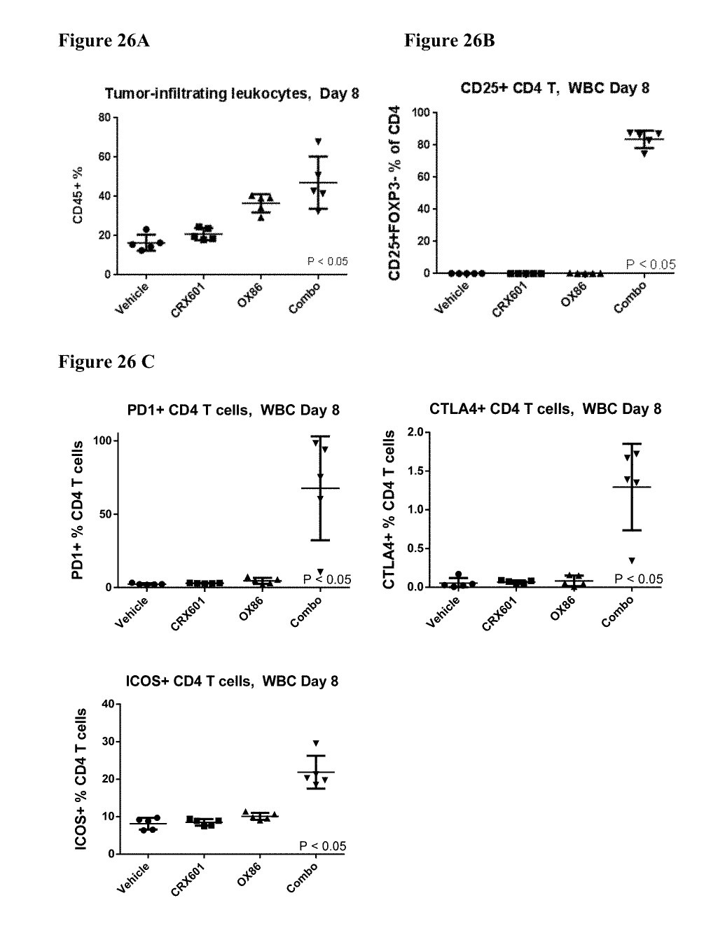

Leukocytes and immune-activation was assessed in mice treated with 10 μg of TLR4 agonist CRX-601, 25 μg of a rat anti-mouse OX40 receptor antibody (clone OX-86), and the combination of both in a CT-26 syngeneic mouse model of colon cancer measured at 8 days post dosing. A significant increase of tumor-infiltrating leukocytes was observed in mice treated with CRX-601 and anti-OX86 in combination. A synergistic increase of expression of T cell activation marker CD25 on circulating CD4 T cells was observed in mice treated with CRX-601 and anti-OX86 in combination. A synergistic increase of T cell activation associated markers CTLA4, PD1 and ICOS on circulating CD4 T cells was observed in mice treated with CRX-601 and anti-OX86 in combination. Results are shown in FIG. 26 A-C.

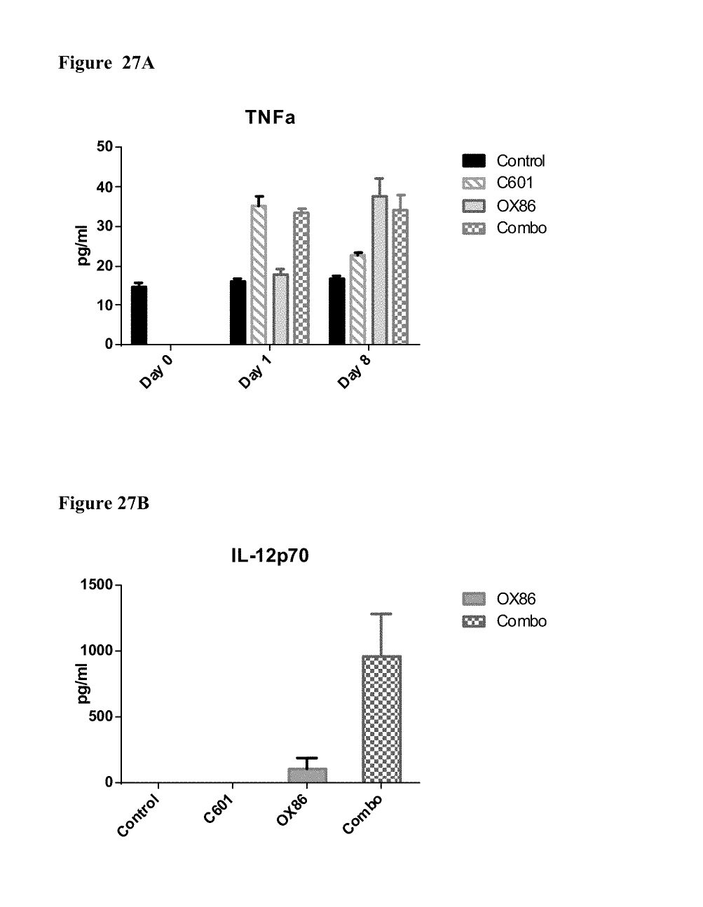

An increase of immune-activating cytokines TNF alpha and IL-12p70 was observed in mice treated with 10 μg of TLR4 agonist CRX-601, a rat anti-mOX40R antibody (OX-86), and the combination of both in a CT-26 syngeneic mouse model of colon cancer measured at 1 and 8 days post dosing. IL-12p70 was only detectable at 8 days post dosing as shown in FIG. 27B. Results are shown in FIGS. 27 A-B.

### Study 6

To compare CRX-601 activity alone and in combination with anti-OX40 when CRX-601 was dosed either (IV) or intratumoral (IT) in a 0.5% glycerol/4% dextrose vehicle, mice were inoculated with 5×10⁴ CT-26 cells and randomized into groups of 10 below when tumor size reached approximately 100 mm³ as described in Materials and Methods.

Group 1: Vehicle dosed intravenous once per week for 3 doses total+Rat IgG1 25 ug/mouse dosed intraperontoneal twice per week for 6 doses total
Group 2: CRX-601 25 ug/mouse dosed intravenous once per week for 3 doses total+Rat IgG1 25 ug/mouse dosed intraperontoneal twice per week for 6 doses total
Group 3: OX86 25 ug/mouse dosed intraperontoneal twice per week for 6 doses total
Group 4: CRX-601 25 ug/mouse dosed intravenous once per week for 3 doses total+OX86 25 ug/mouse dosed intraperontoneal twice per week for 6 doses total
Group 5: Vehicle dosed intratumoral once per week for 3 doses total+Rat IgG1 25 ug/mouse dosed intraperontoneal twice per week for 6 doses total
Group 6: CRX-601 25 ug/mouse dosed intratumoral once per week for 3 doses total+Rat IgG1 25 ug/mouse dosed intraperontoneal twice per week for 6 doses total
Group 7: CRX-601 25 ug/mouse dosed intratumoral once per week for 3 doses total+OX86 25 ug/mouse dosed intraperontoneal twice per week for 6 doses total

Anti-tumor activity was assessed (as measured by tumor growth inhibition over time) for treatment groups. The sub-optimal monotherapy CRX-601 dose of 25 ug/mouse did not show statistically significant tumor growth inhibition when dosed intravenous (Group 2) or intratumoral (Group 6) compared to corresponding control groups (Group 1 and Group 5 respectively). The monotherapy OX86 25 ug/mouse dose did not show statistically significant tumor growth inhibition compared to control Groups 1 and 5 either. However, the CRX601 25 ug/mouse dose given intravenous in combination with the OX86 25 ug/mouse IP dose (Group 4) showed statistically significant (*p-values≦0.05) tumor growth inhibition 12 days after the initial dose compared to control Group 1 and OX86 monotherapy Group 3. The CRX601 25 ug/mouse dose given intratumoral in combination with the OX86 25 ug/mouse IP dose (Group 7) also showed statistically significant (*p-values≦0.05) tumor growth inhibition 12 days after the initial dose compared to control

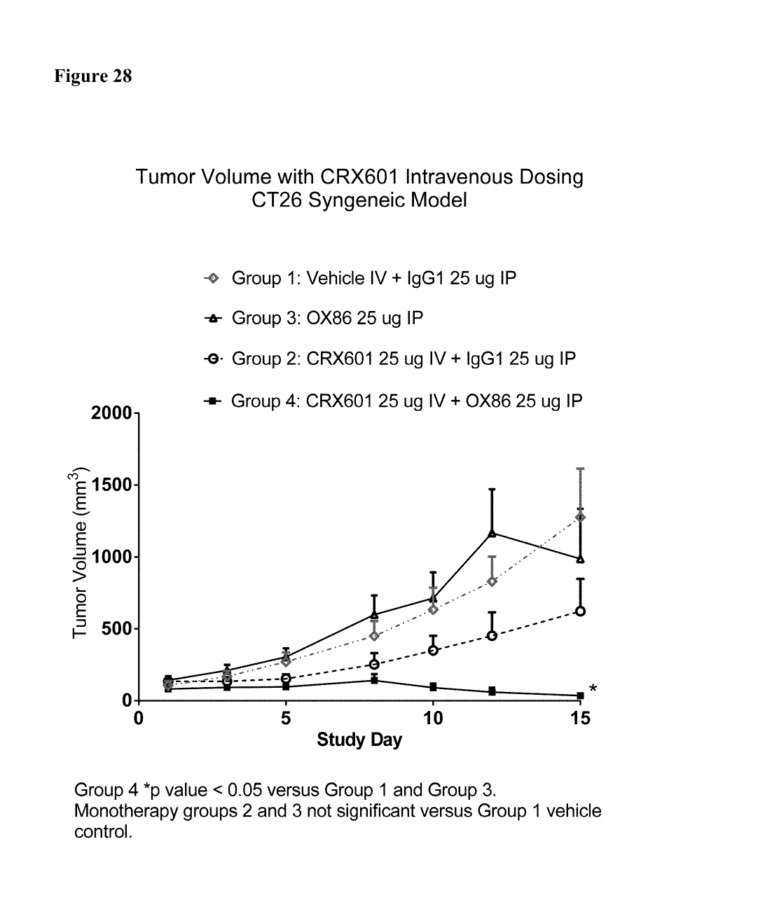

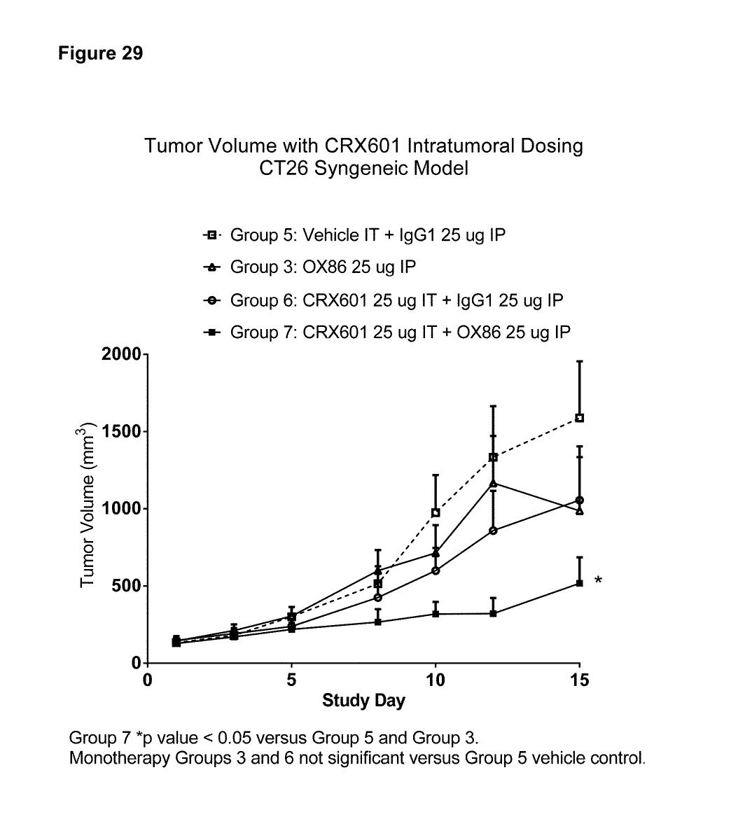

Group 5 and OX86 monotherapy Group 3. The combination of CRX601 25 ug/mouse dosed intravenous (Group 4) or intratumoral (Group 7) with OX86 25 ug/mouse IP was not statistically significant compared to the CRX601 monotherapy Group 2 or Group 6 for tumor growth inhibition in this study (See FIGS. 28 and 29).

In this CT-26 syngeneic mouse model, study survival advantage was also determined. 68 days after the first dose, the combination of CRX601 25 ug/mouse dosed intravenous (Group 4) or intratumoral (Group 7) with OX86 25 ug/mouse IP showed a statistically significant (*p-values≦0.05) increase in survival compared to its control Group 1 or

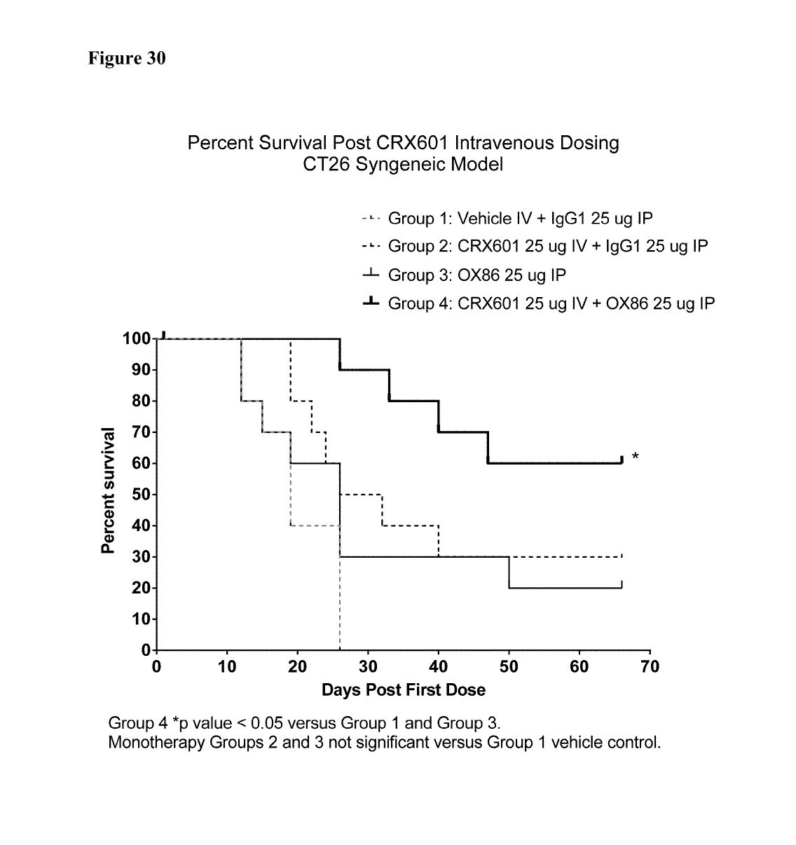

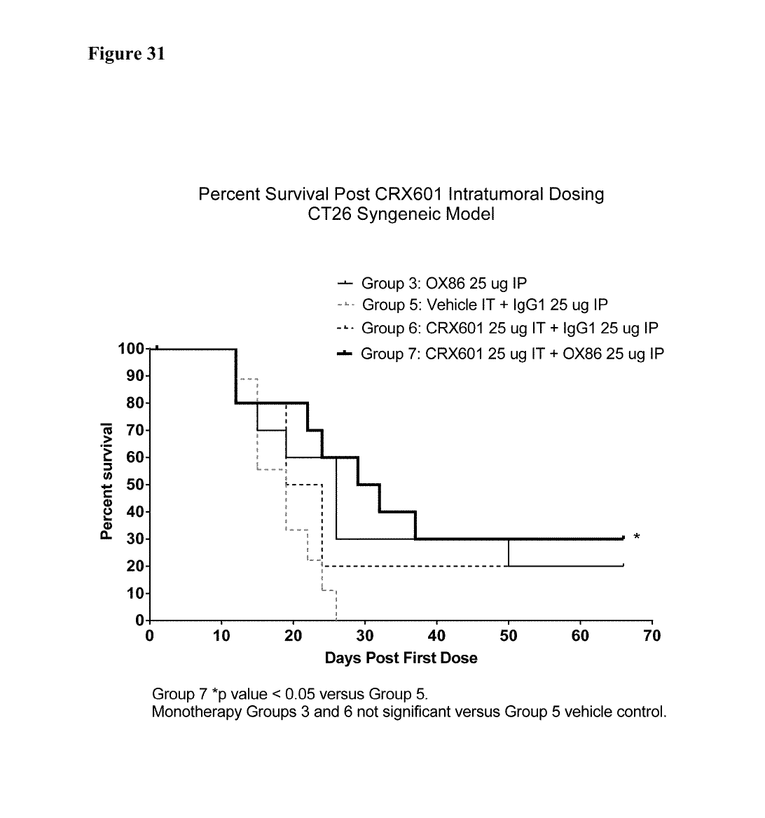

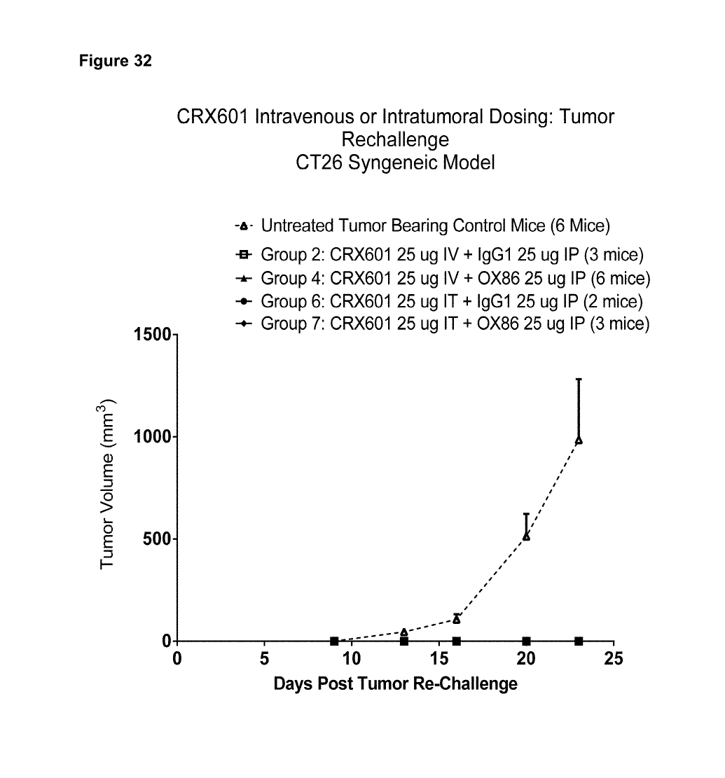

Group 5 respectively. The intravenous dose of CRX-601 in combination with OX86 IP (Group 4) resulted in 6 out of 10 mice tumor-free, and the intratumoral dose of CRX-601 in combination with OX86 IP (Group 7) resulted in 3 out of 10 mice tumor-free. The monotherapy groups did not show a statistically significant increase in survival compared to control groups (see FIGS. 30 and 31). Naïve control mice and fully regressed tumor-free mice on day 68 were re-challenged with CT26 tumor cells. CT26 tumors grew as expected in naïve control mice, but were rejected with no tumor growth in the treatment group mice. This indicates a persistent anti-tumor memory immunity due to CRX-601 or CRX-601 in combination with OX86 treatment (see FIG. 32). The two mice in the OX86 monotherapy Group 3 on day 68 had tumor volumes of 27.86 and 1576.27 mm3, and were not re-challenged.

### Study 7

To compare CRX-601 activity alone and in combination with anti-OX40 when CRX-601 was dosed either intravenous (IV) using a 0.5% Glycerol/4% dextrose vehicle, or intratumoral (IT) using a DOPC/CHOL liposomal formulation, mice were inoculated with 5×10⁴ CT-26 cells and randomized into groups of 10 below when tumor size reached approximately 100 mm³ as described in Materials and Methods

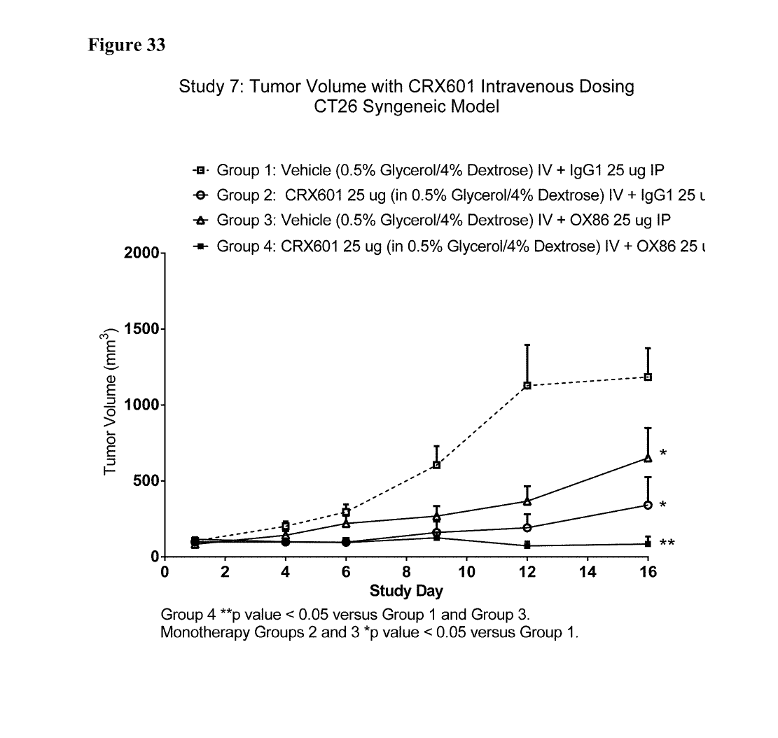

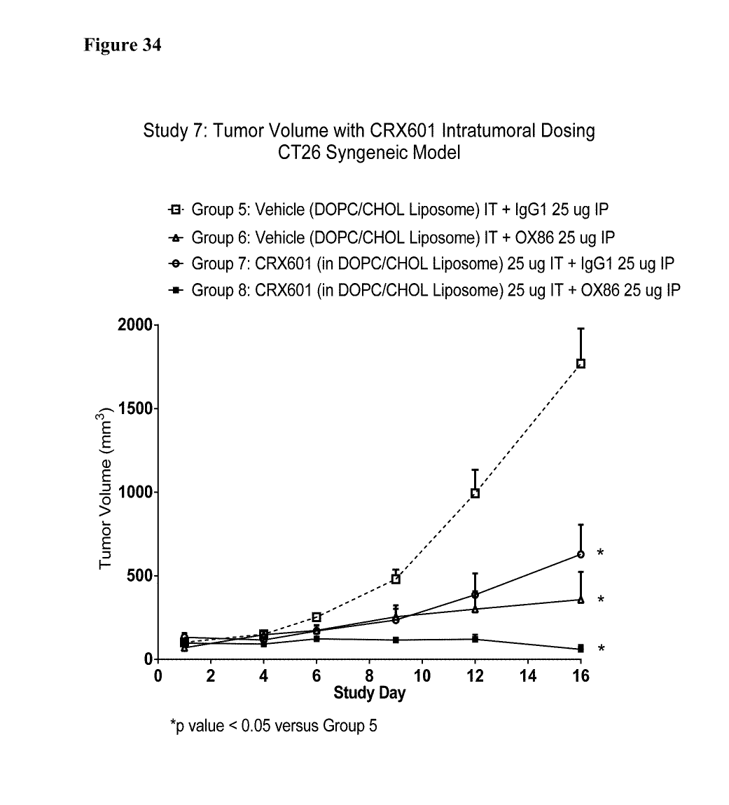

Group 1: Vehicle (0.5% Glycerol/4% dextrose) dosed intravenous once per week for 3 doses total+Rat IgG1 25 ug/mouse dosed intraperontoneal twice per week for 6 doses total
Group 2: CRX-601 25 ug/mouse (in 0.5% Glycerol/4% dextrose) dosed intravenous once per week for 3 doses total+Rat IgG1 25 ug/mouse dosed intraperontoneal twice per week for 6 doses total
Group 3: Vehicle (0.5% Glycerol/4% dextrose) dosed intravenous once per week for 3 doses total+OX86 25 ug/mouse dosed intraperontoneal twice per week for 6 doses total
Group 4: CRX-601 25 ug/mouse (in 0.5% Glycerol/4% dextrose) dosed intravenous once per week for 3 doses total+OX86 25 ug/mouse dosed intraperontoneal twice per week for 6 doses total
Group 5: Vehicle (DOPC/CHOL Liposome) dosed intratumoral once per week for 3 doses total+Rat IgG1 25 ug/mouse dosed intraperontoneal twice per week for 6 doses total
Group 6: Vehicle (DOPC/CHOL Liposome) dosed intratumoral once per week for 3 doses total+OX86 25 ug/mouse dosed intraperontoneal twice per week for 6 doses total
Group 7: CRX-601 25 ug/mouse (in DOPC/CHOL Liposome) dosed intratumoral once per week for 3 doses total+Rat IgG1 25 ug/mouse dosed intraperontoneal twice per week for 6 doses total
Group 8: CRX-601 25 ug/mouse (in DOPC/CHOL Liposome) dosed intratumoral once per week for 3 doses total+OX86 25 ug/mouse dosed intraperontoneal twice per week for 6 doses total Anti-tumor activity was assessed (as measured by tumor growth inhibition over time) for treatment groups 12 days after the initial dose. The sub-optimal monotherapy CRX-601 dose of 25 ug/mouse showed statistically significant (*p-values≦0.05) tumor growth inhibition when dosed intravenous (Group 2) or intratumoral (Group 7, liposomal formulation) compared to corresponding control groups (Group 1 and Group 5 respectively). The monotherapy OX86 25 ug/mouse IP dose Group 3 and Group 7 also showed statistically significant (*p-values≦0.05) tumor growth inhibition compared to control Groups 1 and 5. The CRX601 25 ug/mouse dose given intravenous in combination with the OX86 25 ug/mouse IP dose (Group 4) showed statistically significant (*p-values≦0.05) tumor growth inhibition compared to control Group 1 and OX86 monotherapy Group 3. The CRX601 25 ug/mouse dose given intratumoral with the DOPC/CHOL liposomal formulation in combination with the OX86 25 ug/mouse IP dose (Group 8) also showed statistically significant (*p-values≦0.05) tumor growth inhibition compared to control
Group 5. The combination of CRX601 25 ug/mouse dosed intravenous (Group 4) or intratumoral (Group 8) with OX86 25 ug/mouse IP was not statistically significant compared to the CRX601 monotherapy Group 2 or Group 7 for tumor growth inhibition in this study on day 12 (See FIGS. 33 and 34).

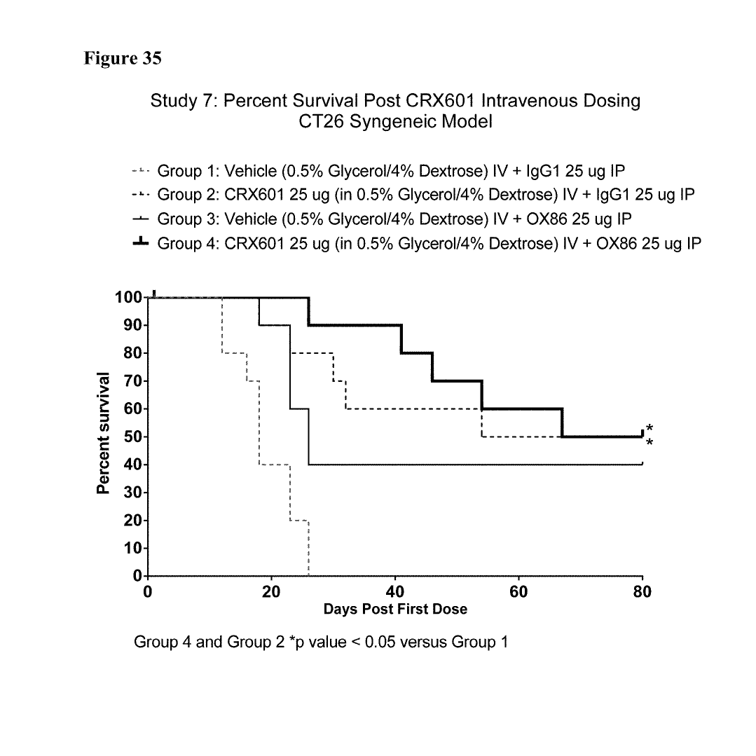

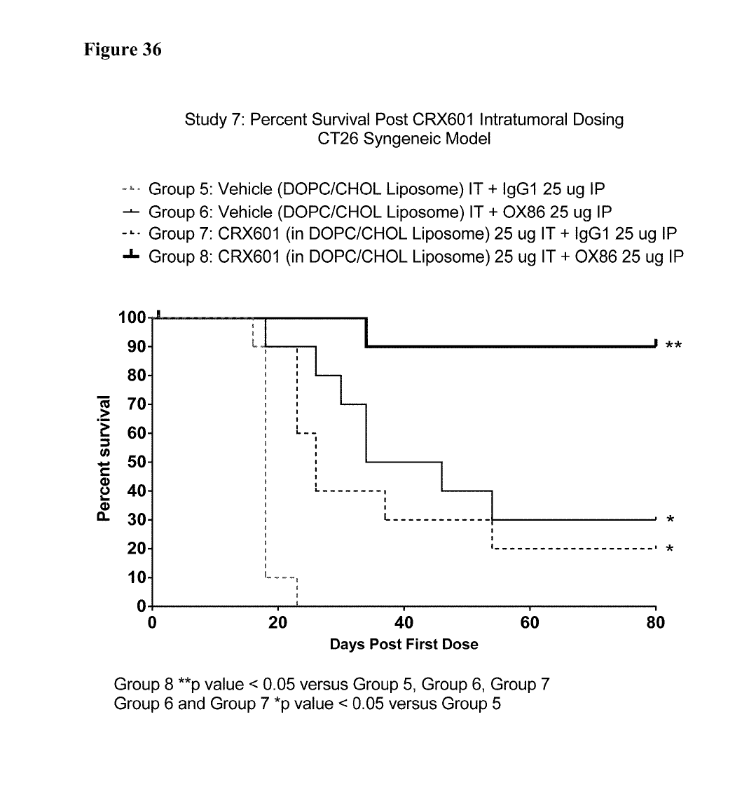

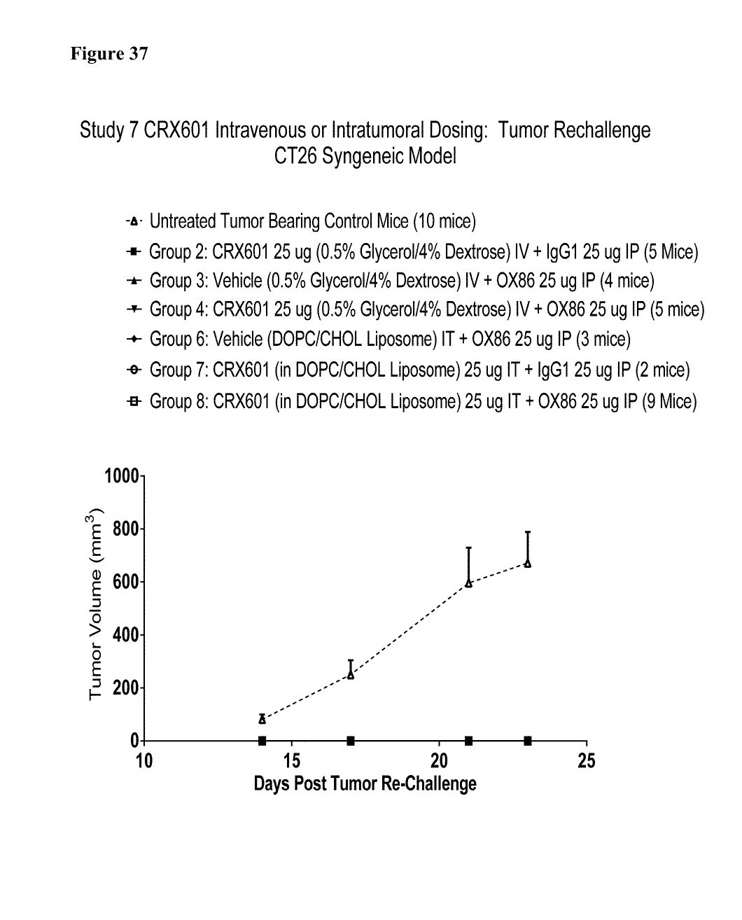

In this CT-26 syngeneic mouse model study, survival advantage was also determined 80 days after the first dose. CRX601 dosed as a monotherapy IV (Group 2), or dosed IV in combination with OX86 IP (Group 4) showed a statistically significant (*p-values≦0.05) increase in survival compared to control Group 1. Groups 2 and 4 had 5 out of 10 mice each showing full tumor regressions (see FIG. 35). Both CRX601 dosed as a monotherapy intratumoral with the DOPC/CHOL liposome formulation (Group 7), and the OX86 monotherapy with the liposomal intratumoral control (Group 6) showed a statistically significant (*p-values≦0.05) increase in survival compared to control Group 5. The intratumoral CRX601 DOPC/CHOL liposomal formulation dose in combination with OX86 IP (Group 8) showed a statistically significant (*p-values≦0.05) increase in survival compared to control Group 5, as well as compared to the CRX601 intratumoral (Group 7) and OX86 (Group 6) monotherapy control groups. 9 out of 10 mice were fully regressed and tumor-free in the intratumoral CRX601 DOPC/CHOL liposomal dose in combination with OX86 IP, compared to 3 and 2 mice in the intratumoral monotherapy control Groups 6 and 7. Thus, synergy was observed with the intratumoral CRX601 liposomal formulation dose in combination with OX86 compared to the intratumoral control monotherapy Groups 6 and 7 (see FIG. 36). Naïve control mice and fully regressed tumor-free mice on day 80 were re-challenged with CT26 tumor cells. CT26 tumors grew as expected in naïve control mice, but were rejected with no tumor growth in the treatment group mice. This result indicates a persistent anti-tumor memory is due to CRX-601 or CRX-601 in combination with OX86 treatment (see FIG. 37). This lack of tumor growth indicates a persistent anti-tumor memory due to CRX-601 or CRX-601 in combination with OX86 treatment (see FIG. 37).

Study 8 An abscopal effect is described as distant tumor regression after a local tumor treatment. In order to asses abscopal effects, mice were inoculated with 5×10⁴ CT-26 cells on the left flank, and 5×10⁴ CT-26 cells on the right flank as described in Materials and Methods for single tumor inoculation. Thus, in this study, each mouse possessed two tumors, one on the right flank, and one on the left flank. Mice were randomized into groups of 10 as shown below when tumor size reached approximately 100 mm³ for the right flank, and left flank tumor size was similar. To determine abscopal effect of CRX-601 activity alone and in combination with anti-OX40, CRX-601 was dosed intratumoral (IT) in the left flank tumor only using a DOPC/CHOL liposomal formulation or a 0.5% glycerol/4% dextrose formulation. Tumor size was monitored for both the right and left flank tumors. In addition, CRX-601 was dosed intravenous (IV) using a 0.5% glycerol/4% dextrose vehicle, alone and in combination with anti-OX40 as a control for systemic activity (Group 7).

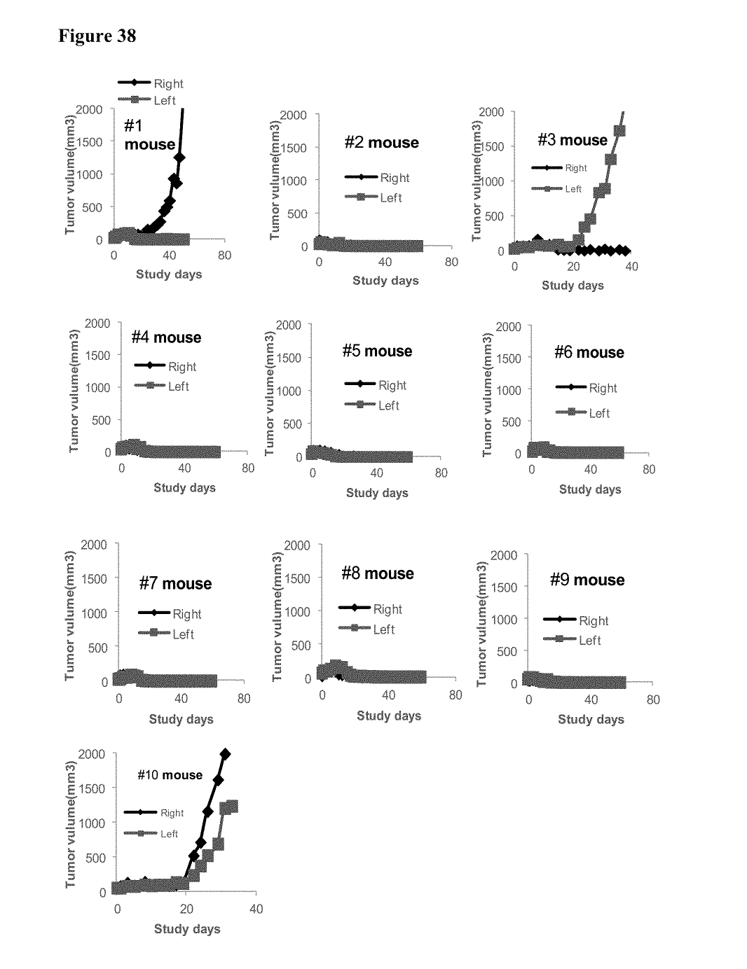

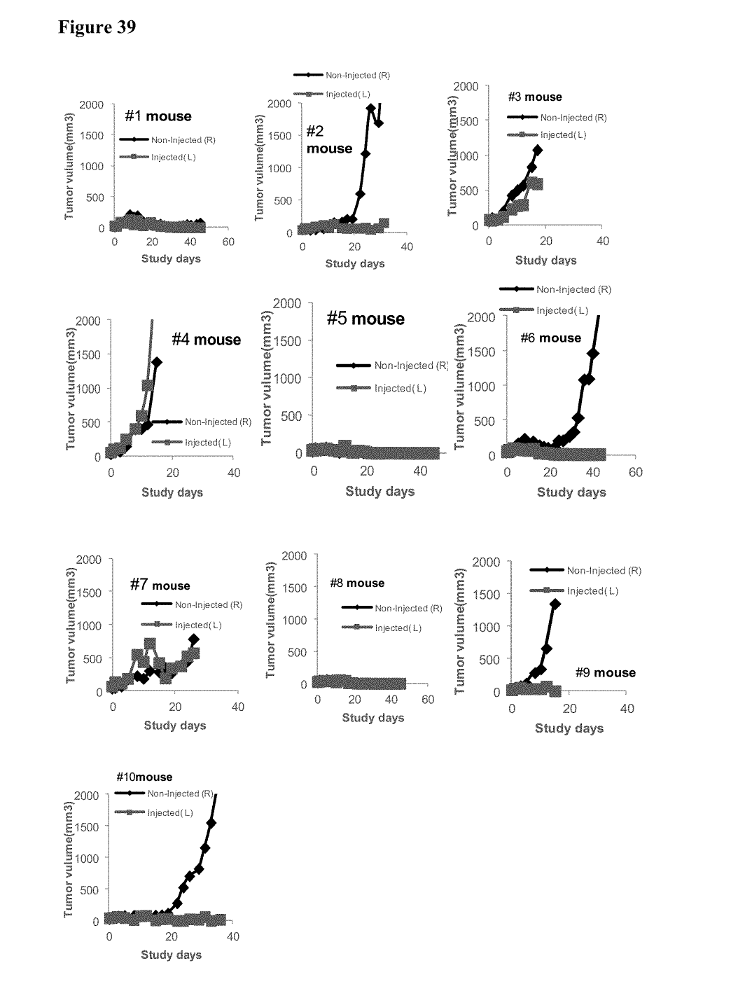

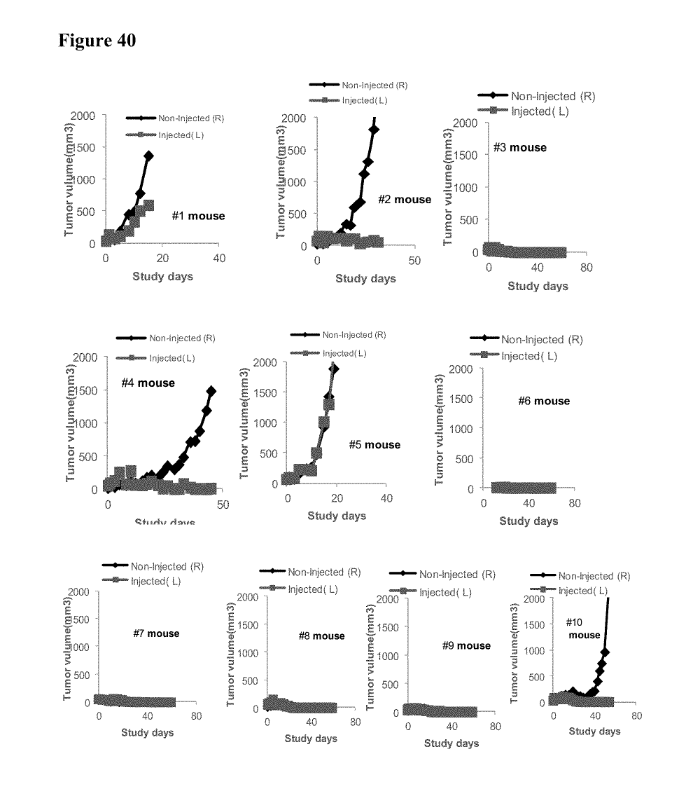

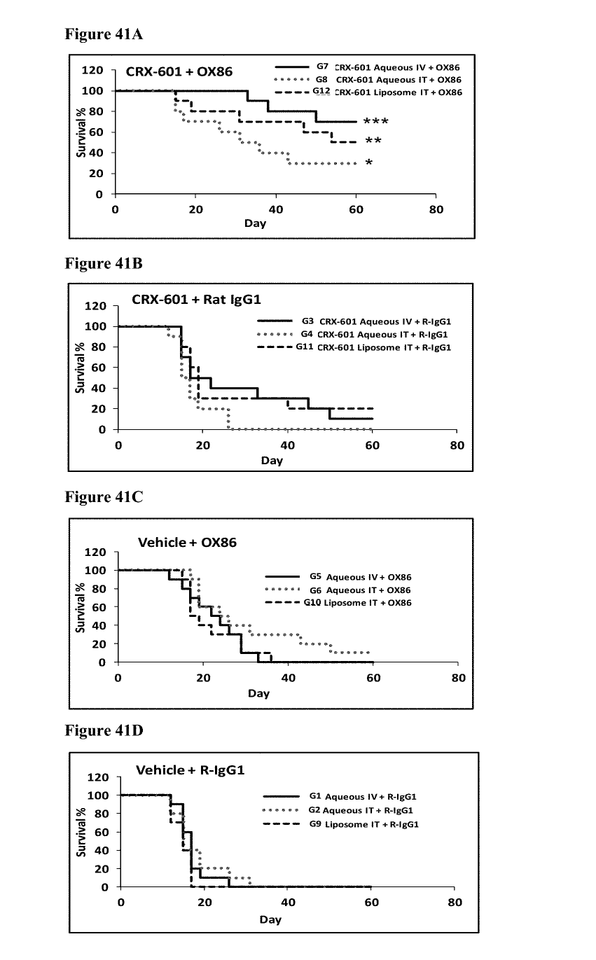

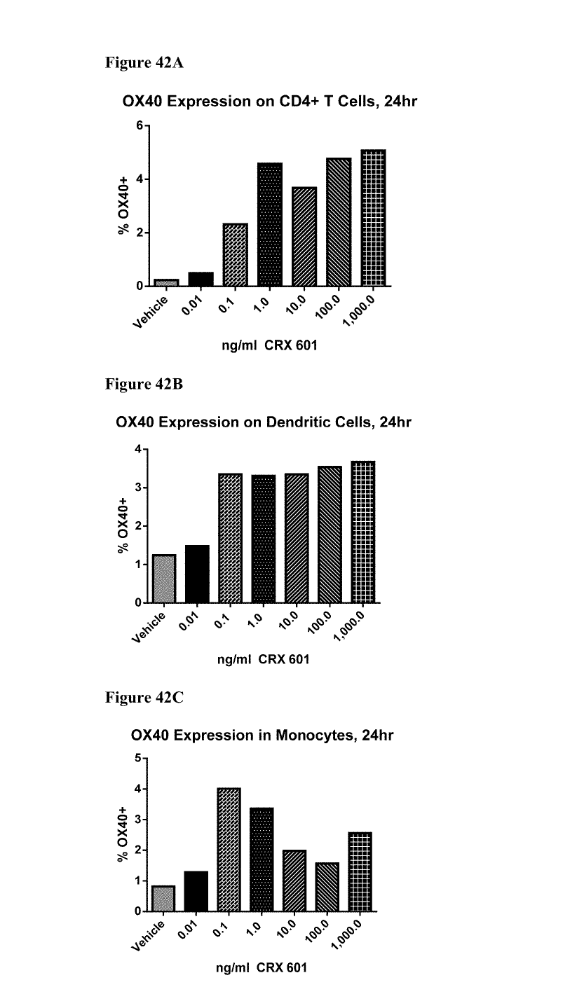

Group 1: Vehicle (0.5% glycerol/4% dextrose) dosed intravenous once per week for 3 doses total+Rat IgG1 25 ug/mouse dosed intraperontoneal twice per week for 6 doses total
Group 2: Vehicle (0.5% glycerol/4% dextrose) dosed intratumoral once per week for 3 doses total+Rat IgG1 25 ug/mouse dosed intraperontoneal twice per week for 6 doses total
Group 3: CRX-601 25 ug/mouse (in 0.5% glycerol/4% dextrose) dosed intravenous once per week for 3 doses total+Rat IgG1 25 ug/mouse dosed intraperontoneal twice per week for 6 doses total
Group 4: CRX-601 25 ug/mouse (in 0.5% glycerol/4% dextrose) dosed intratumoral once per week for 3 doses total+Rat IgG1 25 ug/mouse dosed intraperontoneal twice per week for 6 doses total
Group 5: Vehicle (0.5% glycerol/4% dextrose) dosed intravenous once per week for 3 doses total+OX86 25 ug/mouse dosed intraperontoneal twice per week for 6 doses total
Group 6: Vehicle (0.5% glycerol/4% dextrose) dosed intratumoral once per week for 3 doses total+OX86 25 ug/mouse dosed intraperontoneal twice per week for 6 doses total
Group 7: CRX-601 25 ug/mouse (in 0.5% glycerol/4% dextrose) dosed intravenous once per week for 3 doses total+OX86 25 ug/mouse dosed intraperontoneal twice per week for 6 doses total
Group 8: CRX-601 25 ug/mouse (in 0.5% glycerol/4% dextrose) dosed intratumoral once per week for 3 doses total+OX86 25 ug/mouse dosed intraperontoneal twice per week for 6 doses total
Group 9: Vehicle (DOPC/CHOL Liposome) dosed intratumoral once per week for 3 doses total+Rat IgG1 25 ug/mouse dosed intraperontoneal twice per week for 6 doses total
Group 10: Vehicle (DOPC/CHOL Liposome) dosed intratumoral once per week for 3 doses total+OX86 25 ug/mouse dosed intraperontoneal twice per week for 6 doses total
Group 11: CRX-601 25 ug/mouse (in DOPC/CHOL Liposome) dosed intratumoral once per week for 3 doses total+Rat IgG1 25 ug/mouse dosed intraperontoneal twice per week for 6 doses total
Group 12: CRX-601 25 ug/mouse (in DOPC/CHOL Liposome) dosed intratumoral once per week for 3 doses total+OX86 25 ug/mouse dosed intraperontoneal twice per week for 6 doses total Anti-tumor activity was assessed (as measured by tumor growth inhibition over time) for treatment groups. Mice were removed from study if either or both tumors reached 2,000 mm³. By study day 60 post first dose, all mice remaining on study were completely tumor free, and abscopal effect and survival advantage was determined. For the systemic dosing combination Group 7, CRX-601 25 ug/mouse (in 0.5% Glycerol/4% dextrose) dosed intravenous once per week for 3 doses total+OX86 25 ug/mouse dosed intraperontoneal twice per week for 6 doses total, 7 out of 10 mice were tumor free for both right and left flank tumors (FIG. 38). For the combination Group 8, CRX-601 25 ug/mouse (in 0.5% Glycerol/4% dextrose) dosed intratumoral once per week for 3 doses total+OX86 25 ug/mouse dosed intraperontoneal twice per week for 6 doses total, 3 out of 10 mice showed full tumor regression for both tumors, even though only the left flank tumor received intratumoral injection (FIG. 39). For the combination Group 12, CRX-601 25 ug/mouse (in DOPC/CHOL Liposome) dosed intratumoral once per week for 3 doses total+OX86 25 ug/mouse dosed intraperontoneal twice per week for 6 doses total, 5 out of 10 mice showed full tumor regression for both tumors, even though only the left flank tumor received intratumoral injection (FIG. 40). Thus, CRX-601 formulations dosed intratumoral in combination with OX86 dosed intraperontoneal demonstrated an abscopal effect (Groups 8 and 12). The local left flank tumor IT injection resulted in distant right flank tumor regression. There was no statistical difference in survival advantage between the three combination groups 7, 8, and 12. Group 7 demonstrated a statistically significant increase in survival compared to all vehicle and isotype controls, and also compared to all CRX-601 and OX86 monotherapy groups (***p-values≦0.006). The Group 12 combination showed a statistically significant increase in survival compared to Group 10 Liposome Vehicle IT+OX86 (**p-values=0.006), although it was not statistically significant versus the Group 11 CRX-601 25 ug/mouse Liposome formulation+Rat IgG1 (p-values=0.119). The Group 8 combination showed a statistically significant increase in survival compared to Group 4 CRX-601 25 ug/mouse (in 0.5% Glycerol/4% dextrose) IT+Rat IgG1 (*p-values=0.013), although it was not statistically significant versus the Group 6 Vehicle (0.5% Glycerol/4% dextrose) IT+OX86 (p-values=0.5). FIG. 41 shows the survival curves for all groups.

### Example 6: OX40 Expression Induced by CRX601 Treatment with a Range of Concentrations (0.01-1000 ng/ml) on Human CD4+ T Cells (A), Dendritic Cells (B), and Monocytes (C) at 24 Hours in In Vitro Cell Culture

### Experiment Description:

In vitro human peripheral blood mononuclear cell (PBMC) assay was performed to assess the effect of CRX601 on OX40 expression. Freshly isolated human PBMCs were checked for viability and were cultured in AIM-V serum free media at a density of two million cells per well in a 24-well non tissue culture treated plate. PBMCs were stimulated with a dose concentration (0.01 μg/ml-1,000 μg/ml, including a vehicle blank) of CRX-601 for 24 hours. By the end of incubation, cells were collected for flow cytometry assessment of OX40 expression. The quick upregulation of OX40 receptor expression by CRX601 on T cells, dendritic cells and monocytes demonstrated that CRX601 upregulates the target of anti-OX40 antibody, which may potentiate the therapeutic activity of anti-OX40 antibody and lead to the synergestic anti-tumor activity of TLR4+OX40 combination in vivo.

We claim:

1. A method of treating cancer in a human patient in need thereof, comprising administering to the patient a combination of an antigen binding protein (ABP) that binds to OX40 and a TLR4 modulator.

2. The method of treatment as claimed in claim 1, wherein the ABP that binds to OX40 and modulates OX40 by mimicking OX40L.

3. The method of treatment as claimed in claim 1, wherein the ABP that binds to OX40 is an OX40 agonist.

4. The method of treatment as claimed in claim 1, wherein the ABP that binds to OX40 is a monoclonal antibody.

5. The method of treatment as claimed in claim 1, wherein the ABP that binds to OX40 is a humanized monoclonal antibody comprising: (a) a heavy chain variable region CDR1 comprising the amino acid sequence set forth in SEQ ID NO: 1; (b) a heavy chain variable region CDR2 comprising the amino acid sequence set forth in SEQ ID NO: 2; (c) a heavy chain variable region CDR3 comprising the amino acid sequence set forth in SEQ ID NO. 3; (d) a light chain variable region CDR1 comprising the amino acid sequence set forth in SEQ ID NO: 7; (e) a light chain variable region CDR2 comprising the amino acid sequence set forth in SEQ ID NO. 8; and (f) a light chain variable region CDR3 comprising the amino acid sequence set forth in SEQ ID NO: 9.

6. The method of treatment as claimed in claim 1, wherein the ABP that binds to OX40 is an antibody comprising a heavy chain variable region comprising an amino acid sequence with at least 90% identity to an amino acid sequence chosen from: SEQ ID NO: 4 and SEQ ID NO: 5, wherein the antibody further comprises a light chain variable region comprising an amino acid sequence with at least 90% identity to an amino acid sequence chosen from: SEQ ID NO: 10 and SEQ ID NO: 11.

7. The method of treatment as claimed in claim 1, wherein the ABP that binds to OX40 is an antibody comprising a heavy chain variable region comprising the amino acid sequence as set forth in SEQ ID NO: 5; and a light chain variable region comprising the amino acid sequence set forth in SEQ ID NO: 11.

8. The method of treatment as claimed claim 1, wherein the TLR4 modulator is a TLR4 agonist.

9. The method of treatment as claimed in claim 1, wherein the TLR4 modulator is an aminoalkyl glucosaminide phosphate (AGP).

10. The method of treatment as claimed in claim 1, wherein the TLR4 modulator is a compound chosen from: Formula I and Formula Ia.

11. The method of treatment as claimed in claim 1, wherein the TLR4 modulator is chosen from: CRX-601; CRX-547; CRX-602; and CRX-527.

12. The method of treatment as claimed in claim 1, wherein the TLR4 modulator is CRX-601, having the formula shown below:

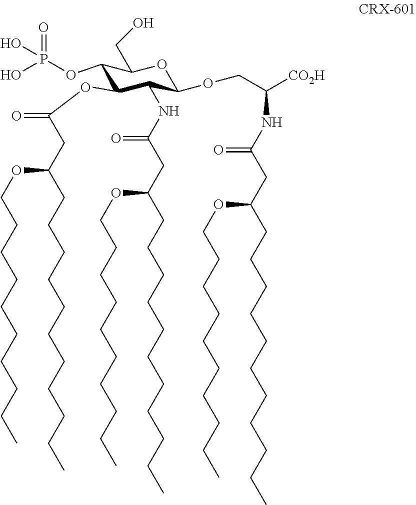

13. A method of treating cancer in a human patient in need thereof, comprising administering to the patient: a first pharmaceutical composition comprising a therapeutically effective amount of an OX40 antibody that binds to human OX40; and a second pharmaceutical composition comprising a therapeutically effective amount of a TLR4 agonist.

14. The method of treatment as claimed in claim 13, wherein the OX40 antibody is a humanized monoclonal antibody comprising: a heavy chain variable region CDR1 comprising an amino acid sequence with at least 90% identity to an amino acid sequence chosen from: SEQ ID NO: 1 and SEQ ID NO: 13; a heavy chain variable region CDR2 comprising an amino acid sequence with at least 90% identity to an amino acid sequence chosen from: SEQ ID NO: 2 and SEQ ID NO: 14; a heavy chain variable region CDR3 comprising an amino acid sequence with at least 90% identity to an amino acid sequence chosen from: SEQ ID NO: 3 and SEQ ID NO: 15; a light chain variable region CDR1 comprising an amino acid sequence with at least 90% identity to an amino acid sequence chosen from: SEQ ID NO: 7 and SEQ ID NO: 19; a light chain variable region CDR2 comprising an amino acid sequence with at least 90% identity to an amino acid sequence chosen from: SEQ ID NO: 8 and SEQ ID NO: 20; and a light chain variable region CDR3 comprising an amino acid sequence with at least 90% identity to an amino acid sequence chosen from: SEQ ID NO: 9 and SEQ ID NO: 21.

15. The method of treatment as claimed in claim 13, wherein the OX40 antibody is a humanized monoclonal antibody comprising: (a) a heavy chain variable region CDR1 comprising the amino acid sequence set forth in SEQ ID NO: 1; (b) a heavy chain variable region CDR2 comprising the amino acid sequence set forth in SEQ ID NO: 2; (c) a heavy chain variable region CDR3 comprising the amino acid sequence set forth in SEQ ID NO. 3; (d) a light chain variable region CDR1 comprising the amino acid sequence set forth in SEQ ID NO: 7; (e) a light chain variable region CDR2 comprising the amino acid sequence set forth in SEQ ID NO. 8; and (f) a light chain variable region CDR3 comprising the amino acid sequence set forth in SEQ ID NO: 9.

16. The method of treatment as claimed in claim 13, wherein the OX40 antibody comprises: a VH region having an amino acid sequence chosen from: an amino acid sequence at least 90% identical to the amino acid sequence as set forth in SEQ ID NO:4; an amino acid sequence at least 90% identical to the amino acid sequence as set forth in SEQ ID NO:5, and VL having an amino acid sequence chosen from: an amino acid sequence at least 90% identical to the amino acid sequence as set forth in SEQ ID NO:10; and an amino acid sequence at least 90% identical to the amino acid sequence as set forth in SEQ ID NO: 11, and wherein the TLR4 agonist is CRX-601, having the formula shown below:

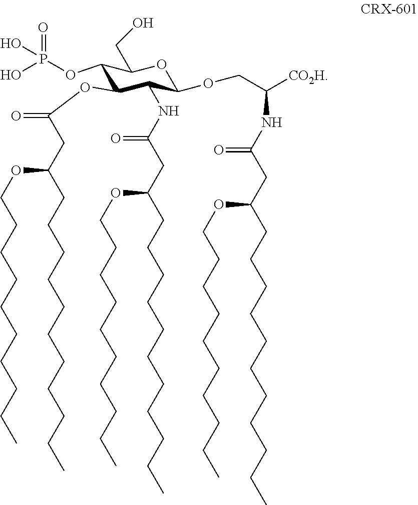

17. The method of treatment as claimed in claim 13, wherein the OX40 antibody that binds to OX40 is an antibody comprising a heavy chain variable region comprising the amino acid sequence as set forth in SEQ ID NO: 5; and a light chain variable region comprising the amino acid sequence set forth in SEQ ID NO: 11.

18. The method of treatment as claimed in claim 13, wherein the OX40 antibody and the TLR4 agonist are administered to the patient via a route chosen from: systemically; intravenously; and intratumorally.

19. The method of treatment as claimed in claim 13, wherein the cancer is chosen from: melanoma; lung cancer; non-small cell lung cancer (NSCLC); kidney cancer; renal cell carcinoma (RCC); breast cancer; metastatic breast cancer; triple-negative breast cancer (TNBC); head and neck cancer; colon cancer; colorectal cancer (CRC); ovarian cancer; pancreatic cancer; liver cancer; hepatocellular carcinoma (HCC); prostate cancer; bladder cancer; gastric cancer; liquid tumors; solid tumors; hematopoetic tumors; leukemia; non-Hodgkins lymphoma (NHL); lymphoma; and chronic lymphocytic leukemia (CLL).

20. The method of treatment as claimed in claim 13, wherein the human has more than one solid tumor, and wherein the TLR4 agonist is administered intratumorally to at least one solid tumor of said human, and wherein the tumor size of at least one solid tumor into which the TLR4 agonist was not administered is reduced.


SEQUENCE LISTING (47 sequences)

SEQ ID NO: 1 - protein, 5 aa, Mus sp.
DYSMH

SEQ ID NO: 2 - protein, 16 aa, Mus sp.
WINTETGEPTYADDFK

SEQ ID NO: 3 - protein, 13 aa, Mus sp.
PYYDYVSYYAMDY

SEQ ID NO: 4 - protein, 122 aa, Mus sp.
QIQLVQSGPELKKPGETVKISCKASGYTFTDYSMHWVKQAPGKGLKWMGWINTETGEPTYADDFKGRFAFSLETSASTAYLQINNLKNEDTATYFCANPYYDYVSYYAMDYWGHGTSVTVSS

SEQ ID NO: 5 - protein, 122 aa, Artificial Sequence
  Synthetic polypeptide
QVQLVQSGSELKKPGASVKVSCKASGYTFTDYSMHWVRQAPGQGLKWMGWINTETGEPTYADDFKGRFVFSLDTSVSTAYLQISSLKAEDTAVYYCANPYYDYVSYYAMDYWGQGTTVTVSS

SEQ ID NO: 6 - DNA, 458 bp, Artificial Sequence
  Synthetic polynucleotide
actagtaccaccatggcttgggtgtggaccttgctattcctgatggcagctgcccaaagtatccaagcacaggttcagttggtgcagtctggatctgagctgaagaagcctggagcctcagtcaaggtttcctgcaaggcttctggttataccttcacagactattcaatgcactgggtgcgacaggctccaggacaaggtttaaagtggatgggctggataaacactgagactggtgagccaacatatgcagatgacttcaagggacggtttgtcttctctttggacacctctgtcagcactgcctatttgcagatcagcagcctcaaagctgaggacacggctgtgtattactgtgctaatccctactatgattacgtctcttactatgctatggactactggggtcagggaaccacggtcaccgtctcctcaggtaagaatggcctctcaagctt

SEQ ID NO: 7 - protein, 11 aa, Mus sp.
KASQDVSTAVA

SEQ ID NO: 8 - protein, 7 aa, Mus sp.
SASYLYT

SEQ ID NO: 9 - protein, 9 aa, Mus sp.
QQHYSTPRT

SEQ ID NO: 10 - protein, 107 aa, Mus sp.
DIVMTQSHKFMSTSVRDRVSITCKASQDVSTAVAWYQQKPGQSPKLLIYSASYLYTGVPDRFTGSGSGTDFTFTISSVQAEDLAVYYCQQHYSTPRTFGGGTKLEIK

SEQ ID NO: 11 - protein, 107 aa, Artificial Sequence
  Synthetic polypeptide
DIQMTQSPSSLSASVGDRVTITCKASQDVSTAVAWYQQKPGKAPKLLIYSASYLYTGVPSRFSGSGSGTDFTFTISSLQPEDIATYYCQQHYSTPRTFGQGTKLEIK

SEQ ID NO: 12 - DNA, 416 bp, Artificial Sequence
  Synthetic polynucleotide
gctagcaccaccatggagtcacagattcaggtctttgtattcgtgtttctctggttgtctggtgttgacggagacattcagatgacccagtctccatcctccctgtccgcatcagtgggagacagggtcaccatcacctgcaaggccagtcaggatgtgagtactgctgtagcctggtatcaacagaaaccaggaaaagcccctaaactactgatttactcggcatcctacctctacactggagtcccttcacgcttcagtggcagtggatctgggacggatttcactttcaccatcagcagtctgcagcctgaagacattgcaacatattactgtcagcaacattatagtactcctcggacgttcggtcagggcaccaagctggaaatcaaacgtaagtagaatccaaagaattc

SEQ ID NO: 13 - protein, 5 aa, Mus sp.
SHDMS

SEQ ID NO: 14 - protein, 17 aa, Mus sp.
AINSDGGSTYYPDTMER

SEQ ID NO: 15 - protein, 11 aa, Mus sp.
HYDDYYAWFAY

SEQ ID NO: 16 - protein, 120 aa, Mus sp.
EVQLVESGGGLVQPGESLKLSCESNEYEFPSHDMSWVRKTPEKRLELVAAINSDGGSTYYPDTMERRFIISRDNTKKTLYLQMSSLRSEDTALYYCARHYDDYYAWFAYWGQGTLVTVSA

SEQ ID NO: 17 - protein, 120 aa, Artificial Sequence
  Synthetic polypeptide
EVQLVESGGGLVQPGGSLRLSCAASEYEFPSHDMSWVRQAPGKGLELVAAINSDGGSTYYPDTMERRFTISRDNAKNSLYLQMNSLRAEDTAVYYCARHYDDYYAWFAYWGQGTMVTVSS

SEQ ID NO: 18 - DNA, 451 bp, Artificial Sequence
  Synthetic polynucleotide
actagtaccaccatggacttcgggctcagcttggttttccttgtccttattttaaaaagtgtacagtgtgaggtgcagctggtggagtctgggggaggcttagtgcagcctggagggtccctgagactctcctgtgcagcctctgaatacgagttcccttcccatgacatgtcttgggtccgccaggctccggggaaggggctggagttggtcgcagccattaatagtgatggtggtagcacctactatccagacaccatggagagacgattcaccatctccagagacaatgccaagaactcactgtacctgcaaatgaacagtctgagggccgaggacacagccgtgtattactgtgcaagacactatgatgattactacgcctggtttgcttactggggccaagggactatggtcactgtctcttcaggtgagtcctaacttcaagctt

SEQ ID NO: 19 - protein, 15 aa, Mus sp.
RASKSVSTSGYSYMH

SEQ ID NO: 20 - protein, 7 aa, Mus sp.
LASNLES

SEQ ID NO: 21 - protein, 9 aa, Mus sp.
QHSRELPLT

SEQ ID NO: 22 - protein, 111 aa, Mus sp.
DIVLTQSPASLAVSLGQRATISCRASKSVSTSGYSYMHWYQQKPGQPPKLLIYLASNLESGVPARFSGSGSGTDFTLNIHPVEEEDAATYYCQHSRELPLTFGAGTKLELK

SEQ ID NO: 23 - protein, 111 aa, Artificial Sequence
  Synthetic polypeptide
EIVLTQSPATLSLSPGERATLSCRASKSVSTSGYSYMHWYQQKPGQAPRLLIYLASNLESGVPARFSGSGSGTDFTLTISSLEPEDFAVYYCQHSRELPLTFGGGTKVEIK

SEQ ID NO: 24 - DNA, 428 bp, Artificial Sequence
  Synthetic polynucleotide
gctagcaccaccatggagacagacacactcctgttatgggtactgctgctctgggttccaggttccactggtgaaattgtgctgacacagtctcctgctaccttatctttgtctccaggggaaagggccaccctctcatgcagggccagcaaaagtgtcagtacatctggctatagttatatgcactggtaccaacagaaaccaggacaggctcccagactcctcatctatcttgcatccaacctagaatctggggtccctgccaggttcagtggcagtgggtctgggacagacttcaccctcaccatcagcagcctagagcctgaggattttgcagtttattactgtcagcacagtagggagcttccgctcacgttcggcggagggaccaaggtcgagatcaaacgtaagtacacttttctgaattc

SEQ ID NO: 25 - protein, 5 aa, Mus sp.
DAWMD

SEQ ID NO: 26 - protein, 19 aa, Mus sp.
EIRSKANNHATYYAESVNG

SEQ ID NO: 27 - protein, 8 aa, Mus sp.
GEVFYFDY

SEQ ID NO: 28 - DNA, 414 bp, Mus sp.
atgtacttgggactgaactatgtattcatagtttttctcttaaatggtgtccagagtgaagtgaagcttgaggagtctggaggaggcttggtgcaacctggaggatccatgaaactctcttgtgctgcctctggattcacttttagtgacgcctggatggactgggtccgccagtctccagagaaggggcttgagtgggttgctgaaattagaagcaaagctaataatcatgcaacatactatgctgagtctgtgaatgggaggttcaccatctcaagagatgattccaaaagtagtgtctacctgcaaatgaacagcttaagagctgaagacactggcatttattactgtacgtggggggaagtgttctactttgactactggggccaaggcaccactctcacagtctcctca

SEQ ID NO: 29 - protein, 138 aa, Mus sp.
MYLGLNYVFIVFLLNGVQSEVKLEESGGGLVQPGGSMKLSCAASGFTFSDAWMDWVRQSPEKGLEWVAEIRSKANNHATYYAESVNGRFTISRDDSKSSVYLQMNSLRAEDTGIYYCTWGEVFYFDYWGQGTTLTVSS

SEQ ID NO: 30 - DNA, 448 bp, Artificial Sequence
  Synthetic polynucleotide
actagtaccaccatgtacttgggactgaactatgtattcatagtttttctcttaaatggtgtccagagtgaagtgaagctggaggagtctggaggaggcttggtgcaacctggaggatccatgaaactctcttgtgctgcctctggattcacttttagtgacgcctggatggactgggtccgccagtctccagagaaggggcttgagtgggttgctgaaattagaagcaaagctaataatcatgcaacatactatgctgagtctgtgaatgggaggttcaccatctcaagagatgattccaaaagtagtgtctacctgcaaatgaacagcttaagagctgaagacactggcatttattactgtacgtggggggaagtgttctactttgactactggggccaaggcaccactctcacagtctcctcaggtgagtccttaaaacaagctt

SEQ ID NO: 31 - protein, 138 aa, Artificial Sequence; identical to SEQ ID NO: 29
  Synthetic polypeptide

SEQ ID NO: 32 - protein, 11 aa, Mus sp.
KSSQDINKYIA

SEQ ID NO: 33 - protein, 7 aa, Mus sp.
YTSTLQP

SEQ ID NO: 34 - protein, 8 aa, Mus sp.
LQYDNLLT

SEQ ID NO: 35 - DNA, 378 bp, Mus sp.
atgagaccgtctattcagttcctggggctcttgttgttctggcttcatggtgctcagtgtgacatccagatgacacagtctccatcctcactgtctgcatctctgggaggcaaagtcaccatcacttgcaagtcaagccaagacattaacaagtatatagcttggtaccaacacaagcctggaaaaggtcctaggctgctcatacattacacatctacattacagccaggcatcccatcaaggttcagtggaagtgggtctgggagagattattccttcagcatcagcaacctggagcctgaagatattgcaacttattattgtctacagtatgataatcttctcacgttcggtgctgggaccaagctggagctgaaa

SEQ ID NO: 36 - protein, 126 aa, Mus sp.
MRPSIQFLGLLLFWLHGAQCDIQMTQSPSSLSASLGGKVTITCKSSQDINKYIAWYQHKPGKGPRLLIHYTSTLQPGIPSRFSGSGSGRDYSFSISNLEPEDIATYYCLQYDNLLTFGAGTKLELK

SEQ ID NO: 37 - DNA, 413 bp, Artificial Sequence
  Synthetic polynucleotide
gctagcaccaccatgagaccgtctattcagttcctggggctcttgttgttctggcttcatggtgctcagtgtgacatccagatgacacagtctccatcctcactgtctgcatctctgggaggcaaagtcaccatcacttgcaagtcaagccaagacattaacaagtatatagcttggtaccaacacaagcctggaaaaggtcctaggctgctcatacattacacatctacattacagccaggcatcccatcaaggttcagtggaagtgggtctgggagagattattccttcagcatcagcaacctggagcctgaagatattgcaacttattattgtctacagtatgataatcttctcacgttcggtgctgggaccaagctggagctgaaacgtaagtacacttttctgaattc

SEQ ID NO: 38 - protein, 126 aa, Artificial Sequence; identical to SEQ ID NO: 36
  Synthetic polypeptide

SEQ ID NO: 39 - DNA, 20 bp, Artificial Sequence
  Synthetic primer
cgctgttttgacctccatag

SEQ ID NO: 40 - DNA, 20 bp, Artificial Sequence
  Synthetic primer
tgaaagatgagctggaggac

SEQ ID NO: 41 - DNA, 20 bp, Artificial Sequence
  Synthetic primer
ctttcttgtccaccttggtg

SEQ ID NO: 42 - DNA, 19 bp, Artificial Sequence
  Synthetic primer
gctgtcctacagtcctcag

SEQ ID NO: 43 - DNA, 18 bp, Artificial Sequence
  Synthetic primer
acgtgccaagcatcctcg

SEQ ID NO: 44 - DNA, 1407 bp, Artificial Sequence
  Synthetic polynucleotide
atgtacttgggactgaactatgtattcatagtttttctcttaaatggtgtccagagtgaagtgaagctggaggagtctggaggaggcttggtgcaacctggaggatccatgaaactctcttgtgctgcctctggattcacttttagtgacgcctggatggactgggtccgccagtctccagagaaggggcttgagtgggttgctgaaattagaagcaaagctaataatcatgcaacatactatgctgagtctgtgaatgggaggttcaccatctcaagagatgattccaaaagtagtgtctacctgcaaatgaacagcttaagagctgaagacactggcatttattactgtacgtggggggaagtgttctactttgactactggggccaaggcaccactctcacagtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 45 - protein, 469 aa, Artificial Sequence [sequence omitted]

SEQ ID NO: 46 - DNA, 702 bp, Artificial Sequence
  Synthetic polynucleotide
atgagaccgtctattcagttcctggggctcttgttgttctggcttcatggtgctcagtgtgacatccagatgacacagtctccatcctcactgtctgcatctctgggaggcaaagtcaccatcacttgcaagtcaagccaagacattaacaagtatatagcttggtaccaacacaagcctggaaaaggtcctaggctgctcatacattacacatctacattacagccaggcatcccatcaaggttcagtggaagtgggtctgggagagattattccttcagcatcagcaacctggagcctgaagatattgcaacttattattgtctacagtatgataatcttctcacgttcggtgctgggaccaagctggagctgaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 47 - protein, 233 aa, Artificial Sequence
  Synthetic polypeptide
MRPSIQFLGLLLFWLHGAQCDIQMTQSPSSLSASLGGKVTITCKSSQDINKYIAWYQHKPGKGPRLLIHYTSTLQPGIPSRFSGSGSGRDYSFSISNLEPEDIATYYCLQYDNLLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC